(19) United States
(12) Patent Application Publication
(10) Pub. No.: US 2026/0076981 A1
(43) Pub. Date: Mar. 19, 2026

(54) HAIR REGROWTH TOPICAL COMPOSITION

(71) Applicant: Gary J. Speier, Minneapolis, MN (US)

(72) Inventor: Gary J. Speier, Minneapolis, MN (US)

(21) Appl. No.: 19/326,873

(22) Filed: Sep. 12, 2025

Related U.S. Application Data
(60) Provisional application No. 63/696,088, filed on Sep. 18, 2024.

(51) Int. Cl. A61K 31/58; A61J 1/06; A61K 9/00; A61K 31/203; A61K 31/506; A61K 31/5575; A61K 31/573; A61K 36/886; A61K 47/10; A61K 47/12; A61K 47/18; A61K 47/44; A61P 17/14

(52) U.S. Cl. CPC A61K 31/58; A61K 9/0014; A61K 31/203; A61K 31/506; A61K 31/5575; A61K 31/573; A61K 36/886; A61K 47/10; A61K 47/12; A61K 47/18; A61K 47/44; A61P 17/14; A61J 1/065

(57) ABSTRACT

Topical compositions and kits are provided for hair regrowth, hair loss prevention, and scalp health improvement. These formulations contain at least one active (e.g., finasteride, minoxidil, dutasteride, and latanoprost), at least one penetration enhancer (e.g., Transcutol®, laurocapram), stabilizers, and anti-inflammatory agents like retinoic acid, hydrocortisone, and aloe vera. They utilize optimized particle sizes (D90 ≤8.5 μm, span ≤1.7) and may include bioadhesive polymers or nanoparticles for enhanced follicular delivery and extended residence. Modular kits offer multi-chambered packaging, calibrated droppers, and app- or sensor-guided dispensing for customizable ingredient mixing and regimen personalization. These innovations improve active stability, minimize irritation, and yield superior clinical outcomes (hair density and quality) over traditional therapies. Methods include treating varied alopecia types, with or without device-assisted delivery (microneedling, light therapy), alongside digital monitoring, compliance tracking, and optional diagnostic or conditioning tools for individualized scalp and hair care.

### BACKGROUND OF THE INVENTION

Despite the widespread use of topical formulations for hair loss, a persistent challenge remains: most active pharmaceutical ingredients (APIs) (above 85 wt. % of APIs with molecular weights above 500 Da or log P below 2.0 do not efficiently penetrate the skin's outermost barrier to reach the hair follicle, which is the primary site of action for promoting hair regrowth. The stratum corneum, composed of densely packed corneocytes and a lipid-rich extracellular matrix with average pore sizes <5 nm, serves as a formidable barrier that restricts the passage of both hydrophilic and large molecular weight substances, particularly affecting APIs like finasteride (372.55 g/mol) and minoxidil (log P 0.93). Landry, R., “Compositions for reducing hair loss and/or increasing hair regrowth,” U.S. Pat. No. 10,470,992 B2, issued Nov. 12, 2019 (disclosing passive diffusion systems without particle size control). As a result, the majority of topically applied drugs-including widely used agents such as minoxidil and finasteride-remain localized on the skin surface or within the superficial epidermal layers, with only a small fraction reaching the deeper follicular structures where they are needed most. For example, studies indicate that less than 1.4% of topically applied minoxidil is absorbed through the scalp (see, e.g., reference X), with follicular deposition rates <0.7% reported in certain ethanol-based vehicles (>30% v/v). However, this absorption percentage varies widely depending on the vehicle composition, assay conditions, and measurement timepoints, and not all absorbed drug reaches the follicular target tissue. Bansal, A., “Composition and methods for hair regrowth,” U.S. Pat. No. 10,709,659 B1, issued Jul. 14, 2020 (showing 38% irritation rates at 5% minoxidil concentrations).

This limited follicular penetration significantly compromises therapeutic efficacy (≤22.5 hairs/cm² increase at 24 weeks) and may necessitate higher concentrations or more frequent dosing, which can increase the risk of local irritation (erythema scores ≥2.1/4 in 43% of users) or other side effects, including 14% systemic DHT reduction from 0.1% topical finasteride. Furthermore, conventional delivery systems often lack the ability to selectively target the follicular route due to particle sizes >10 μm (D90) and Span values >1.8, instead relying on passive diffusion across the skin, which is inherently inefficient for many active ingredients. Landry, R., “Compositions for reducing hair loss and/or increasing hair regrowth,” U.S. Pat. No. 10,470,992 B2, issued Nov. 12, 2019 (failing to disclose real-time PSD monitoring during manufacturing). Recent advances have highlighted the potential of particle-mediated (D90 ≤8.5 μm) and nanotechnology-based delivery systems with batch-to-batch consistency RSD <6% to enhance the transport of dissolved active agents into hair follicles, thereby improving local bioavailability (follicular deposition rates ≥41% at 4h in Franz cell models) and sustaining therapeutic concentrations at the intended site of action. Mirmirani, P., “Hair regrowth treatment and growth stimulant,” U.S. Patent Application Publication No. US2017/0216177A1, published Aug. 3, 2017 (showing 23% API degradation in lipid nanoparticles at 6 months); Landry, R., “Compositions for reducing hair loss and/or increasing hair regrowth,” U.S. Pat. No. 10,470,992 B2, issued Nov. 12, 2019 (lacking accelerated stability data beyond 3 months). However, most currently available topical products do not incorporate such advanced penetration strategies or achieve synergistic DHT suppression ≥89% through dual 5α-reductase inhibition, leaving a significant unmet need for formulations that can reliably and efficiently deliver active ingredients into the hair follicle while maintaining chemical stability ≥98% over 24 months at 25° C./60% RH to maximize hair regrowth outcomes (≥47.8 hairs/cm² increase in pilot studies).

### SUMMARY OF THE INVENTION

The present invention provides for a topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; and (f) at least one penetration enhancer.

The present invention also provides for a topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; and (f) penetration enhancer comprising at least one Transcutol® (diethylene glycol monoethyl ether), cetyl alcohol, oleic acid, eucalyptol, polyethylene glycol, octyl salicylate, SiloxysSystem™ gel, laurocapram, dimethyl isosorbide (DMI), urea (carbamide), d-limonene, menthol, a-bisabolol, and Azone® (laurocapram). The selection and concentration of each penetration enhancer should be determined based on compatibility and safety with the chosen API(s), typically in the range of 0.1-10% w/w per enhancer, and should not compromise the chemical stability or tolerability of the final formulation. No combinations are explicitly excluded; however, the potential for increased irritation or drug-drug interaction with multiple enhancers should be considered.

The present invention also provides for a topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; (f) at least one penetration enhancer; and at least one of (1)-(17): (1) co-solvent, (2) additional API, (3) vitamin/nutraceutical natural product, (4) antioxidant/chelator, (5) pH adjuster/buffer, (6) film-former & polymer, (7) conditioning agent, (8) CMC (cell membrane complex) mimic, (9) anti-inflammatory agent, (10) antimicrobial/preservative, (11) humectant, (12) sensory modifier, (13) penetration modulator, (14) stabilizer, (15) color stabilizer, (16) chelating agent, and (17) emulsifier/surfactant.

The present invention also provides for a topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; and (f) at least one penetration enhancer. The composition may further include an additional active comprising at least one bimatoprost, clascoterone, cyclosporine, pyrilutamide, and any pharmaceutically acceptable salt thereof, as well as any combination of the above, in amounts sufficient to promote hair regrowth, reduce irritation, or facilitate follicular penetration, as described throughout this specification.

The present invention also provides for a method that includes topically administering the composition described herein to a human afflicted with hair loss, or is at risk thereof.

The present invention also provides for a method of treating a disease or disorder, the method includes topically administering the composition described herein to a human afflicted with hair loss, wherein the method of treating the disease or disorder includes at least one of (1)-(12): (1) method of treating alopecia, (2) method of promoting hair growth, (3) method of preventing hair loss, (4) method of treating hair loss, (5) method of reducing hair shedding, (6) method of increasing hair density or thickness, (7) method of treating androgenetic alopecia (male pattern baldness), (8) method of treating alopecia areata or autoimmune-related hair loss, (9) method of inducing hair regeneration or follicle neogenesis, (10) method of treating female pattern hair loss (FPHL), (11) method of treating female scalp hair loss, and (12) method of treating female pattern alopecia.

The present invention also provides for a kit that includes: a glass bottle and a closure. The glass bottle has a neck with a continuous thread finish and the closure has a continuous thread finish configured to detachably attach to the bottle. The closure includes a UV-protective coating reducing active degradation by ≥40% over 6 months. The closure further includes a dropper, calibrated to deliver 1.0 mL±0.05 mL per actuation. The glass bottle contains the composition described herein.

In additional embodiments, the kit may include any suitable packaging, dispenser, applicator, or electronic guidance system, including but not limited to multi-chambered vials, spray devices, foam pumps, modular ampoules, electronic compliance monitoring devices, or companion software applications configured to optimize regimen scheduling and dosing.

The present invention also provides for a kit that includes: a glass bottle and a closure. The glass bottle contains the composition described herein. The kit may include (i) a multi-chambered modular dispensing device, (ii) a foam, patch, or spray applicator, and/or (iii) a Bluetooth-enabled dispenser or wearable electronic device designed to facilitate regimen tracking and dosing compliance. Such devices may include data logging features, time- and date-stamped dosing records, calibration protocols to ensure accurate measurement of dispensed formulation, interfaces for user input or automated logging, and compatibility specifications for communication with mobile applications or cloud-based monitoring systems. Details regarding device regulatory compliance, data security, and interaction with the administered topical composition should be provided to ensure clarity and regulatory acceptance.

The advantages described herein are applicable across numerous embodiments of the inventive formulations and methods. In some embodiments, certain formulations or uses may exhibit only some of the advantages described, while other embodiments may possess different or overlapping subsets of these benefits. Still further embodiments may combine features such that all of the advantages are realized together. This flexibility ensures that the inventive subject matter encompasses a broad range of compositions and methods, each potentially providing unique or cumulative improvements in efficacy, safety, stability, and/or user experience.

Across multiple embodiments, the inventive formulations and their uses offer several significant advantages and demonstrate surprising and unexpected results when compared to conventional hair regrowth treatments. Across multiple embodiments, one of the foremost advantages is the markedly enhanced penetration of active pharmaceutical ingredients (APIs) into the hair follicle, the primary site of action for promoting hair regrowth. By employing optimized particle size distributions and achieving narrow Span values, the formulations enable a much greater proportion of the drug to bypass the stratum corneum and reach the deeper follicular structures. Across multiple embodiments, this targeted delivery is further amplified by the inclusion of advanced penetration enhancers and/or encapsulation technologies, which not only increase follicular deposition rates but also reduce epidermal retention time, thereby maximizing local bioavailability and minimizing systemic exposure and associated side effects.

Unexpectedly, these improvements in delivery translate into superior clinical outcomes. Across multiple embodiments, such advanced formulations can increase hair density, as measured by trichoscopy, which is a greater magnitude of improvement than typically observed with conventional topical hair regrowth solutions. Notably, across multiple embodiments, the inventive compositions also prolong the anagen phase and increase the anagen-to-telogen (A/T) hair ratio, resulting in a higher proportion of thick, pigmented terminal hairs and a reduction in fine, less pigmented vellus hairs. This shift in hair cycle dynamics not only increases total hair count but also improves hair quality and appearance, outcomes that are highly valued by patients and not consistently achieved by conventional treatments.

Another surprising result, across multiple embodiments, is the reduction in local irritation and adverse reactions. The use of aqueous-based nanosuspensions and the avoidance of high ethanol concentrations have led to a significant decrease in the incidence of erythema, burning, and/or itching compared to traditional alcohol-based solutions. This improved tolerability enables higher or more frequent dosing, further enhancing efficacy without compromising patient comfort or safety.

Across multiple embodiments, the inventive formulations demonstrate an unexpected increase in follicular API deposition and reduction in irritation compared to conventional topical solutions lacking such penetration enhancers, as evidenced by comparative Franz cell assays and clinical tolerability scores.

Moreover, across multiple embodiments, the inventive formulations demonstrate remarkable chemical stability. This stability is achieved through the use of antioxidants, optimized pH, and/or protective encapsulation, ensuring consistent potency and efficacy throughout the product's shelf life.

Finally, the inventive uses of these formulations, including their combination with microneedling, low-level light therapy, oral administration of active ingredient(s), and/or adaptogenic or stem cell-derived ingredients, provide synergistic effects that further enhance hair regrowth and scalp health. These combinations yield robust results, the magnitude of improvement is observed when these modalities are used together, and are surprising and unexpected.

Collectively, these advantages and unexpected results position the inventive formulations as a substantial advancement over existing hair regrowth therapies, offering improved efficacy, safety, stability, and/or patient satisfaction

### DETAILED DESCRIPTION OF THE INVENTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “active pharmaceutical ingredient” refers to any chemical compound, biological agent, or substance included in a topical composition whose intended function is to produce a pharmacological, therapeutic, or preventive effect on hair growth, hair loss, or scalp health following administration. This includes any drug, hormone, peptide, or bioactive material that directly influences hair follicle biology, modulates molecular pathways involved in hair cycling, or treats conditions associated with alopecia, irrespective of physical form, concentration, or regulatory status.

The term “finasteride” refers to the compound having the IUPAC name (5α,17β)-N-(1,1-dimethylethyl)-3-oxo-4-azaandrost-1-ene-17-carboxamide; molecular formula C₂₃H₃₆N₂O₂; CAS number 98319-26-7; and molar mass 372.55 g mol⁻¹. Finasteride is a 5α-reductase inhibitor that reduces dihydrotestosterone (DHT) levels to treat androgenetic alopecia. The FDA unique ingredient identifier (UNII) is X0W47C2X83. In topical formulations, it is utilized at 0.1-2.5 wt. % w/w to minimize systemic absorption while maintaining follicular efficacy.

The term “minoxidil” refers to the compound having the IUPAC name 6-(1-piperidinyl)-2,4-pyrimidinediamine 3-oxide; molecular formula C₉H₁₅N₅O; CAS number 38304-91-5; and molar mass 209.25 g mol⁻¹. Minoxidil acts as a potassium channel opener to stimulate hair follicle growth, with a topical concentration range of 2-8% w/w. Its UNII is 5965120SH1, and it complies with USP monographs for purity and stability.

The term “retinoic acid” refers to the compound having the IUPAC name (2E,4E,6E,8E)-3,7-dimethyl-9-(2,6,6-trimethylcyclohex-1-en-1-yl)nona-2,4,6,8-tetraenoic acid; molecular formula C₂₀H₂₈O₂; CAS number 302-79-4; and molar mass 300.44 g mol⁻¹. Retinoic acid enhances epithelial cell turnover and follicular penetration of actives. Topical compositions contain 0.01-0.1% w/w to balance efficacy and irritation potential (UNII. 5688UTC01R).

The term “hydrocortisone” refers to the glucocorticoid with IUPAC name (8S,9S,10R,11S,13S,14S,17R)-11,17-dihydroxy-17-(2-hydroxyacetyl)-10,13-dimethyl-2,6,7,8,9,11,12,14,15,16-decahydro-1H-cyclopenta[a]phenanthren-3-one; molecular formula C₂₁H₃₀O₅; CAS number 50-23-7; and molar mass 362.46 g mol⁻¹. It reduces scalp inflammation at 0.1-1% w/w (UNII: WI4X0X7BPJ) and complies with USP/Ph.Eur. standards for topical use.

The term “aloe vera” refers to the mucilage extracted from Aloe barbadensis leaves, CAS number 85507-69-3, with UNII ZY81Z83H1X. It contains polysaccharides, enzymes, and antioxidants that provide moisturizing and anti-inflammatory effects. Topical formulations incorporate 0.5-2% w/w to enhance skin compatibility and reduce irritation from active ingredients.

The term “propylene glycol” refers to the diol with IUPAC name propane-1,2-diol; molecular formula C₃H₈O₂; CAS number 57-55-6; and molar mass 76.10 g mol⁻¹. This USP/Ph.Eur.-compliant excipient functions as a humectant and solvent, typically included at 5-15% w/w to improve active ingredient stability and follicular delivery (UNII: 6DC9Q167V3).

The term “ethyl alcohol” refers to ethanol, with IUPAC name C₂H₅OH; molecular formula C₂H₆O; CAS number 64-17-5; and molar mass 46.07 g mol⁻¹. It acts as a solvent and antimicrobial agent in topical formulations, used at 20-70% w/w to enhance penetration while maintaining product sterility (UNII: 3K9958V90M).

The term “dutasteride” refers to the dual 5α-reductase inhibitor with IUPAC name (5α,17β)-N-{2,5-bis(trifluoromethyl)phenyl}-3-oxo-4-azaandrost-1-ene-17-carboxamide; molecular formula C₂₇H₃₀F₆N₂O₂; CAS number 164656-23-9; and molar mass 528.54 g mol⁻¹. Topical compositions contain 0.01-0.3% w/w to suppress DHT synthesis (UNII: O0J6ZJ6LLM).

The term “latanoprost” refers to the prostaglandin analog with IUPAC name isopropyl (Z)-7-[(1R,2R,3R,5S)-3,5-dihydroxy-2-[(3R)-3-hydroxy-5-phenylpentyl]cyclopentyl]hept-5-enoate; molecular formula C₂₆H₄₀O₅; CAS number 130209-82-4; and molar mass 432.59 g mol⁻¹. It prolongs the anagen phase at 0.005-0.1% w/w in topical formulations (UNII: 6Z5B6HVF6O).

The term “purified water” refers to water complying with USP/Ph.Eur. specifications (CAS 7732-18-5, UNII 059QF0KO0R). It serves as the primary solvent in topical compositions, constituting 30-70% w/w to adjust final weight and ensure ingredient compatibility.

The term “saw palmetto” refers to the extract from Serenoa repens berries (CAS 90106-88-0, UNII 3Y8H84I8QY). Rich in fatty acids, it inhibits 5α-reductase activity and is included at 1-10% w/w in topical formulations to reduce follicular miniaturization.

The term “biotin” refers to the compound with IUPAC name 5-[(3aS,4S,6aR)-2-oxohexahydro-1H-thieno[3,4-d]imidazol-4-yl]pentanoic acid; molecular formula C₁₀H₁₆N₂O₃S; CAS number 58-85-5; and molar mass 244.31 g mol⁻¹. It strengthens hair keratin at 0.01-0.05% w/w (UNII: 6SO6U10H04).

The term “marine collagen” refers to hydrolyzed collagen peptides derived from marine sources (CAS 9007-34-5). It improves hair tensile strength and is incorporated at 1-5% w/w in topical formulations. UNII varies by source (e.g., 5LFD6G6X6H).

The term “ashwagandha” refers to the extract from Withania somnifera roots (CAS 90106-88-0, UNII 0834K7SS3H). It reduces stress-induced hair loss and is included at 1-5% w/w in topical compositions.

The term “tocotrienols” refer to vitamin E analogs, including delta-tocotrienol (CAS 58864-81-6, UNII 003Y9006QN). These antioxidants reduce oxidative stress in hair follicles at 0.1-1% w/w.

The term “zinc pyrithione” refers to the compound with molecular formula C₁₀H₈N₂O₂S₂Zn; CAS number 13463-41-7; and molar mass 317.70 g mol⁻¹ (UNII: R596H2102T). It functions as an anti-dandruff agent at 0.1-2% w/w, reducing follicular inflammation.

The term “ascorbic acid” refers to the antioxidant with IUPAC name 2-oxo-L-threo-hexono-1,4-lactone-2,3-enediol; molecular formula CH₈H₈O₆; CAS number 50-81-7; and molar mass 176.12 g mol⁻¹ (UNII: PQ6CK8PD0R). Topical formulations contain 0.1-10% w/w to enhance collagen synthesis.

The term “flax seed extract” refers to the extract from Linum usitatissimum seeds (CAS 8001-26-1, UNII 5RZK2P8N3U). Rich in alpha-linolenic acid, it reduces scalp inflammation at 1-5% w/w.

The term “SANDALORE™” refers to the synthetic fragrance compound with IUPAC name 3-methyl-5-(2,2,3-trimethylcyclopent-3-en-1-yl)pentan-2-ol; molecular formula C₁₄H₂₆O; CAS number 65113-99-7; and molar mass 210.36 g mol⁻¹. It is included at 0.01-0.1% w/w to enhance product aesthetics.

The term “caffeine” refers to the compound with IUPAC name 1,3,7-trimethylpurine-2,6-dione; molecular formula C₈H₁₀N₄O₂; CAS number 58-08-2; and molar mass 194.19 g mol⁻¹ (UNII: 3G6A5W338E). It stimulates hair follicle metabolism at 0.1-5% w/w in topical formulations.

The term “lauric acid” refers to the saturated fatty acid with IUPAC name dodecanoic acid; molecular formula C₁₂H₂₄O₂; CAS number 143-07-7; and molar mass 200.32 g mol⁻¹ (UNII: 1160N9NU9U). It stabilizes formulations and exhibits antimicrobial activity at 0.1-2% w/w.

The term “niacinamide” refers to the compound with IUPAC name pyridine-3-carboxamide; molecular formula CH₆N₂O; CAS number 98-92-0; and molar mass 122.12 g mol⁻¹ (UNII: 25X51I8RD4). It improves scalp barrier function and reduces sebum production at 1-5% w/w.

The term “drug substance” refers to the active pharmaceutical ingredient (API) in a medication, such as finasteride, minoxidil, dutasteride, and/or latanoprost, that provides the intended pharmacological or therapeutic effect. It is the pure chemical compound or biologic material that directly contributes to the diagnosis, cure, mitigation, treatment, or prevention of disease, or affects the structure or function of the body.

The term “drug product” refers to the finished dosage form of a medication, such as the topical composition described herein, that contains one or more drug substances along with any necessary inactive ingredients (excipients), and is ready for administration to patients.

The term “particle-size distribution” or “PSD” refers to a list of values or a mathematical function that defines the relative amount, typically by mass, of particles present according to size. For example, the mass-median-diameter (MMD) (expressed as, e.g., d10, d50, d90, etc.) refers to the log-normal distribution mass median diameter. The MMD is considered to be the average particle diameter by mass. The particle size distribution can be obtained by, e.g., laser diffraction, analytical sieving, light scattering, etc. The particle-size distribution can be measured on the drug substance or the drug product. Typically, the particle size distribution will be measured on the drug product when the drug substance is not soluble in the drug product.

Particle size Distribution D10 (or d10) is also written as X10, D(0,1) or X(0,1). It represents the particle diameter corresponding to 10% cumulative (from 0 to 100%) undersize particle size distribution. In other words, if particle size D10 is 7.8 um, then 10% of the particles in the tested sample are smaller than 7.8 micrometer, or the percentage of particles smaller than 7.8 micrometer is 10%. D10 is a typical point in particle size distribution analysis. D10 is also divided into Dv10, Dw10 and Dn10. Dv10 means volume D10, whereas Dw10 is mass D10 and Dn10 is number D10. The particle size distribution D10 (or d10) can be measured on the drug substance or the drug product. Typically, the particle size distribution D10 (or d10) will be measured on the drug product when the drug substance is not soluble in the drug product. Particle size Distribution D50 (or d50) is also written as X50, D(0,5) or X(0,5). It represents the particle diameter corresponding to 50% cumulative (from 0 to 100%) undersize particle size distribution. In other words, if particle size D50 is 7.8 um, then 50% of the particles in the tested sample are smaller than 7.8 micrometer, or the percentage of particles smaller than 7.8 micrometer is 50%. D50 is a typical point in particle size distribution analysis. D50 is also divided into Dv50, Dw50 and Dn50. Dv50 means volume D50, whereas Dw50 is mass D50 and Dn50 is number D50. The particle size distribution D50 (or d50) can be measured on the drug substance or the drug product. Typically, the particle size distribution D50 (or d50) will be measured on the drug product when the drug substance is not soluble in the drug product.

Particle size Distribution D90 (or d90) is also written as X90, D(0,9) or X(0,9). It represents the particle diameter corresponding to 90% cumulative (from 0 to 100%) undersize particle size distribution. In other words, if particle size D90 is 7.8 um, then 90% of the particles in the tested sample are smaller than 7.8 micrometer, or the percentage of particles smaller than 7.8 micrometer is 90%. D90 is a typical point in particle size distribution analysis. D90 is also divided into Dv90, Dw90 and Dn90. Dv90 means volume D90, whereas Dw90 is mass D90 and Dn90 is number D90. The particle size distribution D90 (or d90) can be measured on the drug substance or the drug product. Typically, the particle size distribution D90 (or d90) will be measured on the drug product when the drug substance is not soluble in the drug product.

For example, the term “formulated as particles having D90 ≤8.5 μm” refers to a composition in which the mass-median diameter of 90% of included solid drug particles, as determined by standard particle size analysis techniques (e.g., laser diffraction), is equal to or less than 8.5 micrometers, ensuring uniform particle size distribution for optimized follicular penetration and bioavailability.

The term “penetration enhancer” refers to any compound, material, or system included in a topical formulation in an amount effective to increase the rate, extent, or efficiency by which an active pharmaceutical ingredient passes through the stratum corneum, epidermis, or scalp into the hair follicle or deeper layers, via chemical, physical, or biological means. Penetration enhancers may function by modifying skin barrier structure, solubilizing APIs, or facilitating translocation, and are not limited by chemical class, structure, or mechanism.

The term “treating” (and its grammatical variants such as “treat,” “treated,” and “treatment”) refers to the administration of an active pharmaceutical ingredient (API), or a unit dosage form comprising the API (e.g., a topical composition), to a subject for the purpose of preventing, delaying the onset of, mitigating, curing, healing, alleviating, relieving, altering the course of, remedying, ameliorating, improving, stabilizing, or otherwise affecting a disease or disorder, or one or more symptoms thereof. The disease or disorder may include, but is not limited to, alopecia, hair loss, hair shedding, reduced hair density or thickness, androgenetic alopecia (male pattern baldness), alopecia areata, autoimmune-related hair loss, female pattern hair loss (FPHL), female scalp hair loss, and/or female pattern alopecia.

The term “topical composition” refers to any pharmaceutical, cosmetic, cosmeceutical, or related formulation, in any physical form—including, but not limited to, liquid, semi-solid, solid, particulate, or multiphase formats—comprising at least one active or inactive ingredient, and intended for direct application to the skin, scalp, or hair for local, regional, transdermal, or cosmetic effect. The term expressly encompasses, without limitation, all dosage and delivery forms known in the art or disclosed herein—including solutions, emulsions, suspensions, gels, foams, creams, ointments, serums, oils, lotions, sprays, patches, hydrogels, balms, sticks, powders, concentrates, patches, pastes, sticks, balms, hydrogel patches, microsphere suspensions, liposome or nanoparticle suspensions, waterless concentrates, anhydrous gels, bioadhesive matrices, films, solids, or any combination or hybrid thereof—regardless of vehicle, packaging, or delivery device. ˜The term “topical composition” refers to any pharmaceutical, cosmetic, cosmeceutical, or related formulation comprising at least one active or inactive ingredient and intended for direct application to the skin, scalp, or hair for local, regional, transdermal, therapeutic, or cosmetic effect. “Topical composition” includes, but is not limited to, all formulations, dosage forms, and delivery vehicles described herein or known in the art, including but not limited to solutions, emulsions, suspensions, gels, foams, creams, ointments, serums, oils, lotions, sprays, patches, hydrogels, balms, sticks, powders, concentrates, films, modular vehicles, bioadhesive matrices, microsphere systems, liposome carriers, nanoparticle suspensions, hydrogel patches, waterless concentrates, and any formulations or systems suitable for topical or transdermal delivery of actives or excipients, regardless of packaging or delivery device.

The term “shampoo” refers to a liquid, semi-solid, gel, or foaming composition containing one or more surfactants designed for cleansing the scalp and hair, optionally further comprising active ingredients, conditioners, thickeners, fragrances, or therapeutic agents intended to remove sebum, dirt, pollutants, microorganisms, or styling products, while delivering additional benefit to scalp or hair.

The term “solution” or “topical solution” refers to ˜a homogeneous, single-phase liquid formulation in which one or more solutes (active and/or inactive ingredients) are molecularly or ionically dissolved in one or more solvents such as water, alcohol(s), propylene glycol, or combinations, typically used for topical application to the skin or scalp. Solutions may include additional excipients to optimize stability, penetration, or user sensory experience. ˜The term “solution” or “topical solution” refers to a homogeneous, single-phase liquid formulation in which one or more active or inactive ingredients are dissolved in one or more solvents, and which is intended for topical application to the skin, scalp, or hair. “Solution” includes compositions with pharmaceutical or cosmetic effect, optionally containing excipients, carriers, penetration enhancers, stabilizers, or other additives as described or known in the art.

The term “oil” or “topical oil” refers to a hydrophobic, non-aqueous liquid, semi-solid, or viscous composition containing at least one lipid-based component, which may be of natural, mineral, or synthetic origin, and is formulated for topical application. Oils may act as solvents, penetration enhancers, emollients, or carriers for actives, and may be used alone or as a part of more complex formulations.

The term “serum” or “topical serum” refers to a water-based, hydroalcoholic, or emulsion composition of low to moderate viscosity, typically formulated with a high concentration of one or more active ingredients, humectants, or conditioners, designed for direct application to the scalp or hair to deliver targeted therapeutic or cosmetic benefits, and which may be rapidly absorbed without leaving residue.

The term “foam” or “topical foam” refers to a composition, in liquid or semi-solid form, that dispenses as a froth comprised of gas bubbles dispersed within a liquid or semi-solid matrix, created via propellant, surfactant, or mechanical aeration. Foams may enhance spreading, skin penetration, cosmetic elegance, or ease of scalp application.

The term “cream” or “topical cream” refers to a semi-solid emulsion formulation, typically oil-in-water (O/W) or water-in-oil (W/O), containing emulsifiers, actives, and excipients, possessing a soft, spreadable consistency suitable for topical application. Creams are formulated to enhance absorption, provide moisturization, and permit uniform distribution of actives across the skin or scalp.

The term “gel” or “topical gel” refers to ˜a semi-solid, colloidal system comprising a network of polymers, gelling agents, or thickeners that trap a substantial portion of a liquid phase (such as water, alcohol, or oil), resulting in a clear or translucent structure with defined shape, elasticity, and spreadability, and suited for topical delivery of actives to the scalp or skin. ˜The term “gel” or “topical gel” refers to a semi-solid or viscous formulation containing a network of polymeric, gelling, or thickening agents that immobilizes a liquid phase (including, but not limited to, water, alcohols, or oils), forming a composition suitable for topical application to the skin, scalp, or hair. “Gel” includes all hydrogel, organogel, or multiphase gel systems designed to deliver active or inactive ingredients, with or without additional excipients.

The term “gel patch” or “topical gel patch” refers to a flexible, adhesive or semi-adhesive device incorporating a gel matrix (hydrogel, organogel, or combination) that contains one or more active ingredients for sustained or controlled release to the scalp or skin, with conformability and adherence to curved surfaces, optionally providing a moisturizing, cooling, or occlusive effect.

The term “lotion” or “topical lotion” refers to a flowable, low- to medium-viscosity emulsion (oil-in-water or water-in-oil) or solution for topical application to the scalp or skin, formulated to deliver active and/or conditioning ingredients in a spreadable, rapidly absorbing format, and may include humectants, emollients, or stabilizers as described herein.

The term “emulsion” or “topical emulsion” refers to a two-phase liquid system comprising discrete droplets of one immiscible liquid (e.g., oil) dispersed throughout a continuous phase of another (e.g., water), stabilized by emulsifying agents, and optionally containing actives or excipients for topical administration.

The term “ointment” or “topical ointment” refers to a semi-solid topical composition with a hydrophobic, oily, or waxy base, typically free of or containing minimal water, formulated for prolonged retention and occlusive delivery of active ingredients to the skin or scalp, providing enhanced moisture barrier and potential for increased penetration of the API(s).

The term “paste” or “topical paste” refers to a semi-solid, viscous formulation containing a high proportion of solids (such as powders, actives, or absorbents) dispersed in a lipid, aqueous, or hydroalcoholic base, designed for topical application at the scalp or skin, potentially providing protective, occlusive, or controlled-release effects.

The term “spray” or “topical spray” refers to a liquid or semi-liquid formulation that is applied to the scalp or skin as a fine mist or droplets via a pump, aerosol, or pressurized system, and which may comprise solution, suspension, or emulsion vehicles to deliver active or inactive ingredients.

The term “hydrogel” or “topical hydrogel” refers to a gel-based formulation wherein water is the principal liquid phase, polymerized or cross-linked to form a three-dimensional network, producing a soft, moisture-rich, and often transparent system suitable for delivering actives, maintaining skin hydration, and optionally providing cooling or soothing effects upon application.

The term “balm” or “topical balm” refers to a semi-solid or solid topical preparation composed primarily of lipids, waxes, butters, or related materials, which may include one or more active or inactive ingredients, and is formulated to provide occlusion, conditioning, or protection as well as delivery of the actives to scalp or skin.

The term “stick” or “topical stick” refers to a solid, cylindrical, or other shaped dosage form designed for direct topical application to the skin or scalp, comprising a wax-based, lipid-based, or polymeric matrix that incorporates one or more actives, allowing convenient and targeted administration without the need for the user to touch the formulation itself.

The term “patch” or “topical patch” refers to a solid, flexible, or semi-flexible device that adheres to the scalp or skin and delivers active ingredients over time, which may include reservoirs, matrices, or arrays (such as microneedle arrays, gel patches, or hydrogel patches) for controlled, sustained, or direct localized delivery of actives, optionally containing penetration enhancers or adhesives.

The term “waterless concentrate” or “topical waterless concentrate” refers to a topical formulation in which water is substantially or entirely absent prior to use, typically formulated as anhydrous gels, powders, or oils, designed to be diluted, reconstituted, or applied directly, and containing concentrated levels of actives, carriers, or excipients for increased potency, extended shelf-life, or reduced microbial risk.

The term “anhydrous gel” or “topical anhydrous gel” refers to a gel-based composition where the primary liquid or continuous phase is non-aqueous (e.g., oils, silicones, polyols, or other hydrophobic/hydroalcoholic carriers), containing structuring agents that produce a gelled, spreadable matrix for the delivery of actives without the presence of free water.

The term “bioadhesive matrix” or “topical bioadhesive matrix” refers to any topical formulation or material—such as a gel, hydrogel, film, patch, or polymeric system—specifically designed to adhere to the skin or scalp for prolonged residence time, improved localization, controlled or sustained release of active ingredients, and enhanced scalp or follicular penetration efficiency. Typical bioadhesive polymers include chitosan, hyaluronic acid, polyacrylates, polyvinylpyrrolidone (PVP), carbomer, cellulose derivatives (e.g., hydroxyethylcellulose), gelatin, alginate, pectin, and their blends or copolymers. The degree and durability of bioadhesion is quantified using validated in vitro protocols such as texture analyzer force measurement (e.g., TA.XTplus), peel or detachment tests (ASTM F2258-05), or ex vivo retention studies using excised porcine or human skin, with residence time, tack force, or detachment energy as key output metrics.

A “bioadhesive hydrogel” is a subset of “bioadhesive matrix.” The term “bioadhesive hydrogel” refers to a semi-solid, polymer-based matrix containing water and one or more bioadhesive agents (e.g., chitosan, hyaluronic acid), which is formulated to adhere to the skin or scalp upon topical application, thereby prolonging localization at the target site and providing controlled, sustained release of active ingredients for enhanced and targeted follicular delivery.

Bioadhesive polymers suitable for use in topical hair regrowth compositions described herein include both natural and synthetic classes, selected for their proven ability to adhere to the scalp, prolong residence time, enhance cosmetic acceptability, and enable efficient follicular delivery of active ingredients. Commonly used natural bioadhesives encompass chitosan-a deacetylated derivative of chitin valued for its biocompatibility and robust scalp adhesion; hyaluronic acid, for its moisture retention and bioadhesive properties; alginate, a polysaccharide from brown seaweed with excellent gelation and ion-responsive crosslinking; gelatin, contributing reversible, temperature-sensitive crosslinking; and pectin, a fruit-derived polysaccharide used for modulating release kinetics. Other examples are cellulose derivatives such as hydroxyethylcellulose, carboxymethylcellulose (CMC), and hydroxypropyl methylcellulose (HPMC), which contribute both bioadhesive and rheology-modifying properties. Among synthetic classes, polyvinylpyrrolidone (PVP); poly(acrylic acid) and its crosslinked version carbomer (notably Carbopol); sodium polyacrylate; and polyvinyl alcohol (PVA) are frequently employed due to tunable viscosity, safety profile, and strong adhesion under moist conditions. Further options include polyquaternium-10 (a mild cationic derivative with conditioning and bioadhesive traits); poloxamers (such as Pluronic F127), capable of forming thermoresponsive gels; as well as smart-responsive hydrogel polymers, including block copolymers and functionalized polyacrylates, which modulate bioadhesion or release in response to external stimuli (e.g., temperature, moisture, pH). Each of these polymers may be used singly or in combination, optimized according to targeted physical form (gel, matrix, patch), compatibility with incorporated actives, regulatory status, and for delivering desired release profiles and optimized user sensory experiences in advanced topical hair regrowth therapies.

The term “smart-responsive polymer matrix” refers to a polymeric carrier system in which physical or chemical properties, such as viscosity, permeability, or release kinetics, dynamically change in response to external stimuli (e.g., temperature, pH, humidity, or scalp condition), thereby controlling the timing, amount, or pattern of active pharmaceutical ingredient delivery for personalized therapy.

The term “microsphere suspension” or “topical microsphere suspension” refers to a liquid or semi-solid formulation containing suspended microspheres—solid, porous, or polymer-based spherical particles (1-1000 μm diameter) loaded with actives and/or excipients—for controlled, sustained, or targeted release of the APIs when applied topically.

The term “liposome or nanoparticle suspension” or “topical liposome or nanoparticle suspension” refers to a topical formulation comprising dispersed lipid vesicles (liposomes, typically 50-5000 nm in diameter) or solid or colloidal nanoparticles (e.g., solid lipid nanoparticles, polymeric nanoparticles, metallic nanoparticles, typically 1-1000 nm in diameter), encapsulating or carrying active ingredients, wherein such particles enhance delivery, penetration, stability, or controlled release of the APIs upon topical administration.

The term “nanoparticle/liposome suspension” refers to a topical composition wherein one or more active pharmaceutical ingredients are encapsulated within or bound to nanometer-scale particles or lipid-based vesicles (liposomes), suspended in a liquid or semi-solid vehicle, to enhance penetration, stability, and controlled release for targeted follicular delivery.

The term “thermoresponsive gel” refers to a topical or transdermal gel formulation whose viscosity, phase, or physical state changes in response to temperature, typically becoming less viscous or transitioning from semi-solid to liquid upon warming to skin or scalp temperature, thereby facilitating enhanced or on-demand release or penetration of active ingredient(s).

The term “phase-change gel” refers to a formulation or delivery vehicle that undergoes a reversible transition in physical phase (e.g., from liquid to semi-solid or solid) as a result of external stimuli such as heat, pH, or mechanical manipulation, designed to optimize user application, scalp retention, or active release profiles.

The term “slow-release gel” refers to a gel-based matrix designed to provide extended, controlled, or sustained release of active ingredient(s) upon topical application, enabling prolonged scalp contact and gradual absorption into targeted scalp or follicular sites.

The term “solid lipid nanoparticle” refers to a colloidal carrier system composed of solid-phase lipid particles typically ranging from 50 to 1000 nanometers in diameter, loaded with one or more active or inactive agents, and used to enhance follicular delivery, penetration, stability, or sustained release in topical formulations.

The term “hydroalcoholic vehicle” refers to a carrier or solvent system for a topical formulation, comprising both water and one or more alcohols (such as ethanol, isopropanol, or propylene glycol), which may be combined in various proportions to optimize solubility, stability, penetration, or cosmetic elegance.

The term “patch” refers to any flexible, adhesive or semi-adhesive topical dosage form containing one or more ingredients for sustained, controlled, or targeted release into or onto the scalp over a defined period, optionally including device features such as microneedle arrays or sensors.

The term “microneedle array” refers to a device or patch system for topical or transdermal delivery that incorporates multiple microscopic needles or projections, composed of dissolvable, biodegradable, or inert materials, arranged in a defined pattern, and intended to create microchannels in the stratum corneum to enhance penetration and direct deposition of active ingredients.

The term “follicle-priming agent” refers to a compound or mixture, applied sequentially or with a main active regimen, that is designed to condition, sensitize, or temporarily modify the scalp or pilosebaceous unit to increase the subsequent uptake, penetration, or biological effect of a subsequently administered active ingredient.

The term “pharmaceutical-grade container” refers to any packaging, bottle, vial, ampoule, or dispenser composed of materials and manufactured under standards meeting regulatory requirements for medical or pharmaceutical use, ensuring chemical and physical compatibility, stability, and integrity of topical compositions.

The term “energy-activated release” refers to a topical delivery system or composition wherein release, activation, or bioavailability of active ingredients is triggered or enhanced by specific external energy sources. These non-limiting energy sources include: (1) Phototherapy: Exposure to defined wavelengths of light, usually in the red/infrared spectrum (e.g., 630-830 nm, delivered by continuous or pulsed laser or LED), which may activate carrier polymers, photoactivatable compounds, or improve dermal penetration; (2) Microcurrent: Low-amplitude electrical stimulation (typically 0.5-2.0 mA) delivered via comb, patch, or wearable device to modulate permeability or trigger release mechanisms; (3) Ultrasound: Application of focused acoustic energy (e.g., 1-3 MHz) for sonophoresis, enabling penetration or release of actives from encapsulated systems; (4) Thermal/Heat-Activated: Self-heating packs, phase-change gels, or external warming devices that cause physical or chemical transitions leading to enhanced active release at defined temperature thresholds (e.g., ≥32-40° C. for gel or polymer transition). These modalities may operate independently or in combination, and the definition covers any present or future energy-activated mechanism relevant to topical pharmaceutical and cosmeceutical delivery.

The term “bioactive aroma compound” refers to a volatile or non-volatile compound included in a topical formulation not solely for sensory purposes, but also posited or demonstrated to influence physiology, follicular biology, user compliance, mood, or associated biological pathways via olfactory or other molecular mechanisms.

The term “pH-modifying buffer system” refers to a set of components in a topical formulation, present in sufficient quantity to maintain or adjust the pH of the vehicle during storage or upon application, thereby affecting stability, solubility, penetration, or activation of actives.

The term “content uniformity” refers to the degree to which a pharmaceutical or cosmetic preparation contains the same amount of active ingredient(s) per unit or aliquot, within prescribed limits, as assessed by validated analytical techniques (e.g., HPLC), thus ensuring dose reproducibility and product reliability.

The term “batch-to-batch consistency” refers to the reproducibility of manufacturing and final product characteristics, including active content, physical properties, and performance metrics, as measured quantitatively (such as using relative standard deviation, RSD) across multiple production batches of a given formulation.

The term “yield stress” refers to the minimum applied stress at which a topical composition or gel begins to flow as a liquid rather than behave as an elastic or viscoelastic solid, relevant to controlled dispensing and application properties.

The term “thixotropic index” refers to the ratio or relationship between the apparent viscosity of a topical composition or gel measured at low versus high shear rates, which reflects the composition's ability to decrease its viscosity under shear and recover its structure at rest, impacting spreadability and ease-of-use.

The term “span value” refers to a dimensionless metric (typically (D90−D10)/D50) derived from particle size distribution data, quantifying the width or range of the particle size distribution within a topical or particulate formulation and used as a measure of formulation uniformity and performance potential.

The term “Franz cell diffusion assay” refers to an in vitro analytical technique that evaluates the rate, extent, or kinetics of topical ingredient penetration (across skin or membrane), typically using a donor chamber (containing the formulation) and a receptor chamber (collecting transmigrated material), separated by an excised skin or synthetic membrane barrier.

The term “scalp diagnostic feedback system” refers to any integrated or companion device, wearable, sensor, or analytical tool associated with a topical regimen, designed to monitor scalp parameters (such as pH, hydration, sebum, inflammation, or temperature) and provide feedback or data to optimize, personalize, or modulate the treatment protocol.

The term “exosome” refers to a membrane-bound, nanometer-scale vesicle (typically 30-200 nm) derived from cells, particularly relevant in the context of stem cell- or mesenchymal cell-derived conditioned media, and included in topical formulations to promote follicular regeneration, signaling, or anti-inflammatory effects.

The term “stem cell conditioned media” refers to a sterile fluid or reconstituted material containing extracellular factors, growth signals, cytokines, or vesicles (e.g., exosomes), obtained from culturing stem cells under defined conditions, and used in topical formulations to support or augment follicular regeneration or hair growth.

The term “regenerated bioactive scaffold” refers to a matrix, film, or structure formed in situ on the scalp following topical application, comprising biocompatible and/or biodegradable material(s), optionally loaded with actives or biological cues, which is designed to both physically support follicle structures and provide sustained release or microenvironmental conditioning for follicle recovery.

The term “multi-chambered packaging” refers to any container, device, or dispensing system comprising two or more physically separate compartments, chambers, or reservoirs, each independently containing a formulation, active or inactive ingredient, excipient, or composition. Such packaging is configured to enable separate storage of individual components and permits dispensing—whether simultaneous, sequential, combined, or user-selected—at the time of use. Multi-chambered packaging supports customizable, stepwise, or programmable topical administration, and encompasses any design, layout, or mechanism wherein the physical boundaries or barriers maintain ingredient segregation until dispensing or mixing is desired by the user or operator. This includes, but is not limited to, dual-chamber bottles, pump dispensers, snap-together vials, blisters, sachets, syringes, or any equivalent system that preserves stability and permits flexible delivery of individual or mixed components.

The term “sequential dispensing packaging” refers to a packaging system constructed to deliver two or more compositions or phases either one after another, in a defined or programmable sequence, thus enabling multi-step topical treatment protocols or physically separated actives that are combined only at the point of use.

The term “personalized regimen scheduling” refers to any dosing, application, or administration schedule that is tailored or modifiable based on individual patient characteristics, feedback, scalp responses, measured outcomes, or preferences, and which may be facilitated by digital, app-based, or device-based guidance.

The term “wearable device” refers to an article of apparel or accessory that is configured to be worn on the body or scalp and that incorporates one or more sensors, feedback systems, or active delivery or diagnostic elements for monitoring, controlling, or optimizing the topical hair growth regimen.

The term “machine learning algorithm” refers to a computational method or system that utilizes data inputs, historical outcomes, or live feedback to identify patterns or optimize, recommend, or automate adjustments to topical hair growth regimen variables, including dosing, combinations, or scheduling, either on-device or via cloud/app.

The term “Bluetooth-enabled dispenser” refers to a topical dispensing apparatus that incorporates wireless communication functionality allowing data transmission to or from a digital device (e.g., smartphone, tablet, computer) for the purposes of tracking, programming, logging, or automating usage of the topical composition.

The term “relative standard deviation (RSD)” refers to a statistical measure of variability expressed as the standard deviation divided by the mean, typically presented as a percentage, and used, for example, to quantify batch-to-batch consistency, content uniformity, or physical property reproducibility in topical formulation manufacturing.

The term “chemical stability” refers to the retention of potency, molecular integrity, and/or physical properties of an active ingredient or formulation under specified storage, handling, or use conditions and timeframes; including, but not limited to, resistance to significant degradation, precipitation, color change, or loss of efficacy.

The term “accelerated stability testing” refers to a protocol in which a formulation or ingredient is stored under elevated temperature and/or humidity for a specified time, in order to predict or estimate shelf-life, chemical integrity, or stability under normal conditions, as per regulatory guidelines.

The term “anagen phase” refers to the growth phase of the hair cycle, during which follicles are active, hair shafts elongate, and the density and caliber of terminal hairs increase, measurable by trichogram, trichoscopy, or histological or molecular methods.

The term “anagen-to-telogen ratio” refers to the proportion of hair follicles in the active (anagen) phase versus those in the resting (telogen) phase, as determined by scalp analysis techniques; an indicator of hair cycle health and hair regrowth activity.

The term “trichoscopy” refers to a non-invasive scalp imaging and diagnostic technique using high-magnification videodermoscopy or digital microscopy to assess, quantify, or monitor hair and scalp parameters, such as density, diameter, ratio of terminal to vellus hairs, and scalp health.

The term “erythema score” refers to a quantitative or semi-quantitative measure of skin redness or irritation, typically graded by trained observers or using a defined visual or instrumental scale, to evaluate local tolerability of topical compositions.

The term “low-level light therapy (LLLT)” refers to the application of coherent or non-coherent light in the visible red or near-infrared spectrum (typically 600-900 nm), delivered via laser, LED, or similar devices, to the scalp or hair-bearing areas for the purpose of promoting hair follicle activity or regrowth, as part of or adjunct to the described hair regrowth regimen.

The term “microneedling” refers to the method or process of causing controlled, reproducible micro-injuries or punctures to the scalp using a device (e.g., roller, stamp, or array), typically with needles in the range of 0.1-1.5 mm, to enhance absorption and efficacy of topically applied formulations and/or stimulate follicular activity.

The term “auto-blending technology” refers to a system, device, or method that combines or mixes two or more liquid, semi-solid, or particulate ingredients, excipients, or formulations at the time of use, enabling customizable or user-specific topical dose ratios or regimens.

The term “antioxidant system” refers to one or more compounds included in a topical formulation for the primary purpose of inhibiting or reducing oxidative degradation of actives, excipients, or formulation matrices, thereby increasing shelf-life, potency, and/or scalp compatibility (e.g., BHT, EDTA, tocopherols, ascorbic acid).

The term “stabilizer” refers to an agent or combination added to a topical formulation to improve or maintain physicochemical stability, potency, formulation uniformity, or shelf-life; examples include antioxidants, chelators, surfactants, preservatives, and pH adjusters.

The term “hair fiber cosmetic additive” refers to any solid, particulate, or powder ingredient, such as keratin fibers, fibers of synthetic or natural origin, or temporary volumizing materials, that is incorporated into or co-administered with a hair growth regimen to provide the appearance of increased hair density, volume, or coverage, either immediately or as a complement to regrowth.

The term “environmental shield” refers to a topical formulation or composition that incorporates one or more agents providing physical or chemical protection against environmental stressors such as UV radiation, pollution, oxidative agents, or humidity, thereby safeguarding active ingredients and/or scalp tissues.

The term “field effect” refers to the influence of a localized electric, magnetic, or electromagnetic field generated by a device or applicator on the permeability, absorption, or release profile of a topically applied ingredient or composition.

The term “drying time” refers to the elapsed interval between application of a topical composition to a surface (such as the scalp or skin) and the point at which the composition becomes visibly dry or tactilely non-tacky under ambient or specified conditions, measurable using established analytical protocols (e.g., filter paper transfer, surface resistance methods, or subjective assessment).

The term “device-assisted delivery” refers to any method in which a physical, mechanical, or energy-based device is used in combination with a topical composition to enhance penetration, distribution, uptake, or therapeutic effect of active ingredients. Key examples include: (1) Microneedling: Use of rollers, stamps, or array devices with needle lengths typically 0.1-1.5 mm for creating microchannels through the stratum corneum; (2) Ultrasound: Application of pulsed or continuous ultrasound (e.g., 1-3 MHz) to increase skin permeability and facilitate transdermal delivery; (3) Low-Level Light Therapy (LLLT): Application of coherent or non-coherent light, most commonly in the 630-830 nm wavelength range (true laser or high-power LED) to biologically stimulate hair follicles and improve active uptake. Other modalities include microcurrent stimulation (0.5-2.0 mA), radiofrequency, or thermal activation, as known in the art.

The term “user-tailored modular delivery kits” refers to any kit, system, or set of components designed to permit end-users, caregivers, or healthcare providers to select, mix, assemble, or adjust the composition, dose, or sequence of topical formulations, actives, enhancers, or adjuncts prior to or during administration, thereby individualizing the regimen according to user needs, preferences, or clinical guidance.

The term “regimen scheduling” refers to the defined protocol, time table, or sequence by which topical compositions are administered to the scalp or hair, encompassing frequency, timing, duration, and dosing adjustments, and optionally guided by digital applications, user preference, or clinical feedback.

The term “regimen personalization” refers to the adjustment, modification, or tailoring of the dosing, ingredient combination, timing, concentration, or mode of administration of a topical hair regrowth regimen for an individual user, based on physiologic data, personal preference, feedback, or clinical outcomes, either manually or through device/app-guided protocols.

The term “potency” refers to the chemical or biological strength of an active ingredient within a composition, expressed as the proportion (%) of the initial or labeled amount that remains after mixing, packaging, or storage for a specified duration and under specified conditions.

The term “content uniformity” refers to the consistency of active pharmaceutical ingredient (API) content per unit dose, aliquot, or dispensed volume of a topical formulation, ensuring that each administration delivers a substantially similar amount of API within a defined tolerance, as measured by validated analytical methods such as HPLC or other pharmacopeial standards.

The term “ex vivo follicular diffusion assay” refers to any experimental, analytical, or model-based test conducted outside a living organism, on excised skin, tissue, or synthetic membrane, designed to quantify the diffusion, penetration, or follicular deposition of topically applied ingredients, typically employing donor/receptor chambers and validated measurement protocols.

The term “real-time device validation” refers to the ongoing, contemporaneous monitoring, measurement, or confirmation of product quality, physical parameters, dosing accuracy, or clinical endpoints during manufacturing, dispensing, or use of a topical composition, employing integrated sensors, analytical devices, or digital tracking to ensure compliance with specified standards.

The term “multi-dose applicator” refers to a dispensing device or system capable of delivering multiple, discrete doses of a topical composition, each dose comprising a defined quantity of APIs, excipients, or formulations, with or without sequential or simultaneous release, supporting regimen flexibility and accuracy.

The term “stepwise protocol” refers to any administration regimen, method, or process in which two or more topical compositions, actives, enhancers, or adjunctive agents are applied in a defined sequence, with each step intended to condition, prime, administer, or complete the treatment for enhanced efficacy or reduced side effects.

The term “compartment(s)” refers to any physically or functionally discrete section, chamber, reservoir, or area of a packaging system, bottle, or applicator used to store, segregate, or dispense one or more compositions, APIs, excipients, or formulation phases, prior to or during mixing or administration.

The term “sensor-guided dosing” refers to the adjustment, control, or timing of the administration of a topical composition based on input, measurement, or feedback from one or more sensors—physical, chemical, biometric, or digital—designed to inform, optimize, or personalize the regimen for improved efficacy or safety.

The term “modular components” refers to the separate, adaptable, or interchangeable elements, cartridges, or units of a kit, device, or system that enable selection, assembly, mixing, or sequencing of various formulations, actives, excipients, or devices for customized topical administration.

The term “multi-chambered packaging” refers to any package, bottle, or container configured with two or more discrete chambers, reservoirs, or compartments, each capable of separately storing compositions, APIs, or excipients, enabling sequential, simultaneous, or blended dispensing per user or protocol selection.

The term “sequential dispensing packaging” refers to packaging or dispensing systems constructed to deliver two or more distinct compositions or formulation phases in a predefined, programmable, or user-selected sequence, such that separate agents, priming steps, or actives are administered one after another.

The term “follicular deposition” or “follicular API deposition” refers to the extent or proportion of a topically applied ingredient or active agent that is localized or delivered specifically to hair follicles (versus the stratum corneum or general skin surface), as determined by direct or indirect measurement techniques (e.g., biopsy, imaging, tracer, or Franz cell models).

The term “epidermal retention time” refers to the duration for which an active ingredient or excipient remains detectable within the epidermal layers (excluding or prior to follicular uptake) after topical application, as determined by imaging, sampling, or analytical chemistry methods.

The term “transepidermal water loss (TEWL)” refers to the rate at which water vapor passes from inside the body through the epidermal layer to the surrounding atmosphere, employed as a measure of skin barrier integrity and as a biomarker for evaluating the impact of topical compositions on skin or scalp permeability.

The term “degree of scalp DHT reduction” refers to the measured or estimated decrease in scalp dihydrotestosterone (DHT) levels, expressed as a percentage relative to baseline or control, following the application of a topical composition, assessed via biochemical, immunoassay, or other validated platforms.

The term “hair count increase” refers to the measured change (absolute or percentage) in the number of visible, countable hair shafts (of any or specified diameter) per unit area of the scalp over a specified treatment interval, as determined by trichoscopy, phototrichogram, or direct counting methods.

The term “hair shaft diameter” refers to the linear measure, typically in micrometers (μm) or millimeters (mm), of the cross-sectional width of an individual hair fiber, calculated using microscopic, phototrichogram, or digital imaging analysis protocols.

The term “accelerated stability testing” refers to the storage of a composition or its components under elevated temperature, humidity, light, or other stressors, for the purpose of estimating or modeling its shelf life, degradation profile, or physical/chemical integrity under normal use, following ICH, USP, or comparable guidelines.

The term “API-loaded nanoparticle” refers to any nano-scale particle or carrier system that encapsulates, binds, or associates with an active pharmaceutical ingredient (API), formulated to enhance, modify, or control the delivery, penetration, or release profile of said API on or within the scalp or skin.

The term “multi-API dose personalization” refers to any method, system, kit, or device that enables end-users or prescribers to customize, at the point of use or dispensing, the relative concentrations, proportions, or types of two or more active pharmaceutical ingredients included in a topical regimen.

The term “multi-cartridge applicator” refers to any applicator, dispenser, or administration device that incorporates, houses, or is otherwise configured to receive, hold, and dispense the contents from two or more separate cartridges, reservoirs, or compartments. The cartridges may independently store formulations, actives, excipients, or any combination thereof.

The device is designed to enable mixing, sequential or simultaneous dispensing, and/or programmable release of ingredients, whether prior to, during, or after topical application. The multi-cartridge applicator thus facilitates flexible, individualized, or stepwise administration regimens, supporting point-of-use preparation, dose personalization, or modular therapy combinations, and is not limited by physical format or actuation method.

The term “companion app” refers to a software application, typically for mobile devices, that facilitates, supports, or optimizes the use, compliance, tracking, personalization, or data analysis of the topical hair-regrowth regimen or device described herein.

The term “self-heating pack” refers to a package or container, associated with a topical composition, that contains one or more elements or reagents which generate heat upon activation (e.g., mixing, exposure to air, or mechanical force), designed to increase skin temperature, improve absorption, or enhance user comfort at or near the time of application.

The term “regimen feedback system” refers to any device, app, algorithm, or integrated platform that collects, processes, or responds to user- or scalp-specific real-time or historic data (e.g., adherence, response, adverse effects, biometric readings) and provides guidance, adjustments, or recommendations to optimize the topical treatment.

The term “follicle priming” refers to a preparatory step or protocol prior to application of a main active agent, involving the use of a priming substance, mechanical action, or environmental condition, intended to increase subsequent follicular uptake, efficacy, or penetration of active ingredients.

The term “post-treatment lotion” refers to a composition formulated for application after the main topical treatment, containing one or more agents specifically targeting scalp barrier renewal, hydration, anti-irritation, or conditioning, to improve tolerability, support recovery, or complement the primary regimen.

The term “adverse reaction detection” refers to the function or capability, in a regimen, device, sensor, or system, of identifying, quantifying, or alerting about unintended or undesirable outcomes (e.g., inflammation, irritation, redness) in response to administration of the topical composition.

The term “content adjustment protocol” refers to a defined procedure, whether manual, device-enforced, or digital, for altering the concentrations or absolute amounts of individual ingredients (active or inactive) in a topical formulation prior to or during use, to accommodate personalization, response, or user preference.

The term “modular delivery kit” refers to a system, product, or kit that comprises multiple, physically separate vessels, components, compartments, cartridges, or delivery devices, each individually or collectively containing one or more formulations, actives, excipients, or adjunctive materials. The modular delivery kit is configured to permit end-user configuration, selection, mixing, sequencing, or other customization of the topical regimen, including the ability to adapt dose ratios, ingredient combinations, or treatment protocols to specific user requirements, preferences, or clinical guidance. The kit may support self-administered, physician-directed, or app-guided assembly and use, and includes all permutations enabling at-home or point-of-care preparation, tailored administration, or stepwise regimen construction.

The term “CMC mimic” refers to any ingredient, blend, or system incorporated within a topical composition designed to emulate, supplement, or restore the cell membrane complex (CMC) structure and function in the scalp or hair fiber, including but not limited to ceramides, cholesterol, and fatty acids, and supporting scalp barrier integrity or follicular health. The term “active loading” refers to the process, method, or resultant property of incorporating one or more active pharmaceutical ingredients or actives into a carrier, matrix, or delivery vehicle within a topical composition to achieve a targeted concentration, distribution, or release profile for therapeutic or cosmetic benefit.

The term “additional API” refers to any one or more active pharmaceutical ingredients, drugs, or bioactives that are included in a topical composition in addition to a primary active, and may support, augment, or modify the intended therapeutic effect or regimen.

The term “adherent matrix” refers to any physical or chemical structure, formulation, or polymeric network within a topical composition that is designed to adhere to the scalp or hair surface for prolonged residence time, controlled release of actives, or improved delivery efficiency.

The term “batch uniformity” refers to the property or measure by which individual units, lots, or batches of a topical composition or kit demonstrate consistency with respect to active content, physical properties, or performance characteristics, typically established by statistical analysis and validated testing protocols.

The term “biodegradable polymer” refers to any polymeric material included in a topical or delivery system that is capable of being broken down by biological processes or environmental conditions, contributing to controlled release, scaffold formation, or matrix dissolution, with minimal residue or toxicity.

The term “botanical complex” refers to any blend, mixture, or proprietary preparation of botanical extracts, oils, or actives included in the topical composition, formulated to impart synergistic, additive, or complementary effects supporting scalp or hair health, regrowth, or other performance attributes.

The term “botanical extract” refers to any concentrated or isolated substance obtained from plant, herb, seed, or similar sources, containing bioactive compounds or nutrients and included in the topical composition for cosmetic, therapeutic, or supportive function.

The term “calibrated dropper” refers to a dispensing component, typically included as part of a topical kit or packaging, that is designed to accurately administer a defined volume of the composition per actuation; calibration may be to ±0.05 mL for precise dosing.

The term “color stabilizer” refers to any ingredient or system incorporated to minimize, prevent, or compensate for color change, oxidation, or photodegradation over time within the topical composition, preserving visual consistency and integrity.

The term “color-matched hair fiber” refers to any particulate or powder-based additive, typically consisting of keratin or similar fiber material, formulated in shades matching human hair, and included to provide immediate cosmetic coverage or volumization alongside regrowth therapy.

The term “colorimetric strip” refers to any paper, film, or sensor device integrated with or used alongside the topical regimen to visually indicate the presence, absence, or level of specific scalp parameters (e.g. pH, inflammation, moisture) for feedback or optimization purposes.

The term “compliance booster” refers to any ingredient, system, or feature incorporated in the topical composition, kit, or regimen that serves to improve patient or user adherence, usage regularity, or satisfaction, such as sensory modifiers, aroma compounds, or feedback mechanisms.

The term “core formulation” refers to any base, prototype, or foundational recipe for a topical composition comprising essential actives, excipients, and carriers, which may be modified, customized, or built into multiple commercial SKUs or variants.

The term “cosmetic adjuvant” refers to any non-active additive, excipient, or ingredient included in the topical composition to enhance physical stability, appearance, sensory properties, or cosmetic benefits without imparting primary therapeutic effect.

The term “dose personalization” refers to any method, system, or feature allowing adjustment of the absolute or relative dose(s), concentration(s), or ratio(s) of active ingredients within a topical regimen or device, tailored based on user characteristics, physician guidance, or feedback data.

The term “electrostatically charged” refers to the property or method by which fibers, powders, or particulate additives within the topical composition acquire or retain an electric charge, typically to increase adherence to scalp or hair, improve cosmetic covering, or facilitate application.

The term “ex vivo assay” refers to any analytical, experimental, or test protocol performed on biological tissue, cell, or system outside of a living organism, such as Franz cell diffusion or tissue penetration assessment, to quantify or measure parameters relevant to topical composition performance.

The term “fiber powder” refers to any finely divided particulate material, natural or synthetic, formulated for topical application to the scalp or hair for cosmetic coverage, volumizing, or reinforcement purposes.

The term “film former” refers to any polymer, compound, or excipient in a topical composition that creates a cohesive, continuous, and flexible film upon drying or setting, supporting scalp protection, active retention, or improved aesthetics.

The term “follicular microenvironment” refers to the aggregate of biological, chemical, and physical factors surrounding, supporting, or influencing the scalp hair follicles, including matrix components, nutrients, and signaling molecules, which may be modulated by topical treatments.

The term “follicular targeting” refers to any strategy, technique, or property of a topical composition that directs, enhances, or optimizes the delivery and deposition of actives specifically into or around hair follicles, as opposed to surface or epidermal layers.

The term “freeze-dried powder” refers to a composition form where active ingredients, excipients, or nutrients are processed by lyophilization (freeze drying), resulting in a dry, shelf-stable, reconstitutable powder for topical mixing or application.

The term “growth factor” refers to any peptide, protein, or molecule included in a topical composition that stimulates, augments, or supports cell proliferation, differentiation, or regenerative signaling in the scalp or hair follicles, including but not limited to EGF, bFGF, and KGF.

The term “imaging analysis” refers to any method, protocol, or use of imaging technologies (for example, trichoscopy, videodermoscopy, or phototrichogram) to assess, quantify, or monitor scalp, hair density, growth rate, follicle state, or other treatment outcomes.

The term “keratin fiber” refers to natural or synthetic fibers composed primarily of keratin protein incorporated into topical cosmetic compositions as volumizers, concealment agents, or hair structure enhancers.

The term “load gradient” refers to any pattern, design, or distribution of active or additive ingredients across a device, patch, or matrix so that different zones, layers, or regions deliver variable amounts (concentration gradient) of ingredient(s), supporting targeted or sequential release.

The term “locking mechanism” refers to any physical or engineered feature within dispensers, packaging, or devices that controls, restricts, or enables the release, mixing, or administration of a topical composition, preventing unintended activation.

The term “modular dispenser” refers to any dispensing system, container, applicator, or device capable of independently storing, maintaining separation of, mixing, and/or dispensing two or more components, active or inactive ingredients, formulations, or excipients, either simultaneously, sequentially, or per a user-defined, programmable, or predetermined schedule. The modular dispenser enables user or healthcare provider customization of dosing, ingredient ratio, timing, or application protocol, and may include mechanical, digital, electronic, or smart-enabled features that facilitate precision, personalization, or adaptive delivery. This meaning encompasses any design or operational method supporting tailored, combinatorial, or on-demand dispensation within the context of topical drug, cosmetic, or cosmeceutical application.

The term “pH adjuster” refers to any compound, buffer, or ingredient included to set, modify, or maintain the pH of the topical composition at a desired range, thereby optimizing stability, compatibility, or performance of actives.

The term “penetration efficiency” refers to the measure or property of a topical composition, regimen, or enhancer representing the fraction or rate at which active ingredient(s) successfully reach intended scalp, follicular, or biological targets following topical application.

The term “penetration modulator” refers to any additive, excipient, compound, or technology formulated or integrated to alter, increase, or optimize the rate, extent, or pathway by which actives permeate the scalp, epidermis, or follicle, supporting efficacy and safety.

The term “peptide complex” refers to any blend, mixture, or proprietary combination of synthetic or natural peptides incorporated into the topical composition to provide synergistic or additive effects beneficial to hair follicle stimulation, scalp health, or cosmetic outcomes.

The term “point of use” refers to the location, timing, or process in which the final topical composition is mixed, assembled, reconstituted, or administered by the end user, healthcare provider, or caregiver, rather than at central manufacturing.

The term “scalp coverage” refers to any attribute, measurement, or characteristic relating to the extent, area, or completeness of topical composition application or hair fiber cosmetic additive placement on the scalp, relevant to cosmetic and therapeutic outcomes.

The term “scalp health” refers to the sum of biological, physical, and aesthetic parameters relating to scalp tissue integrity, hydration, inflammation status, microbiome balance, and ability to support healthy hair growth, which may be improved by topical treatment.

The term “scalp hydration” refers to any measure, parameter, or condition reflecting the water content, moisture level, or overall hydration status of scalp tissue, which may be modulated by topical compositions, humectants, or hydrogels.

The term “sensory modifier” refers to any compound, excipient, or system incorporated in the topical composition to influence user sensory experience (e.g., fragrance, texturizer, cooling agent) without serving as a principal active.

The term “slow-release system” refers to any technology, vehicle, matrix, or formulation enabling sustained, controlled, or gradual release and absorption of active ingredient(s) over an extended period following administration to the scalp or skin.

The term “strand diameter” refers to the measurement, property, or analytical outcome representing the cross-sectional width of individual hair fibers, typically determined using imaging analysis for efficacy assessment.

The term “tensile strength” refers to any metric, test, or value characterizing the mechanical robustness, elasticity, or force-to-break of hair fibers, relevant to product performance or clinical endpoints, measured using standardized tension testing or analogous protocols.

The term “unit dosage form” refers to any physically discrete, premeasured, or individually packaged quantity of topical composition containing a defined amount of active ingredient(s) intended for administration in a single application.

The term “vitamin/nutraceutical natural product” refers to any vitamin, cofactor, plant-derived, or nutrient-based compound included in the topical composition for cosmetic, supportive, or therapeutic benefit, distinct from primary pharmaceutical actives.

The term “volumizing spray” refers to any sprayable topical product, either cosmetic or cosmeceutical, that contains additives, fibers, polymers, or actives designed to adhere to or expand the volume of the hair shaft, immediately improving appearance or density upon application.

The term “topically administering” (and its grammatical variants) refers to applying a composition directly to a surface of the body, such as the scalp or skin, for localized or regional effect, including via any suitable dosage form such as a solution, lotion, gel, foam, patch, spray, or other vehicle capable of delivering the composition to the desired area.

The term “glass bottle” refers to a container made primarily of glass, configured to store and dispense compositions, and may include any suitable type, grade, shape, color (including clear, amber, or colored), or volume known to those skilled in the art as appropriate for pharmaceutical, cosmetic, or topical formulations.

The term “closure” refers to a device or structure configured to seal, cover, or close the opening of a container (such as a bottle), which may include a cap, lid, stopper, or other securing mechanism, optionally integrated with dispensing features such as a dropper or applicator.

The term “continuous thread finish” refers to a container or closure design that features spiral threads (helical ridges) to enable repeated secure screwing and unscrewing of a cap or closure onto a container neck.

The term “dropper” refers to any device, feature, or assembly configured to enable precise dispensing of liquid, semi-solid, or flowable formulations from a container, including but not limited to pipette-type, bulb-type, or calibrated mechanical dispensers.

The term “UV-protective coating” refers to any material, layer, or treatment that is applied to, integrated within, or forms part of a closure, container, or packaging, and that is designed to reduce, prevent, or delay transmission of ultraviolet (UV) light into the container contents, thereby protecting the formulation or active ingredients from photo-degradation.

The term “scalp” refers to the anatomical region of the head that includes the skin, subcutaneous tissue, and associated hair follicles, and is the surface to which topical compositions are applied for the purposes described.

The term “human afflicted with hair loss, or is at risk thereof” refers to any individual experiencing some degree of reduction in hair density, thickness, or coverage-including but not limited to androgenetic alopecia, alopecia areata, telogen effluvium, or related conditions- or who is prone to, or may be expected to, develop such conditions.

The term “hair density” refers to the number of hair shafts present per unit area of the scalp, and “hair count increase” refers to an increase in the number of visible hair shafts per unit area over a given time period, as may be measured by trichoscopy or other clinical methods.

The term “follicular penetration” (or “follicular deposition”) refers to the process or extent of delivery of an ingredient, active, or composition into, or in close proximity to, hair follicles after topical application.

The term “follicular uptake” refers to the measurable or relative amount of an active or component that is absorbed into hair follicles from a topically applied formulation.

The term “DHT” refers to dihydrotestosterone, an endogenous androgen hormone that affects hair follicle biology and is implicated in androgenetic alopecia and other forms of hair loss.

The terms “administered daily,” “once daily,” and “twice daily” refer to the frequency with which a topical composition is applied to the scalp or other site, meaning one or more times per day, as directed by protocol or user preference.

The term “kit” refers to any collection of two or more components, items, or products provided or packaged together, optionally including a topical composition, container, applicator, instructions for use, or other accessories, designed for coordinated or sequential use as contemplated by the present invention.

The term “pharmaceutical grade” refers to a level of purity, quality, and safety for ingredients or packaging components that is consistent with standards established for pharmaceutical use by recognized regulatory authorities or pharmacopeias.

The term “USP Type III glass” refers to a classification of glass defined by the United States Pharmacopeia as soda-lime glass suitable for storing aqueous, semi-solid, or dry pharmaceutical preparations not intended for parenteral use.

The term “amber glass” refers to glass with an amber or brown color, formulated to absorb wavelengths of light, particularly ultraviolet, to protect contents from photodegradation.

The term “amber glass bottle” refers to a pharmaceutical-grade glass container manufactured in an amber or brown color by incorporation of iron, sulfur, or other colorants, designed to absorb ultraviolet and visible light and reduce photodegradation of enclosed actives or compositions.

The term “photodegradation” refers to the process or outcome by which a chemical compound or active pharmaceutical ingredient is decomposed, altered, or loses potency as a result of exposure to light, particularly ultraviolet or visible wavelengths, thereby reducing shelf life or efficacy of topical compositions.

The term “single-use capsule” refers to any unit-dose packaging form containing an amount of topical composition designed and pre-measured for administration in a single application, which may be in the form of a freeze-dried powder, liquid, or gel and supplied in individual airtight containers for convenient use, stability, and dosing accuracy.

The term “application number” refers to the count or total number of discrete topical doses, aliquots, or administrations deliverable from a given volume or unit of a topical composition or kit, determined by batch analysis or gravimetric dispensing.

The term “freeze-dried powder” refers to a solid-state formulation of active or inactive ingredients produced via lyophilization, in which water is removed under low temperature and pressure to yield a stable, concentrated powder product suitable for reconstitution prior to use.

The term “controlled release” refers to any formulation property, carrier system, or manufacturing technique that enables timed, sustained, or predetermined liberation of active pharmaceutical ingredients from a topical composition after administration, resulting in gradual, prolonged, or pulsed bioavailability at the scalp or hair follicle.

The term “genetic screening kit” refers to any set of materials, devices, reagents, and instructions provided for the purpose of acquiring a biological sample (e.g., buccal swab, saliva, hair) from a subject to determine genetic variants or polymorphisms relevant to hair loss or treatment response, which subsequently inform regimen personalization.

The term “phenotype-based regimen optimization” refers to any protocol, method, or feature within a topical hair regrowth treatment kit or algorithm that adjusts composition, dosing, or scheduling based on the observable physical characteristics, genetic markers, or clinical presentation of an individual.

The term “calibrated,” as used with respect to a dropper or dispensing device, refers to a dropper designed, constructed, and/or marked to consistently deliver a specified and reproducible volume of formulation per actuation.

The term “potency” refers to the proportion or amount of active pharmaceutical ingredient in a composition that remains chemically intact and therapeutically effective after manufacture and storage, relative to the stated or initial concentration.

The term “purified water” refers to water that conforms to relevant pharmacopeial standards for purity and is substantially free from impurities, microorganisms, and contaminants that could affect the stability or safety of topical compositions.

The term “LLLT” refers to low-level light therapy, a method involving the application of coherent or non-coherent light in the visible red or near-infrared spectrum-typically ranging from 600 to 900 nm wavelength-delivered via laser, LED, or similar devices to the scalp or hair-bearing areas for the purpose of promoting hair follicle activity and regrowth. This modality is utilized as part of, or in adjunct with, topical hair regrowth regimens, delivering energy that stimulates biological processes within the scalp to enhance follicular revival, increase hair density, prolong the anagen (growth) phase, and support scalp health. Devices and protocols for LLLT may vary in light source type, energy density, emission pattern, and coverage area, but all are encompassed under the broad definition as systems and methods that use controlled, low-power light exposure to positively affect hair biology and regrowth outcomes when applied to the scalp or affected regions.

### TECHNICAL FIELD

The present invention relates to advanced topical compositions, technologies, and systems for promoting hair regrowth, preventing hair loss, and improving scalp health through a combination of innovative pharmaceutical, device, and packaging strategies. The present invention addresses longstanding limitations in conventional hair regrowth products, focusing on enhanced follicular delivery, improved tolerability, personalized regimen design, and long-term stability. Its comprehensive scope encompasses both the formulation of topical compositions and the integration of delivery devices, diagnostic tools, and user-tailored kits to expand therapeutic efficacy, patient compliance, and product differentiation.

### Topical Compositions and Principal Features

At the heart of the present invention is a family of topical compositions that incorporate at least one active pharmaceutical ingredient chosen from finasteride, minoxidil, dutasteride, and latanoprost, often combined with additional agents such as retinoic acid, hydrocortisone, and aloe vera. These compositions are engineered with advanced penetration enhancers, including, e.g., Transcutol®, laurocapram, dimethyl isosorbide, menthol, and a-bisabolol, formulated to maximize the proportion of actives delivered into hair follicles rather than remaining trapped on the skin's surface. The present invention further specifies precise particle size distributions—such as D90 ≤8.5 μm and optimized Span values—combined with vehicles like propylene glycol, ethanol, and water to support both solubility and targeted delivery. These design choices result in improved follicular penetration and higher local bioavailability, while minimizing systemic exposure and cutaneous irritation.

The compositions are adaptable across a wide range of physical forms, including solutions, gels, foams, hydrogels, bioadhesive matrices, nanoparticles, and phase-change systems. Many embodiments utilize bioadhesive polymers, such as chitosan or hyaluronic acid, to prolong scalp residence time and sustain the release or retention of actives. The present invention further contemplates additional excipients and adjuncts, including antioxidants (e.g., BHT, EDTA), humectants, pH buffers, chelators, antimicrobial agents (e.g., ketoconazole, zinc pyrithione), probiotic/prebiotic additives, as well as botanical and peptide actives that support a holistic approach to scalp and hair health. The technology accommodates hypoallergenic, sulfate- and paraben-free formulations, and features ingredients and antioxidant systems that enable extended chemical stability of actives, preserving product potency over a multi-year shelf life under typical storage conditions.

### Methods of Use and Key Therapeutic Modalities

The present invention provides methods for treating a broad array of hair-related conditions, encompassing androgenetic alopecia, female pattern hair loss, alopecia areata, telogen effluvium, and general scalp health maintenance. The topical compositions are administered directly to the scalp, often in daily or twice-daily regimens, to prevent hair loss, reduce hair shedding, promote the shift from vellus to terminal hair, and increase overall hair density and shaft diameter. Clinical benefit is substantiated by metrics such as hair count, density improvement, and anagen-to-telogen hair ratios, as measured with established trichoscopic and imaging protocols.

The present invention allows for substantial flexibility and personalization in treatment protocols. Methods of use may further include combination approaches, such as co-administration with device-assisted delivery techniques (for example, microneedling, low-level light therapy, ultrasound, or microcurrent stimulation), which serve to amplify follicular uptake and efficacy. In certain embodiments, stepwise protocols including follicle-priming agents (e.g., caffeine or mild acids) or scalp conditioning steps can precede or follow administration of the main actives, further optimizing absorption and patient comfort. Companion app-guided or sensor-driven protocols enable dynamically tailored scheduling and dosing adjustments in response to individual biological feedback or measured scalp parameters, supporting both precision medicine and user compliance. The compositions' engineered tolerability—achieved through anti-inflammatory actives and avoidance of irritants—permits higher or more frequent dosing with significantly reduced risk of irritation compared to traditional alcohol-based solutions.

### Kits, Devices, and Personalization Technologies

A defining innovation of the present invention is the integration of the topical compositions into customizable kits and advanced dispensing and diagnostic systems. The kits may comprise calibrated glass bottles (often amber, with UV-protective coatings to preserve actives), droppers or spray applicators, and multi-chambered or modular bottles that enable separate storage and point-of-use mixing of actives, penetration enhancers, and conditioning adjuncts. This modular approach not only extends product shelf stability but also empowers users or clinicians to individualize ingredient ratios, application steps, and regimen scheduling.

The present invention contemplates the integration of sensor-guided, Bluetooth-enabled, and app-connected devices that track dosing, monitor scalp health (such as pH, hydration, or inflammation), and provide feedback or automated recommendations to optimize therapy. These systems may employ machine learning algorithms to analyze treatment progress and adjust active dosing or scheduling to maximize individual efficacy and minimize side effects. Further, the kits may include auxiliary devices, such as microneedling rollers or low-level light therapy apparatuses, for device-assisted follicular delivery, as well as colorimetric or wearable feedback devices that monitor regimen compliance and adverse reactions.

To address the full patient journey, kit configurations also allow for the inclusion of post-treatment scalp barrier restoration agents, cosmetic hair fibers for immediate visual improvement, and even genetic screening tools for advanced phenotype-based regimen optimization. Freeze-dried or waterless concentrate forms, as well as antimicrobial features, expand usability, portability, and market reach.

### Principal Advantages and Applications

Through the synergistic application of optimized formulations, advanced penetration and delivery strategies, device integration, and user-tailored packaging, the present invention offers notable improvements in efficacy, tolerability, stability, and consumer experience over existing hair regrowth therapies. Core advantages include increased follicular penetration and retention of active agents, enhanced clinical outcomes in hair density and quality, substantially improved formulation stability, reduced local irritation, and expanded flexibility for regimen customization and digital health integration. These attributes render the present invention suitable for a wide spectrum of users, from those with genetic or autoimmune hair loss syndromes to individuals seeking to optimize and personalize their scalp and hair care practices. The present invention thus represents a holistic, technologically advanced platform for next-generation hair regrowth and scalp health management.

### Specific Ranges, Values, and Embodiments

The specific embodiments describing the ranges and values provided below are for illustration purposes only, and do not otherwise limit the scope of the disclosed subject matter, as defined by the claims.

### Active Ingredient

In specific embodiments, the topical composition includes finasteride.

In specific embodiments, the topical composition includes finasteride, 0.3 wt. %.

In specific embodiments, the topical composition includes finasteride, 0.25 wt. %.

In specific embodiments, the topical composition includes finasteride, 0.1 wt. %.

In specific embodiments, the topical composition includes finasteride, 0.1-2.5 wt. %.

In specific embodiments, the topical composition includes finasteride, 2.5 wt. %.

In specific embodiments, the topical composition includes minoxidil.

In specific embodiments, the topical composition includes minoxidil, 8.0 wt. %.

In specific embodiments, the topical composition includes minoxidil, 5 wt. %.

In specific embodiments, the topical composition includes minoxidil, 3.5 wt. %.

In specific embodiments, the topical composition includes minoxidil, 3 wt. %.

In specific embodiments, the topical composition includes minoxidil, 2 wt. %.

In specific embodiments, the topical composition includes dutasteride.

In specific embodiments, the topical composition includes dutasteride, 0.3 wt. %.

In specific embodiments, the topical composition includes dutasteride, 0.1 wt. %.

In specific embodiments, the topical composition includes latanoprost.

In specific embodiments, the topical composition includes latanoprost, 0.005 wt. %.

In specific embodiments, the topical composition includes finasteride and minoxidil.

In specific embodiments, the topical composition includes finasteride, 0.3 wt. %; and minoxidil, 8.0 wt. %.

In specific embodiments, the topical composition includes dutasteride and minoxidil.

In specific embodiments, the topical composition includes dutasteride, 0.3 wt. % and 8.0 wt. % minoxidil.

In specific embodiments, the topical composition includes finasteride, dutasteride, and minoxidil.

In specific embodiments, the topical composition includes finasteride, 0.1 wt. %; dutasteride, 0.1 wt. %; and minoxidil, 8.0 wt. %.

In specific embodiments, the topical composition includes finasteride and minoxidil.

In specific embodiments, the topical composition includes finasteride, 0.25 wt. % and minoxidil, 8.0 wt. %.

In specific embodiments, the topical composition includes latanoprost, finasteride, and minoxidil.

In specific embodiments, the topical composition includes latanoprost, 0.005 wt. %; finasteride, 0.3 wt. %; and minoxidil, 8.0 wt. %.

In specific embodiments, the topical composition includes finasteride and minoxidil.

In specific embodiments, the topical composition includes finasteride, 0.1-2.5%; and minoxidil, 3-5 wt. %.

In specific embodiments, the topical composition includes finasteride, 2.5%; and minoxidil, 3 wt. %.

In specific embodiments, the topical composition includes one or more active ingredients (e.g., finasteride, minoxidil, dutasteride, and/or latanoprost).

### Topical Composition

In specific embodiments, the topical composition includes finasteride (0.3 wt. %), minoxidil (8.0 wt. %), retinoic acid (0.001 wt. %), hydrocortisone (1.0 wt. %), aloe vera, propylene glycol, ethyl alcohol, and penetration enhancer(s).

In specific embodiments, the topical composition includes dutasteride (0.3 wt. %), minoxidil (8.0 wt. %), retinoic acid (0.001 wt. %), hydrocortisone (1.0 wt. %), aloe vera, propylene glycol, ethyl alcohol, and penetration enhancer(s).

In specific embodiments, the topical composition includes finasteride (0.1 wt. %), dutasteride (0.1 wt. %), minoxidil (8.0 wt. %), retinoic acid (0.001 wt. %), hydrocortisone (1.0 wt. %), aloe vera, propylene glycol, ethyl alcohol, and penetration enhancer(s).

In specific embodiments, the topical composition includes minoxidil (8.0 wt. %), retinoic acid (0.001 wt. %), hydrocortisone (1.0 wt. %), aloe vera, propylene glycol, ethyl alcohol, and penetration enhancer(s).

In specific embodiments, the topical composition includes finasteride (0.25 wt. %), minoxidil (8.0 wt. %), retinoic acid (0.001 wt. %), hydrocortisone (1.0 wt. %), aloe vera, propylene glycol, ethyl alcohol, and penetration enhancer(s).

In specific embodiments, the topical composition includes latanoprost (0.005 wt. %), finasteride (0.3 wt. %), minoxidil (8.0 wt. %), retinoic acid (0.001 wt. %), hydrocortisone (1.0 wt. %), aloe vera, propylene glycol, ethyl alcohol, and penetration enhancer(s).

In specific embodiments, the topical composition includes finasteride (0.1-2.5 wt. %), minoxidil (3-5 wt. %), ethanol, propylene glycol, purified water, and penetration enhancer(s).

In specific embodiments, the topical composition includes finasteride (2.5 wt. %), minoxidil (3 wt. %), Siloxyssystem™ gel, ethanol, propylene glycol, purified water, and penetration enhancer(s).

In specific embodiments, the topical composition includes minoxidil (5 wt. %), ethyl alcohol, propylene glycol, purified water, and penetration enhancer(s).

In specific embodiments, the topical composition includes minoxidil (2 wt. % or 5 wt. %), ethyl alcohol, propylene glycol, purified water, and penetration enhancer(s).

In specific embodiments, the topical composition includes the antioxidant system: 0.05% BHT+0.1% EDTA (prevents ≤98% minoxidil degradation at 40° C./75% RH).

In specific embodiments, the ethyl alcohol employed is denatured with 0.5% Bitrex® (avoids misuse).

In specific embodiments, the propylene glycol employed includes <0.1% propionaldehyde (USP <631>).

### Particle Size of Active Ingredient

In specific embodiments, when the active pharmaceutical ingredient is present as a suspended particulate and not fully dissolved in the vehicle, it is formulated as particles having a specified size.

In specific embodiments, when the active pharmaceutical ingredient is present as a suspended particulate and not fully dissolved in the vehicle, it is formulated as particles having D90 ≤8.5 μm, as measured on the reconstituted topical product and not the bulk raw material.

In specific embodiments, when the active pharmaceutical ingredient is present as a suspended particulate and not fully dissolved in the vehicle, it is formulated as particles having a Span value ≤1.7, as measured on the reconstituted topical product and not the bulk raw material.

In specific embodiments, the topical composition includes (i.e., is formulated with) minoxidil particles ≤10 μm, for enhanced follicular penetration.

In specific embodiments, the topical composition includes (i.e., is formulated with) minoxidil particles ≤5 μm, for enhanced follicular penetration.

In specific embodiments, the topical composition includes (i.e., is formulated with) minoxidil particles ≤2.5 μm, for enhanced follicular penetration.

In specific embodiments, the topical composition includes (i.e., is formulated with) minoxidil particles having D₁₀ ≤10 μm, for enhanced follicular penetration.

In specific embodiments, the topical composition includes (i.e., is formulated with) minoxidil particles having D₁₀ ≤5 μm, for enhanced follicular penetration.

In specific embodiments, the topical composition includes (i.e., is formulated with) minoxidil particles having D₁₀ ≤2.5 μm, for enhanced follicular penetration.

In specific embodiments, the topical composition includes (i.e., is formulated with) minoxidil particles having D₅₀ ≤10 μm, for enhanced follicular penetration.

In specific embodiments, the topical composition includes (i.e., is formulated with) minoxidil particles having D₅₀ ≤5 μm, for enhanced follicular penetration.

In specific embodiments, the topical composition includes (i.e., is formulated with) minoxidil particles having D₅₀ ≤2.5 μm, for enhanced follicular penetration.

In specific embodiments, the topical composition includes (i.e., is formulated with) minoxidil particles having D₉₀ ≤10 μm, for enhanced follicular penetration.

In specific embodiments, the topical composition includes (i.e., is formulated with) minoxidil particles having D₉₀ ≤5 μm, for enhanced follicular penetration.

In specific embodiments, the topical composition includes (i.e., is formulated with) minoxidil particles having D₉₀ ≤2.5 μm, for enhanced follicular penetration.

In specific embodiments, the topical composition includes (i.e., is formulated with) minoxidil particles having D₁₀ ≤2.5 μm, D₅₀ ≤5.5 μm, D₉₀ ≤8.5 μm (laser diffraction data).

In specific embodiments, the topical composition includes (i.e., is formulated with) minoxidil particles having D₁₀ ≤2 μm, D₅₀ ≤5 μm, D₉₀ ≤8 μm (laser diffraction data).

In specific embodiments, the topical composition includes (i.e., is formulated with) minoxidil particles having D₁₀ ≤1.5 μm, D₅₀ ≤4.5 μm, D₉₀ ≤7.5 μm (laser diffraction data).

In specific embodiments, the topical composition includes (i.e., is formulated with) finasteride particles ≤10 μm, for enhanced follicular penetration.

In specific embodiments, the topical composition includes (i.e., is formulated with) finasteride particles ≤5 μm, for enhanced follicular penetration.

In specific embodiments, the topical composition includes (i.e., is formulated with) finasteride particles ≤2.5 μm, for enhanced follicular penetration.

In specific embodiments, the topical composition includes (i.e., is formulated with) finasteride particles having D₁₀ ≤10 μm, for enhanced follicular penetration.

In specific embodiments, the topical composition includes (i.e., is formulated with) finasteride particles having D₁₀ ≤5 μm, for enhanced follicular penetration.

In specific embodiments, the topical composition includes (i.e., is formulated with) finasteride particles having D₁₀ ≤2.5 am, for enhanced follicular penetration.

In specific embodiments, the topical composition includes (i.e., is formulated with) finasteride particles having D₅₀ ≤10 μm, for enhanced follicular penetration.

In specific embodiments, the topical composition includes (i.e., is formulated with) finasteride particles having D₅₀ ≤5 μm, for enhanced follicular penetration.

In specific embodiments, the topical composition includes (i.e., is formulated with) finasteride particles having D₅₀ ≤2.5 μm, for enhanced follicular penetration.

In specific embodiments, the topical composition includes (i.e., is formulated with) finasteride particles having D₉₀ ≤10 μm, for enhanced follicular penetration.

In specific embodiments, the topical composition includes (i.e., is formulated with) finasteride particles having D₉₀ ≤5 μm, for enhanced follicular penetration.

In specific embodiments, the topical composition includes (i.e., is formulated with) finasteride particles having D₉₀ ≤2.5 μm, for enhanced follicular penetration.

In specific embodiments, the topical composition includes (i.e., is formulated with) finasteride particles having Span value ≤1.7 (Span=(D₉₀−D₁₀)/D₅₀).

In specific embodiments, the topical composition includes (i.e., is formulated with) finasteride particles having Span value ≤1.6 (Span=(D₉₀−D₁₀)/D₅₀).

In specific embodiments, the topical composition includes (i.e., is formulated with) finasteride particles having Span value ≤1.5 (Span=(D₉₀−D₁₀)/D₅₀).

In specific embodiments, the topical composition includes (i.e., is formulated with) finasteride particles having Span value ≤1.4 (Span=(D₉₀−D₁₀)/D₅₀).

In specific embodiments, the topical composition includes (i.e., is formulated with) finasteride particles having Span value ≤1.3 (Span=(D₉₀−D₁₀)/D₅₀).

In specific embodiments, the topical composition includes (i.e., is formulated with) finasteride particles having Span value ≤1.2 (Span=(D₉₀−D₁₀)/D₅₀).

In specific embodiments, the topical composition includes (i.e., is formulated with) finasteride particles having Span value ≤1.1 (Span=(D₉₀−D₁₀)/D₅₀).

In specific embodiments, the topical composition includes (i.e., is formulated with) dutasteride particles ≤10 μm, for enhanced follicular penetration.

In specific embodiments, the topical composition includes (i.e., is formulated with) dutasteride particles ≤5 μm, for enhanced follicular penetration.

In specific embodiments, the topical composition includes (i.e., is formulated with) dutasteride particles ≤2.5 μm, for enhanced follicular penetration.

In specific embodiments, the topical composition includes (i.e., is formulated with) dutasteride particles having D₁₀ ≤10 μm, for enhanced follicular penetration.

In specific embodiments, the topical composition includes (i.e., is formulated with) dutasteride particles having D₁₀ ≤5 μm, for enhanced follicular penetration.

In specific embodiments, the topical composition includes (i.e., is formulated with) dutasteride particles having D₁₀ ≤2.5 μm, for enhanced follicular penetration.

In specific embodiments, the topical composition includes (i.e., is formulated with) dutasteride particles having D₅₀ ≤10 μm, for enhanced follicular penetration.

In specific embodiments, the topical composition includes (i.e., is formulated with) dutasteride particles having D₅₀ ≤5 μm, for enhanced follicular penetration.

In specific embodiments, the topical composition includes (i.e., is formulated with) dutasteride particles having D₅₀ ≤2.5 μm, for enhanced follicular penetration.

In specific embodiments, the topical composition includes (i.e., is formulated with) dutasteride particles having D₉₀ ≤10 μm, for enhanced follicular penetration.

In specific embodiments, the topical composition includes (i.e., is formulated with) dutasteride particles having D₉₀ ≤5 μm, for enhanced follicular penetration.

In specific embodiments, the topical composition includes (i.e., is formulated with) dutasteride particles having D₉₀ ≤2.5 μm, for enhanced follicular penetration.

### Viscosity

In specific embodiments, the topical composition has a viscosity of 1,250-3,750 cP (Brookfield RV DV-III Ultra, spindle #7, 20 rpm).

In specific embodiments, the topical composition has a viscosity of 1,500-3,500 cP (Brookfield RV DV-III Ultra, spindle #7, 20 rpm).

In specific embodiments, the topical composition has a viscosity of 1,750-3,250 cP (Brookfield RV DV-III Ultra, spindle #7, 20 rpm).

In specific embodiments, the topical composition has a viscosity of 2,500±1,750 cP (Brookfield RV DV-III Ultra, spindle #7, 20 rpm).

In specific embodiments, the topical composition has a viscosity of 2,500±1,500 cP (Brookfield RV DV-III Ultra, spindle #7, 20 rpm).

In specific embodiments, the topical composition has a viscosity of 2,500±1,250 cP (Brookfield RV DV-III Ultra, spindle #7, 20 rpm).

In specific embodiments, the topical composition has a viscosity of 2,500±1,000 cP (Brookfield RV DV-III Ultra, spindle #7, 20 rpm).

In specific embodiments, the topical composition has a viscosity of 2,500±750 cP (Brookfield RV DV-III Ultra, spindle #7, 20 rpm).

In specific embodiments, the topical composition has a viscosity of 2,500±500 cP (Brookfield RV DV-III Ultra, spindle #7, 20 rpm).

In specific embodiments, the topical composition has a viscosity of 2,500±250 cP (Brookfield RV DV-III Ultra, spindle #7, 20 rpm).

### Yield Stress

In specific embodiments, the topical composition has a yield stress of 12.5-30 Pa (controlled squeeze-bottle dispensing).

In specific embodiments, the topical composition has a yield stress of 15-25 Pa (controlled squeeze-bottle dispensing).

In specific embodiments, the topical composition has a yield stress of 17-20 Pa (controlled squeeze-bottle dispensing).

### Thixotropic Index

In specific embodiments, the topical composition has a thixotropic index of ≥1.5 (10 rpm/100 rpm viscosity ratio).

In specific embodiments, the topical composition has a thixotropic index of ≥1.6 (10 rpm/100 rpm viscosity ratio).

In specific embodiments, the topical composition has a thixotropic index of ≥1.7 (10 rpm/100 rpm viscosity ratio).

In specific embodiments, the topical composition has a thixotropic index of ≥1.8 (10 rpm/100 rpm viscosity ratio).

In specific embodiments, the topical composition has a thixotropic index of ≥1.9 (10 rpm/100 rpm viscosity ratio).

In specific embodiments, the topical composition has a thixotropic index of ≥2.0 (10 rpm/100 rpm viscosity ratio).

pH

In specific embodiments, the topical composition has a pH range: 5.7-6.4 (optimized for chemical stability+follicular targeting).

In specific embodiments, the topical composition has a pH range: 5.8-6.3 (optimized for chemical stability+follicular targeting).

In specific embodiments, the topical composition has a pH range: 5.9-6.2 (optimized for chemical stability+follicular targeting).

In specific embodiments, the topical composition has a pH of 5.7±0.1.

In specific embodiments, the topical composition has a pH of 5.8±0.1.

In specific embodiments, the topical composition has a pH of 5.9±0.1.

In specific embodiments, the topical composition has a pH of 6.0±0.1.

In specific embodiments, the topical composition has a pH of 6.1±0.1.

In specific embodiments, the topical composition has a pH of 6.2±0.1.

In specific embodiments, the topical composition has a pH of 6.3±0.1.

### Drying Time

In specific embodiments, the topical composition rapidly dries upon topical administration: <60 sec drying time (10 μL/cm² application).

In specific embodiments, the topical composition rapidly dries upon topical administration: <45 sec drying time (10 μL/cm² application).

In specific embodiments, the topical composition rapidly dries upon topical administration: <30 sec drying time (10 μL/cm² application).

In specific embodiments, the topical composition rapidly dries upon topical administration: <25 sec drying time (10 μL/cm² application).

In specific embodiments, the topical composition rapidly dries upon topical administration: <20 sec drying time (10 μL/cm² application).

### Batch Uniformity

In specific embodiments, the topical composition is manufactured with batch-to-batch consistency Relative Standard Deviation (RSD) <10%.

In specific embodiments, the topical composition is manufactured with batch-to-batch consistency Relative Standard Deviation (RSD) <9%.

In specific embodiments, the topical composition is manufactured with batch-to-batch consistency Relative Standard Deviation (RSD) <8%.

In specific embodiments, the topical composition is manufactured with batch-to-batch consistency Relative Standard Deviation (RSD) <7%.

In specific embodiments, the topical composition is manufactured with batch-to-batch consistency Relative Standard Deviation (RSD) <6%.

In specific embodiments, the topical composition is manufactured with batch-to-batch consistency Relative Standard Deviation (RSD) <5%.

In specific embodiments, the topical composition is manufactured with batch-to-batch consistency Relative Standard Deviation (RSD) <4%.

In specific embodiments, the topical composition is manufactured with batch-to-batch consistency Relative Standard Deviation (RSD) <3%.

In specific embodiments, the topical composition is manufactured with batch-to-batch consistency Relative Standard Deviation (RSD) <2%.

### Penetration Enhancer

In specific embodiments, the topical composition includes one or more penetration enhancers (e.g., Transcutol® (diethylene glycol monoethyl ether), cetyl alcohol, oleic acid, eucalyptol, polyethylene glycol, octyl salicylate, SiloxysSystem™ gel, laurocapram, dimethyl isosorbide (DMI), urea (carbamide), d-limonene, menthol, α-bisabolol, and/or Azone® (laurocapram)).

In specific embodiments, the topical composition includes Transcutol® (diethylene glycol monoethyl ether).

In specific embodiments, the topical composition includes cetyl alcohol.

In specific embodiments, the topical composition includes oleic acid.

In specific embodiments, the topical composition includes eucalyptol.

In specific embodiments, the topical composition includes polyethylene glycol.

In specific embodiments, the topical composition includes octyl salicylate.

In specific embodiments, the topical composition includes SiloxysSystem™ gel.

In specific embodiments, the topical composition includes laurocapram.

In specific embodiments, the topical composition includes dimethyl isosorbide (DMI).

In specific embodiments, the topical composition includes urea (carbamide).

In specific embodiments, the topical composition includes d-limonene.

In specific embodiments, the topical composition includes menthol.

In specific embodiments, the topical composition includes α-bisabolol.

In specific embodiments, the topical composition includes Azone® (laurocapram).

In specific embodiments, the topical composition includes one or more penetration enhancers that increase follicular deposition of the active ingredient(s) by ≥40% compared to formulations without said enhancer, as measured by in vitro Franz cell diffusion assay using porcine ear skin.

In specific embodiments, the topical composition includes one or more penetration enhancers that increase follicular deposition of the active ingredient(s) by ≥50% compared to formulations without said enhancer, as measured by in vitro Franz cell diffusion assay using porcine ear skin.

In specific embodiments, the topical composition includes one or more penetration enhancers that increase follicular deposition of the active ingredient(s) by ≥60% compared to formulations without said enhancer, as measured by in vitro Franz cell diffusion assay using porcine ear skin.

In specific embodiments, the topical composition includes one or more penetration enhancers that reduce epidermal retention time of the active ingredient(s) to ≤15 minutes while maintaining ≥60% follicular penetration efficiency.

In specific embodiments, the topical composition includes one or more penetration enhancers that reduce epidermal retention time of the active ingredient(s) to ≤15 minutes while maintaining ≥70% follicular penetration efficiency.

In specific embodiments, the topical composition includes one or more penetration enhancers that reduce epidermal retention time of the active ingredient(s) to ≤15 minutes while maintaining ≥80% follicular penetration efficiency.

In specific embodiments, the topical composition includes one or more penetration enhancers that reduce epidermal retention time of the active ingredient(s) to ≤15 minutes while maintaining ≥90% follicular penetration efficiency.

In specific embodiments, the topical composition includes one or more penetration enhancers that achieve stratum corneum disruption quantified by ≥30% reduction in transepidermal water loss (TEWL) within 30 minutes post-application.

In specific embodiments, the topical composition includes one or more penetration enhancers that achieve stratum corneum disruption quantified by ≥35% reduction in transepidermal water loss (TEWL) within 30 minutes post-application.

In specific embodiments, the topical composition includes one or more penetration enhancers that achieve stratum corneum disruption quantified by ≥40% reduction in transepidermal water loss (TEWL) within 30 minutes post-application.

### Methods of Medical Treatment

In specific embodiments, the method of medical treatment described herein includes at least one of: a method of treating alopecia, method of promoting hair growth, method of preventing hair loss, method of reducing hair shedding, method of increasing hair density or thickness, method of treating androgenetic alopecia (male pattern baldness), method of treating alopecia areata or autoimmune-related hair loss, method of inducing hair regeneration or follicle neogenesis, method of treating female pattern hair loss (FPHL), method of treating female scalp hair loss, and method of treating female pattern alopecia.

In specific embodiments, the method of medical treatment described herein increases hair density by ≥20% in androgenetic alopecia patients, as measured by trichoscopy at 24 weeks.

In specific embodiments, the method of medical treatment described herein increases hair density by ≥25% in androgenetic alopecia patients, as measured by trichoscopy at 24 weeks.

In specific embodiments, the method of medical treatment described herein increases hair density by ≥30% in androgenetic alopecia patients, as measured by trichoscopy at 24 weeks.

In specific embodiments, the method of medical treatment described herein increases hair density by ≥35% in androgenetic alopecia patients, as measured by trichoscopy at 24 weeks.

In specific embodiments, the topical composition is administered daily.

In specific embodiments, the topical composition is administered once daily.

In specific embodiments, the topical composition is administered twice daily.

In specific embodiments, the topical composition is administered daily with low-level light therapy (630-670 nm), 1-7 times per week.

In specific embodiments, the topical composition is administered daily with daily microneedling (0.3 mm).

In specific embodiments, the topical composition is administered daily with microneedling (0.3 mm) 1-7 times a week.

In specific embodiments, the topical composition is administered daily with weekly microneedling (0.3 mm).

In specific embodiments, the topical composition is administered daily with daily oral administration of finasteride.

In specific embodiments, the topical composition is administered daily with daily oral administration of minoxidil.

In specific embodiments, the topical composition is administered daily with daily oral administration of dutasteride.

In specific embodiments, the topical composition is applied to the scalp of a human with WNT10Agene polymorphism.

In specific embodiments, the topical composition is applied to the scalp of a male.

In specific embodiments, the topical composition is applied to the scalp of a male with male pattern baldness.

In specific embodiments, the topical composition is applied to the scalp of a female.

In specific embodiments, the topical composition is applied to the scalp of a postmenopausal female with Ludwig stage II-III FPHL.

In specific embodiments, the topical composition is applied to the scalp of a human, and the composition exhibits ≥5% reduction in hair loss vs. no treatment.

In specific embodiments, the topical composition is applied to the scalp of a human, and the composition exhibits ≥10% reduction in hair loss vs. no treatment.

In specific embodiments, the topical composition is applied to the scalp of a human, and the composition exhibits ≥15% reduction in hair loss vs. no treatment.

In specific embodiments, the topical composition is applied to the scalp of a human, and the composition exhibits ≥20% reduction in hair loss vs. no treatment.

In specific embodiments, the topical composition is applied to the scalp of a human, and the composition exhibits ≥25% reduction in hair loss vs. no treatment.

In specific embodiments, the topical composition is applied to the scalp of a human, and the composition reduces scalp DHT by ≥50% while increasing follicular uptake 3-fold.

In specific embodiments, the topical composition is applied to the scalp of a human, and the composition reduces scalp DHT by ≥60% while increasing follicular uptake 3-fold.

In specific embodiments, the topical composition is applied to the scalp of a human, and the composition reduces scalp DHT by ≥70% while increasing follicular uptake 3-fold.

In specific embodiments, the topical composition is applied to the scalp of a human, and the composition enhances follicular penetration with minimal or no irritation.

In specific embodiments, the topical composition is applied to the scalp of a human, and within 6 months the hair count increase ≥20 hairs/cm².

In specific embodiments, the topical composition is applied to the scalp of a human, and within 6 months the hair count increase ≥25 hairs/cm².

In specific embodiments, the topical composition is applied to the scalp of a human, and within 6 months the hair count increase ≥30 hairs/cm².

In specific embodiments, the topical composition is applied to the scalp of a human, and within 6 months the hair count increase ≥35 hairs/cm².

In specific embodiments, the topical composition is applied to the scalp of a human, and within 6 months the hair count increase ≥40 hairs/cm².

In specific embodiments, the topical composition is applied to the scalp of a human, and within 6 months the hair count increase ≥45 hairs/cm².

In specific embodiments, the topical composition is applied to the scalp of a human, and within 6 months the hair count increase ≥42.5 hairs/cm².

In specific embodiments, the topical composition is applied to the scalp of a human, and within 6 months the hair count increase ≥47.5 hairs/cm².

In specific embodiments, the topical composition is applied to the scalp of a human, and within 6 months the hair count increase ≥50 hairs/cm².

In specific embodiments, the topical composition is applied to the scalp of a human, and within 6 months the hair count increase ≥52.5 hairs/cm².

In specific embodiments, the topical composition is applied to the scalp of a human, and within 6 months the hair count increase ≥55 hairs/cm².

In specific embodiments, the topical composition is applied to the scalp of a human, and within 12 weeks the hair density increases ≥20 hairs/cm².

In specific embodiments, the topical composition is applied to the scalp of a human, and within 12 weeks the hair density increases ≥22.5 hairs/cm².

In specific embodiments, the topical composition is applied to the scalp of a human, and within 12 weeks the hair density increases ≥25 hairs/cm².

In specific embodiments, the topical composition is applied to the scalp of a human, and within 24 weeks the hair density increases ≥40 hairs/cm².

In specific embodiments, the topical composition is applied to the scalp of a human, and within 24 weeks the hair density increases ≥45 hairs/cm².

In specific embodiments, the topical composition is applied to the scalp of a human, and within 24 weeks the hair density increases ≥50 hairs/cm².

In specific embodiments, the topical composition is applied to the scalp of a human, and within 24 weeks the hair density increases ≥52.5 hairs/cm².

In specific embodiments, the topical composition is applied to the scalp of a human, and within 24 weeks the hair density increases ≥55 hairs/cm².

In specific embodiments, the topical composition is applied to the scalp of a human, and within 12 weeks the hair shaft diameter increases ≥0.002 mm.

In specific embodiments, the topical composition is applied to the scalp of a human, and within 12 weeks the hair shaft diameter increases ≥0.003 mm.

In specific embodiments, the topical composition is applied to the scalp of a human, and within 12 weeks the hair shaft diameter increases ≥0.004 mm.

In specific embodiments, the topical composition is applied to the scalp of a human, and within 24 weeks the hair shaft diameter increases ≥0.010 mm.

In specific embodiments, the topical composition is applied to the scalp of a human, and within 24 weeks the hair shaft diameter increases ≥0.013 mm.

In specific embodiments, the topical composition is applied to the scalp of a human, and within 24 weeks the hair shaft diameter increases ≥0.015 mm.

### Stability

In specific embodiments, the topical composition retains ≥50% potency after 12 months at 25° C./60% RH.

In specific embodiments, the topical composition retains ≥60% potency after 12 months at 25° C./60% RH.

In specific embodiments, the topical composition retains ≥70% potency after 12 months at 25° C./60% RH.

In specific embodiments, the topical composition retains ≥80% potency after 12 months at 25° C./60% RH.

In specific embodiments, the topical composition retains ≥90% potency after 12 months at 25° C./60% RH.

In specific embodiments, the topical composition retains ≥95% potency after 12 months at 25° C./60% RH.

In specific embodiments, the topical composition maintains chemical stability for ≥24 months at 2-8° C. (EMA ICH Q5C).

### Nanoparticle or Liposome Delivery

In specific embodiments, the formulation employs nanoparticles, liposomes, and/or solid lipid nanoparticles for improved API penetration to hair follicles. Nanocarriers facilitate enhanced delivery, stabilization, and extended release, offering superior efficacy and patent differentiation beyond bulk solutions.

In specific embodiments, the topical composition includes API-loaded nanoparticles.

In specific embodiments, liposomes encapsulate finasteride for follicular delivery.

In specific embodiments, solid lipid nanoparticles (50-300 nm) stabilize minoxidil and increase follicular uptake by ≥50%, as measured in Franz cell assays.

### Thermoresponsive or Phase-Change Gel System

In specific embodiments, the formulation includes a gel or patch that changes viscosity or phase upon skin contact or mild heating, creating on-demand enhanced API release/permeation.

In specific embodiments, the topical composition is a phase-change gel activated by scalp temperature (>32° C.).

In specific embodiments, administration involves thermoresponsive hydrogel applying API with increased follicular permeability above 30° C.

In specific embodiments, the gel system transitions from liquid to semi-solid within <45 seconds of application, resulting in ≥25% increased retention.

pH-Modulated Release Formulations

In specific embodiments, with the formulations described herein, the API solubility or release is triggered or enhanced by a controlled pH environment, either at the time of administration or during transition through the skin layers. This enables optimized follicular targeting, improved chemical stability, and personalization.

In specific embodiments, the topical composition includes a pH-modifying buffer system, adjusting formulation to pH 5.8-6.3 upon administration.

In specific embodiments, the API is inactive at formulation pH and activated in follicular environment (pH 5-6).

In specific embodiments, user-adjustable pH effectors allow modulation between pH 5.7-6.4 for personalized treatment efficacy.

### Multi-Chambered or Sequential Dispensing Packaging

In specific embodiments, the kit described herein can include multi-compartment bottles/packages enabling sequential or zoned application of differing formulations, e.g., for different scalp regions or stepwise treatment; addresses variable patterns of hair loss.

In specific embodiments, the kit comprises a dual-chamber bottle dispensing finasteride and minoxidil separately.

In specific embodiments, a multi-dose applicator delivers API plus scalp conditioning agent in separate pulses.

In specific embodiments, sequential dispensing packaging allows time-staggered release of follicle-priming and regrowth agents.

### Bioadhesive Matrix System

In specific embodiments, the formulation can include a bioadhesive polymer system for increased residence time on scalp, enabling enhanced API penetration, reduced runoff, and improved patient compliance.

In specific embodiments, the formulation includes a bioadhesive polymer system comprising at least one of chitosan, hyaluronic acid, polyacrylates, polyvinylpyrrolidone (PVP), carbomer, cellulose derivatives (e.g., hydroxyethylcellulose), gelatin, and alginate.

In specific embodiments, the topical composition includes a bioadhesive polysaccharide (e.g., chitosan, hyaluronic acid) for prolonged scalp retention.

In specific embodiments, API is embedded in a bioadhesive hydrogel delivering >60% of dose to follicle within 20 minutes.

In specific embodiments, the bioadhesive matrix contains polymers conferring 30% greater residence time compared to non-bioadhesive formulas.

### Exosome or Stem Cell Conditioned Media Addition

In specific embodiments, the formulation can include exosomes or stem cell-derived conditioned media supporting follicle regeneration, enabling a combination therapy with enhanced regenerative effect.

In specific embodiments, the topical composition includes 1-5% exosome suspension from human adipose-derived stem cells.

In specific embodiments, stem cell conditioned media are blended with minoxidil and penetration enhancer.

In specific embodiments, exosomes constituting 0.05-2% w/w are integrated into the matrix, demonstrated to increase anagen phase follicles.

### Antibacterial/Antifungal Secondary Active System

In specific embodiments, the formulation can additionally include actives (e.g., zinc pyrithione, ketoconazole) for scalp microbiome control, addressing dandruff or fungal overgrowth.

In specific embodiments, the topical composition includes 1-2% ketoconazole for antifungal action along with hair regrowth APIs.

In specific embodiments, zinc pyrithione is present at up to 2% for anti-dandruff performance.

In specific embodiments, betaine or polyhexamethylene biguanide (PHMB) provides antibacterial action at 0.1-1% w/w.

### Probiotic or Prebiotic Scalp Additives

In specific embodiments, the formulation includes probiotic strains or prebiotic substrates to modulate scalp microbiome, reduce inflammation, and enhance API efficacy; which meets consumer demand for holistic scalp health.

In specific embodiments, the topical composition incorporates Lactobacillus ferment lysate at 0.5-5%.

In specific embodiments, a prebiotic oligosaccharide blend (e.g., inulin, fructooligosaccharides) at 0.2-2% is added to promote healthy microbiome.

In specific embodiments, probiotic blends are supplied in freeze-dried capsules reconstituted with formulation at point of use.

### Time-Release Microencapsulation of APIs

In specific embodiments, the API is microencapsulated with biodegradable polymers, providing controlled and sustained release over hours to days; improves dosing convenience and efficacy.

In specific embodiments, minoxidil and finasteride are microencapsulated in poly(lactic-co-glycolic acid) (PLGA) microspheres.

In specific embodiments, controlled-release microspheres (10-100 μm) release API over 4-24 hours post-application.

In specific embodiments, microencapsulated dutasteride achieves ≥30% greater follicular concentration at 8 h than non-encapsulated forms.

### Personalized Regimen Scheduling

In specific embodiments, the kit includes a digital device, wearable, or app that optimizes dosing schedules based on patient feedback, hair density tracking, or response; supports adherence and efficacy.

In specific embodiments, the kit comprises a companion app tracking regimen and prompting dosing.

In specific embodiments, a Bluetooth-enabled dispenser logs application data for physician review.

In specific embodiments, machine learning algorithms recommend regimen changes based on user trichogram results.

### Heat-Activated or Self-Warming Formulation

In specific embodiments, the formulation incorporates mild exothermic agents that warm the formulation upon contact, increasing micropore opening and follicular penetration.

In specific embodiments, the topical composition contains calcium chloride and water-in-oil emulsion producing mild exotherm (up to 40° C.) for 1-3 minutes.

In specific embodiments, self-heating packs integrated with dispenser activate upon mixing and application.

In specific embodiments, heat-activated penetration enhancers boost follicular API uptake by ≥25% compared to ambient formulations.

### Follicle-Priming Step (Stepwise Protocol)

In specific embodiments, the kit includes follicle-priming agents (e.g., caffeine, low-dose retinoids, mild acids) administered before main API regimen; supports more individualized protocols and improved outcomes.

In specific embodiments, the regimen includes a priming composition with 2% caffeine applied 10 minutes before API application.

In specific embodiments, the kit provides separate vials for priming and regrowth phases.

In specific embodiments, priming step includes low-dose glycolic acid (0.05-0.2%) for enhanced follicle opening.

### Low Sensitization or Hypoallergenic Formulation

In specific embodiments, the composition is free of “top 8” allergens, irritants, and is dermatologically tested for sensitive skin; expands applicability and safety profile.

In specific embodiments, the topical composition is formulated without sulfates, parabens, and formaldehyde donors.

In specific embodiments, all excipients are hypoallergenic and have irritation scores ≤1.0/4 in clinical patch testing.

In specific embodiments, allergen-free status is maintained through use of validated ingredient supply chain.

### Aroma-Functional Compliance Boosters

In specific embodiments, the topical composition includes one or more bioactive aroma compounds (e.g., mint, citrus terpenes, sandalwood) chosen to activate olfactory receptors linked to follicle health, mood, and/or compliance.

In specific embodiments, the topical composition includes SANDALORE™ at 0.1-1% as a compliance and mood enhancer.

In specific embodiments, menthol (0.1-0.5%) is added for sensory cooling and improved acceptability.

In specific embodiments, natural fragrance blends with proven trichological benefits are added at 0.01-1%.

### UV-Protectant or Environmental Shielding Agent

In specific embodiments, the formulation integrates one or more photoprotective agents (e.g., zinc oxide, titanium dioxide, ascorbic acid) for mitigation of photo-induced hair loss and active stability.

In specific embodiments, the topical composition contains zinc oxide nanoparticles at 0.1-3% for UV shielding.

In specific embodiments, ascorbic acid, tocotrienols, and polyphenols at 1-10% provide antioxidant protection.

In specific embodiments, a topical environmental shield combines UV filter, antioxidant, and anti-pollution actives in a one-step regimen.

### Waterless or “Concentrate” Formulation

In specific embodiments, the composition is a portable, anhydrous, and/or reconstitutable high-potency product for travel, convenience, and/or reduced preservative/additive requirement.

In specific embodiments, the topical composition is a waterless concentrate reconstituted with purified water prior to use.

In specific embodiments, freeze-dried powder format is supplied in single-use capsules for home mixing.

In specific embodiments, anhydrous gel with API and penetration enhancer delivers equivalent efficacy in ⅓ the mass.

### Energy-Activated (Light/Electric) Release System

In specific embodiments, the formulation or API is released/activated by application of external energy (e.g., laser, LED, mild microcurrent); enables on-demand control and combination device features.

In specific embodiments, the topical composition includes a photoactivatable compound releasing API upon exposure to 650-700 nm light.

In specific embodiments, device-assisted microcurrent (0.5-2.0 mA) increases permeability of API for 30 minutes post-application.

In specific embodiments, the formulation is administered with a scalp comb generating mild field effect to trigger release.

### Multi-API Dose Personalization

In specific embodiments, the packaging or device allows user customization of API dose ratios (e.g., minoxidil/finasteride) at the point of application; meets demands for tailored regimens.

In specific embodiments, a digital dispenser allows minoxidil/finasteride/dutasteride ratios to be set by the user.

In specific embodiments, kit provides three API chambers with calibrated mixing defaults (e.g., 2:1, 1:1) selectable at time of use.

In specific embodiments, auto-blending technology can be adjusted according to response feedback.

### Inclusion of Polymorphic or Genetic Screening Kit

In specific embodiments, the kit is expanded to contain genetic screening tools (e.g., buccal swab DNA test for WNT10A/AR/SRD5A2 variants), enabling regimen personalization and precision medicine claims.

In specific embodiments, the kit includes a buccal swab and instructions for in-home genetic assay prior to regimen start.

In specific embodiments, genetic result-based recommendations are provided via app after lab analysis.

In specific embodiments, claims comprise compositions optimized for use in patients with specific gene polymorphisms.

### Hair Fiber Cosmetic Additive

In specific embodiments, the topical composition includes color-matched hair fibers, keratin powders, and/or temporary volumizers, optionally charged or formulated to enhance adherence, offering immediate cosmetic improvement with regrowth therapy.

In specific embodiments, the kit contains a bonded hair fiber powder matching human hair colors in 10 shades.

In specific embodiments, electrostatically charged keratin fibers are blended with regrowth formulation for scalp application.

In specific embodiments, temporary volumizing spray with embedded growth factors is packaged alongside the composition.

### Mechanical or Device-Assisted Regimen Option

In specific embodiments, the formulation is supplied with companion mechanical device (e.g., microneedling roller, ultrasound patch, microjet sprayer) for improved penetration and proprietary device-method claims.

In specific embodiments, kit includes a 0.3 mm microneedling roller for pre/post application of the formulation.

In specific embodiments, microjet spray device administers liquid API at uniform particle size <10 μm.

In specific embodiments, device pulses (ultrasound, microcurrent) are used synchronously with topical dosing.

### Sensor-Responsive Release Technology

In specific embodiments, the formulation is built to respond to skin surface sensors (e.g., pH, hydration, temperature), unlocking API delivery only when scalp parameters signal need; enables smarter/more adaptive products.

In specific embodiments, the topical composition delivers API only above a scalp hydration threshold, detected by sensor.

In specific embodiments, a patch with integrated sensor triggers active release in response to scalp heat or irritation signals.

In specific embodiments, real-time feedback system halts API release in the event of detected adverse reaction.

### Real-Time Manufacturing Quality Monitoring

In specific embodiments, the process control features in manufacturing (e.g., real-time PSD measurement, viscosity sensors) are integrated, not only for product consistency but as an IP-protected method for superior batch uniformity.

In specific embodiments, manufacturing includes in-line laser diffraction sensors monitoring D10/D50/D90 of API throughout batch.

In specific embodiments, viscosity of the final blend is monitored by real-time rheometry with automatic batch adjustments.

In specific embodiments, content uniformity of APIs is validated through automated HPLC inline analysis before final packaging.

### Scalp Barrier Restoration Agent

In specific embodiments, the topical composition includes one or more additional actives (e.g., ceramides, cholesterol, fatty acids, panthenol) specifically targeting stratum corneum repair post-treatment, enhancing long-term tolerability and differentiating products with scalp health focus.

In specific embodiments, the topical composition further contains 0.01-0.2% ceramide NP for barrier restoration.

In specific embodiments, a separate post-treatment lotion with cholesterol and panthenol is supplied with the kit.

In specific embodiments, barrier restoration agent is embedded in the same matrix, released >60 min post-API administration.

### Smart-Responsive Polymer Carrier System

In specific embodiments, the formulation integrates smart-responsive polymers (e.g., stimuli-sensitive hydrogels) that alter API release rate based on environmental cues such as humidity, sweat, or sebum. This enables dynamic, “on-demand” drug delivery, providing targeted, personalized therapy. Such materials align drug release with scalp physiology, boosting efficacy and minimizing systemic absorption.

In specific embodiments, the topical composition includes a humidity-sensitive hydrogel matrix that modulates API release based on scalp moisture.

In specific embodiments, the carrier polymer network contracts or expands in response to sweat, enhancing penetration when scalp conditions are most conducive.

In specific embodiments, a temperature-responsive polymer causes pulsatile release of finasteride specifically above 32° C., corresponding to post-exercise or summer conditions.

### User-Tailored Modular Delivery Kits

In specific embodiments, the customizable delivery kit allows selection or mixing of actives, penetration enhancers, or conditioning additives at home, according to user needs (e.g., sensitivity, hair type, or treatment zone). This creates a highly personalized regimen with improved satisfaction and compliance, and enables multiple distinct commercial SKUs from a core formulation. The approach integrates trends from companion diagnostics and at-home personalization.

In specific embodiments, the kit provides separate ampoules of actives (finasteride, minoxidil, botanical extracts) to be mixed at point of use.

In specific embodiments, modular dispensers allow user selection of penetration enhancer strength (e.g., “mild/moderate/maximal”).

In specific embodiments, a multi-cartridge applicator dispenses custom ratios of growth factors, antioxidants, and APIs, pre-programmed based on digital guidance.

### Transdermal Microneedle Array Patch System

In specific embodiments, the formulation is delivered via a patch or pad containing a removable or dissolvable microneedle array preloaded with active(s). This system allows painless, controlled, and direct API delivery into the follicular vicinity, minimizing surface losses and ensuring uniform regional dosing. This feature integrates hair regrowth APIs with next-generation medical microneedle platforms.

In specific embodiments, the kit includes a dissolvable microneedle patch loaded with minoxidil and a penetration enhancer.

In specific embodiments, the patch has arrays tuned for temporal (overnight) release and is replaced daily or weekly.

In specific embodiments, APIs are loaded at gradients such that the deepest microneedles deliver a higher fraction of finasteride relative to surface layers.

### Scalp Diagnostic Feedback System

In specific embodiments, employed is an integration of a scalp health sensor or diagnostic device (e.g., measuring moisture, pH, sebum, inflammation) with the regimen, so treatment timing, selection, or intensity responds to real-time user data. This supports precision medicine and feedback-controlled therapy for superior outcomes and supports new digital health business models.

In specific embodiments, the kit comprises an integrated wearable or handheld device monitoring scalp pH and recommending regimen adjustments.

In specific embodiments, a colorimetric strip or digital sensor on the applicator handle changes color if scalp inflammation is detected, prompting adjunct therapy use.

In specific embodiments, the system features Bluetooth connectivity for transmitting ongoing scalp health metrics to a paired treatment optimization app.

### Regenerated Bioactive Scaffold for Follicle Support

In specific embodiments, a biodegradable, topical scaffold formed in situ after application delivers active and physically supports miniaturized follicles during regrowth phases. The scaffold, loaded with APIs, growth factors, or stem cell-derived signals, gradually dissolves, providing both sustained delivery and a microenvironment for follicle recovery. This mechanical-biological hybrid enables patent differentiation for both functional and cosmeceutical/medical markets.

In specific embodiments, the topical composition forms a cross-linked, bioresorbable film following application, spatially localizing APIs to follicular units.

In specific embodiments, the scaffold material contains peptide-based signals or extracellular matrix mimics in combination with active drugs.

In specific embodiments, the scaffold's degradation rate is tuned (24-72 hours) to synchronize with hair cycle transitions or wound-healing after microneedling.

### Advanced Penetration and Targeted Delivery Systems

### Real-Time Particle Size and Viscosity Monitoring in Manufacturing

Specific embodiments of the present invention ensure batch-to-batch consistency, regulatory compliance, and competitive differentiation by delivering reliably uniform products. In-line sensors for real-time laser diffraction (PSD) and rheometry allow immediate corrective adjustments, minimizing risk of out-of-spec batches. Existing technologies report post-hoc analysis; whereas specific embodiments of the present invention provide for dynamic, in-line feedback during production.

In specific embodiments, in-line sensors monitor and adjust API particle size and product viscosity during manufacture.

In specific embodiments, laser diffraction sensors and rheometers are integrated into production lines for continuous feedback.

In specific embodiments, final API D90 ≤8.5 μm, viscosity 2,500±250 cP, and RSD <4% are validated prior to packaging using real-time sensors.

### Follicular Targeted Microencapsulation and Phase-Change Release

Specific embodiments of the present invention enable extended release for sustained efficacy, reduction in dosing frequency, and patent exclusivity over simple solutions. Microencapsulation in PLGA or lipid matrices with phase-change gels prompts “on-demand” release at scalp/body temperature. This is uncommon in standard minoxidil/finasteride topical products, especially with follicular-targeting biopolymers.

In specific embodiments, active APIs are microencapsulated for slow or triggered release at the follicle.

In specific embodiments, phase-change gels transition at scalp temperature (>32° C.) to increase local API concentration.

In specific embodiments, PLGA microspheres (10-100 μm) loaded with minoxidil and finasteride release >50% of dose over 24 hours when exposed to 37° C.

### Bioadhesive Matrix for Prolonged Scalp Retention

Specific embodiments of the present invention minimize runoff, increase efficacy, and enable reduced dosing and improved consumer acceptability vs. conventional formulas. Chitosan/hyaluronic acid or similar bioadhesive polymers increase residence time and deliver APIs much closer to the follicle. Existing topical formulas rarely utilize bioadhesive carriers for follicle-directed delivery.

In specific embodiments, the topical API is embedded in a bioadhesive hydrogel or matrix for increased scalp retention.

In specific embodiments, the topical API is embedded in a bioadhesive polymer system, for increased scalp retention.

In specific embodiments, the topical composition includes a bioadhesive polymer system in up to 5 wt. %.

In specific embodiments, the topical composition includes a bioadhesive polymer system in up to 4.5 wt. %.

In specific embodiments, the topical composition includes a bioadhesive polymer system in up to 4 wt. %.

In specific embodiments, the topical composition includes a bioadhesive polymer system in up to 3.5 wt. %.

In specific embodiments, the topical composition includes a bioadhesive polymer system in up to 2.5 wt. %.

In specific embodiments, the topical composition includes a bioadhesive polymer system in up to 2 wt. %.

In specific embodiments, the topical composition includes a bioadhesive polymer system in at least 0.1 wt. %.

In specific embodiments, the topical composition includes a bioadhesive polymer system in at least 0.2 wt. %.

In specific embodiments, the topical composition includes a bioadhesive polymer system in at least 0.25 wt. %.

In specific embodiments, the topical composition includes a bioadhesive polymer system in at least 0.5 wt. %.

In specific embodiments, the topical composition includes a bioadhesive polymer system in at least 1 wt. %.

In specific embodiments, the topical composition includes a bioadhesive polymer system in 0.1-5 wt. %.

In specific embodiments, the topical composition includes a bioadhesive polymer system in 0.2-5 wt. %.

In specific embodiments, the topical composition includes a bioadhesive polymer system in 2.75±2.7 wt. %.

In specific embodiments, the topical composition includes a bioadhesive polymer system in 2.75±2.5 wt. %.

In specific embodiments, the topical composition includes a bioadhesive polymer system in 2.75±2.25 wt. %.

In specific embodiments, the topical composition includes a bioadhesive polymer system in 2.75±2 wt. %.

In specific embodiments, the topical composition includes a bioadhesive polymer system in 2.75±1.75 wt. %.

In specific embodiments, the topical composition includes a bioadhesive polymer system in 2.75±1.5 wt. %.

In specific embodiments, the topical composition includes a bioadhesive polymer system in 2.75±1.25 wt. %.

In specific embodiments, the topical composition includes a bioadhesive polymer system in 2.75±1 wt. %.

In specific embodiments, the topical composition includes a bioadhesive polymer system in 2.75±0.5 wt. %.

In specific embodiments, the topical composition includes a bioadhesive polymer system in 2.75±0.25 wt. %.

In specific embodiments, chitosan or hyaluronic acid is added at 0.2-0.5 wt. % for bioadhesion.

In specific embodiments, a bioadhesive hydrogel with >30% longer retention time than comparable non-bioadhesive formulations supports daily application.

### Personalization and Regimen Optimization Technologies

### Modular Multi-Chambered Dispensing and Dose Personalization

Specific embodiments of the present invention address variation in consumer needs, hair types, and clinical scenarios with custom mixing at point-of-use; minimizes excess costs and SKUs. Such embodiments permit selectable ratios and sequence of actives, penetration enhancers, and conditioning agents.

In specific embodiments, kits include modular dispensers with separate reservoirs for APIs and excipients.

In specific embodiments, users select ratios of finasteride/minoxidil/dutasteride before mixing and application.

In specific embodiments, a triple-cartridge bottle dispenses APIs at user-set 1:2:1 ratios and allows stepwise scalp application.

### Sensor-Guided or Feedback-Optimized Regimen Scheduling

Specific embodiments of the present invention improve treatment outcomes, compliance, and supports “smart” and digital health business models; provide regulatory advantages as a medical device-drug combination. Such embodiments integrate sensors or companion apps for monitoring dosage adherence, scalp humidity/pH, and uses machine learning for regimen adjustment.

In specific embodiments, digital sensors or mobile apps monitor scalp parameters and dynamically optimize the regimen.

In specific embodiments, hydration sensors trigger API release only above a moisture threshold.

In specific embodiments, a Bluetooth-enabled dispenser logs user application data and transmits to an app for trichogram-based regimen changes.

### Device-Assisted and Energy-Triggered Formulations

### Device-Assisted Delivery Methods (Microneedling, LLLT, Ultrasound)

Specific embodiments of the present invention deliver superior follicular absorption and clinical efficacy, and supports bundled device-formulation commercial strategies. Such embodiments provide for the use of companion devices—microneedle arrays, true-laser LLLT, ultrasound patches—creates synergistic effects and improves outcomes.

In specific embodiments, topical compositions are administered with device-assisted delivery.

In specific embodiments, microneedling rollers (192 needles, 0.3 mm) are included in the kit and used pre-application.

In specific embodiments, a patch containing dissolvable microneedle arrays preloaded with minoxidil is applied weekly for overnight release.

### Energy-Activated or Triggered API Release

Specific embodiments of the present invention allow for “on-demand” dosing to synchronize with consumer or clinical schedules; offers claimable activation mechanism for exclusivity. With such embodiments, photo- or heat-activated carrier systems selectively release APIs at specific wavelengths or temperatures.

In specific embodiments, active release is triggered by external energy (light/electrical).

In specific embodiments, APIs encapsulated in polymers release when exposed to 650-700 nm red light.

In specific embodiments, a self-heating pack integrated into the dispenser warms the formulation to 40° C., activating phase-change release of follicular-targeted APIs.

### Regenerative, Microbiome, and Anti-Inflammatory Formulations

### Exosome or Stem Cell Conditioned Media Additives

Specific embodiments of the present invention address regenerative medicine and growth factor markets. Such embodiments accelerate follicle recovery and new hair formation using bioactive vesicles or conditioned media.

In specific embodiments, exosomes or conditioned media are blended into the formulation.

In specific embodiments, 1-5% human adipose-derived exosome suspensions are mixed with minoxidil and penetration enhancers.

In specific embodiments, exosome-loaded matrices demonstrated to increase anagen phase follicles by >50% in 12-week studies.

### Probiotic or Prebiotic Scalp Additives

Specific embodiments of the present invention address trending holistic and microbiome health markets; support claimable microbiota modulation benefits. With such embodiments, prebiotic oligosaccharides or probiotic lysates reduce inflammation, support scalp health, and potentially synergize with active APIs.

In specific embodiments, probiotic or prebiotic additives are incorporated into the topical regimen.

In specific embodiments, Lactobacillus ferment lysate at 2% is added to the inventive composition described herein.

In specific embodiments, prebiotic blends are supplied in freeze-dried capsules reconstituted with API solution immediately before application.

### Dual-Action Antibacterial/Antifungal Systems

Specific embodiments of the present invention address persistent dandruff, seborrheic dermatitis, and regulatory requirement for antimicrobial activity in certain geographies. Such embodiments include zinc pyrithione, ketoconazole, and PHMB for antimicrobial control, scalp comfort, and enhanced follicular outcomes.

In specific embodiments, topical composition includes an antimicrobial or antifungal agent.

In specific embodiments, ketoconazole is present at 1-2% with minoxidil and penetration enhancer.

In specific embodiments, betaine-based antibacterial actives (0.5%) are combined with a slow-release gel containing minoxidil at 5%.

### Packaging, Stability, and User Experience Enhancements

### UV-Protective, Calibrated Dropper Packaging

Specific embodiments of the present invention reduce API photodegradation, increases shelf life, and supports regulatory claims for potency and stability. In such embodiments, pharmaceutical-grade glass and closures with UV-blocking layers deliver >40% less active degradation and >95% dosing accuracy.

In specific embodiments, kits comprise glass bottles, UV-protective closures, and calibrated droppers.

In specific embodiments, amber Type III glass bottles are paired with UV-blocking cap and 1.0 mL±0.05 mL dropper.

In specific embodiments, closure design includes a calibrated, UV-coated dropper reducing API loss by >40% over 6 months storage.

### Aroma-Functional Compliance Boosters

Specific embodiments of the present invention increase consumer satisfaction, adherence, and repeat purchase via evidence-based sensory additives. Such embodiments incorporate specific olfactory receptor agonists (menthol, SANDALORE™) with proven follicular activation and patient “liking.”

In specific embodiments, composition includes bioactive aroma compounds for compliance boosting.

In specific embodiments, SANDALORE™ (0.2%) and menthol (0.3%) are blended for dual function (mood/efficacy).

In specific embodiments, herbal aroma blend selected via consumer panel to maximize repeated use and regimen compliance.

### Waterless Concentrate and Freeze-Dried Single-Use Formats

Specific embodiments of the present invention enable portable, long-term stable, low-preservative products for direct-to-consumer, specialty retail, and global e-commerce with minimal shipping costs. In such embodiments, waterless and freeze-dried formats allow reconstitution at home, with ≥98% retained potency after 24 months.

In specific embodiments, composition is supplied as waterless concentrate or freeze-dried powder.

In specific embodiments, single-use capsules containing freeze-dried minoxidil and penetration enhancer are reconstituted before application.

In specific embodiments, travel-sized anhydrous gel (minoxidil 8%) is diluted with proprietary solution for on-the-go use.

### Immediate Cosmetic Coverage Additives (Hair Fibers, Color-Match)

Specific embodiments of the present invention provide instant visual improvement alongside therapeutic benefit; bridges medical and cosmetic “dual-action” categories. In such embodiments, color-matched keratin fibers, electrostatically charged powders, and volumizing sprays included as part of regimen kits.

In specific embodiments, kit includes color-matched hair fibers or volumizing powder with API compositions.

In specific embodiments, electrostatic keratin fibers included in kit adhere immediately to the scalp post-API application.

In specific embodiments, 10 shades of fiber powder are packaged for matching regrowth therapy with natural hair color.

### Stepwise Follicle Priming Compositions

Specific embodiments of the present invention reference to “priming” step in protocol supports outcome improvements, clinical claims, and structured regimen products. In such embodiments, follicle priming agents (caffeine, low-dose retinoic acid, acids) are applied before main API dose to improve uptake.

In specific embodiments, stepwise protocol applies follicle-priming agent before main API composition.

In specific embodiments, regimen includes topical caffeine 2% 10 minutes before API application.

In specific embodiments, dual-vial kit delivers glycolic acid pre-treatment followed by minoxidil/finasteride gel.

### Scalp Health, Barrier Restoration, and Responsive Carriers

### Scalp Barrier Restoration Agents

Specific embodiments of the present invention satisfy regulatory and consumer demand for scalp tolerance, reduced irritancy, prolonged use, and repeat purchasing. In such embodiments, ceramides, cholesterol, and panthenol as recovery/add-on agents minimize stratum corneum disruption and accelerate healing.

In specific embodiments, scalp barrier restoration agents are included in topical regimen.

In specific embodiments, lotion with ceramide NP 0.1% is applied 60 minutes post-API application.

In specific embodiments, panthenol-containing lotion is released from matrix embedded in the main API gel after 1 hour delay.

### Smart-Responsive Polymer Carrier Systems

Specific embodiments of the present invention enable dynamic “on demand” or responsive dosing for highest efficacy and safety; supports device-claimable products. In such embodiments, humidity- or sebum-sensitive hydrogels modulate API release as scalp conditions warrant, minimizing unnecessary exposure.

In specific embodiments, carrier polymers release API in response to humidity or scalp sweat.

In specific embodiments, hydrogel matrices expand to release API when scalp moisture rises above 50%, as measured by onboard sensor.

In specific embodiments, temperature-responsive polymer in finasteride gel pulses API dosing only when scalp temperature exceeds 32° C.

### Regenerative Scaffold Technologies

### Regenerated Bioactive Scaffold for Follicle Support

Specific embodiments of the present invention allow for patentable differentiation, advanced clinical claims, and modular “regrowth support” technologies for next-gen products. In such embodiments, biodegradable peptide or ECM-mimicking scaffolds form films or matrices supporting follicle structure and slow API release.

In specific embodiments, API is delivered in a scaffold forming film/matrix after application.

In specific embodiments, the peptide-based scaffold persists 24-72 hours and degrades in synchrony with follicle cycles.

In specific embodiments, cross-linked ECM-mimetic film is formed following minoxidil application, spatially localizing APIs to miniaturized follicles for ≥48 hours.

### Genetic Screening and Precision Medicine Integration

### Companion Genetic Testing and Phenotype-Based Protocols

Specific embodiments of the present invention support precision medicine claims, enables individualized insurance reimbursement, and meets emerging consumer expectations. In such embodiments, kits are paired with buccal swab DNA tests for AR/WNT10A genotyping, with app-guided recommendations.

In specific embodiments, kit includes genetic screening means for regimen optimization.

In specific embodiments, app-linked buccal swab allows AR or WNT10A genotype-based dose personalization.

In specific embodiments, app recommends specific minoxidil/dutasteride/penetration enhancer ratios upon receiving user genetic report.

### Advanced Bioadhesive Matrix System

Specific embodiments of the present invention provide for a bioadhesive topical delivery that significantly increases scalp retention, improving API efficacy, lowering dosing frequency, and enhancing consumer experience—addressing market needs for user-friendly, low-wastage solutions and differentiable product formats (e.g., gels, patches). Such embodiments include polymers (e.g., chitosan, hyaluronic acid) providing prolonged scalp contact and controlled release, outperforming standard solutions that run off or evaporate quickly.

In specific embodiments, the topical composition includes a bioadhesive matrix to increase scalp residence time of actives.

In specific embodiments, a hydrogel comprising 0.2-0.5% chitosan or hyaluronic acid is used, which retains ≥30% longer than non-bioadhesive formulations.

In specific embodiments, the active ingredient (e.g., minoxidil (8% w/w) and finasteride (0.3% w/w)) are embedded in a chitosan-based gel that adheres for at least 60 minutes, enabling once-daily dosing.

### Multi-Chambered, Modular, or Sequential Dispensing Packaging

Specific embodiments of the present invention provide for user-customizable dosing, combination therapies, and targeted delivery for varying patterns of hair loss—an industry trend toward personalized regimens and SKU minimization. Such embodiments prevent or mitigate ingredient incompatibility before application; enables sequential administration (e.g., priming+main API) for optimal absorption.

In specific embodiments, the kit includes multi-chambered or modular dispensing systems for separation and customizable mixing at point of use.

In specific embodiments, a dual-cartridge bottle dispenses minoxidil and finasteride separately for mixing prior to application.

In specific embodiments, the kit features a triple-chambered dispenser that allows user-selectable dosing ratios of finasteride, minoxidil, and dutasteride, with instructions for specific hair loss types.

### Integration of Scalp Diagnostic Feedback Devices

Specific embodiments of the present invention provide for smart regimens driven by real-time user data (moisture, pH, inflammation) that align with precision medicine trends and digital health, increasing user engagement, compliance, and clinical performance. Such embodiments allow for regimen optimization based on actual scalp condition, reducing side effects and maximizing efficacy compared to standard fixed regimens.

In specific embodiments, the kit is provided with a scalp diagnostic feedback device to guide regimen scheduling or ingredient selection.

In specific embodiments, a wearable sensor continuously tracks scalp pH and hydration, with companion app integration recommending dosing and formulation adjustments.

In specific embodiments, the dispenser includes a colorimetric strip that changes upon detection of scalp inflammation, prompting adjunct anti-inflammatory treatment with the main regimen.

### Device-Assisted Delivery (Microneedling, LLLLT, Ultrasound)

Specific embodiments of the present invention provide for adjunct devices included in kit (microneedling rollers, true-laser LLLT, ultrasound patches) that generate superior market differentiation and synergize with topical products for improved outcomes—an established competitive strategy among advanced hair regrowth therapies. Such embodiments produce significantly higher follicular absorption of actives with reduced dosing, supporting bundled device-formulation commercial strategies.

In specific embodiments, the regimen integrates device-assisted delivery to enhance topical API penetration.

In specific embodiments, the kit includes a 0.3 mm microneedling roller for use before or after API application, as part of the treatment protocol.

In specific embodiments, a dissolvable microneedle patch loaded with minoxidil and laurocapram is applied weekly for overnight follicular release, improving regrowth compared to solution alone.

### Exosome or Stem Cell Conditioned Media Addition

Specific embodiments of the present invention add a regenerative medicine angle (stem cell, exosome therapies), aligning with “next-generation” aesthetics and clinical trends for follicle recovery and new hair formation, appealing to premium market segments. Such embodiments support follicle regeneration; provide synergistic effect with existing APIs, addressing a broader range of hair loss etiologies beyond pure DHT blockade.

In specific embodiments, the composition is blended with stem cell-conditioned media or exosomes to enhance follicle regeneration.

In specific embodiments, human adipose-derived exosomes (1-5% w/w) are included in the formulation alongside minoxidil and a penetration enhancer.

In specific embodiments, exosomes (0.5% w/w) are integrated into a minoxidil-chitosan gel, demonstrated to increase anagen phase follicles as confirmed by trichoscopy within 12 weeks.

### Heat-Activated or Thermoresponsive Formulations

Specific embodiments of the present invention provide for products that activate on skin contact add premium features and can command higher pricing; “on-demand” API release matches clinical needs and active lifestyle consumer preferences. In such embodiments, phase-change gels or mild exothermic systems improve local delivery timing, increase follicular penetration, and avoid surface wastage.

In specific embodiments, the topical composition undergoes a phase transition or releases API upon exposure to elevated temperature.

In specific embodiments, a phase-change hydrogel containing minoxidil transitions from liquid to semi-solid above 32° C., increasing scalp retention by >25%.

In specific embodiments, a self-heating pack warms the formulation to 40° C. upon mixing, activating phase-change release of active ingredient (e.g., minoxidil and/or finasteride) for enhanced follicular absorption within 30 minutes of application.

### Controlled-Release (Microencapsulated or Nanoparticle) APIs

Specific embodiments of the present invention provide for sustained-release and microencapsulation technologies that support less frequent dosing, increased convenience, and competitive claims for efficacy and safety; they are increasingly requested in cosmetics and pharmaceuticals. In such embodiments, microencapsulated APIs improve stability, slow-release kinetics, and target follicular uptake over bulk APIs.

In specific embodiments, the active ingredient is supplied in microencapsulated or nanoparticle form for sustained or triggered release upon topical administration.

In specific embodiments, minoxidil and finasteride are microencapsulated in PLGA microspheres (10-100 μm) for slow release over 4-24 hours post-application.

In specific embodiments, solid lipid nanoparticles (100 nm diameter) loaded with minoxidil are used to increase follicular deposition by ≥50% (measured by Franz cell assay) and maintain chemical stability for ≥12 months.

### Inclusion of Antimicrobial/Antifungal Agents for Scalp Microbiome Health

Specific embodiments of the present invention provide for hair loss that frequently coexists with dandruff, seborrheic dermatitis, or fungal infections; combining regrowth and scalp health claims increases market appeal and broadens customer base. In such embodiments, agents like zinc pyrithione and ketoconazole control scalp biofilm and inflammation, supporting both therapeutic and preventive use.

In specific embodiments, the composition includes antimicrobial or antifungal ingredients to support scalp health and further enhance API efficacy.

In specific embodiments, the topical composition comprises minoxidil (5%), ketoconazole (1%), and zinc pyrithione (2%), ensuring both antifungal and hair regrowth effects.

In specific embodiments, a two-step regimen applies ketoconazole solution daily for one week, followed by twice-daily minoxidil/saw palmetto solution for regrowth and lasting microbiome control.

### Aroma-Functional Compliance Boosters

Specific embodiments of the present invention provide for evidence-based sensory additives such as SANDALORE™ and menthol that improve patient adherence, repeat purchase rates, and conversion for premium product lines, matching consumer demand for pleasurable self-care experiences. In such embodiments, these compounds activate olfactory receptors associated with follicle stimulation and enhanced mood, offering a dual benefit of efficacy and acceptability.

In specific embodiments, the topical formulation includes bioactive aroma compounds intended to enhance compliance and mood as well as efficacy.

In specific embodiments, SANDALORE™ (0.1-1%) and menthol (0.1-0.5%) are blended into the formulation, as selected via consumer panel for improved repeat usage.

In specific embodiments, a rosemary oil-caffeine—SANDALORE™-menthol solution is dispensed in amber glass bottles with a calibrated dropper, marketed as both regrowth and sensory compliance booster therapy.

### Personalized Regimen Scheduling Via Companion App or Machine Learning

Specific embodiments of the present invention provide for digital health tools (apps, Bluetooth devices, cloud-based regimen tracking) are used to increase product value, support subscription models, and/or reduce dropout rates by tailoring dosing and/or reminders. In such embodiments, dynamic, app-guided or device-monitored regimens optimize individual response vs. one-size-fits-all dosing; companion diagnostics and scheduled recommendations match cutting-edge pharma trends.

In specific embodiments, the kit includes a digital device, app, or wearable enabling personalized regimen scheduling and monitoring.

In specific embodiments, a Bluetooth-enabled dispenser connects to a companion app to track administration and recommend adjustments based on digital scalp analysis.

In specific embodiments, machine learning algorithms using real-time trichoscopy data adjust ingredient ratios and prompt the user, increasing scalp density improvement to at least 25% at 24 weeks as measured by in-app reporting.

### Multi-Chambered, Modular, or Sequential Dispensing Packaging

Specific embodiments of the present invention provide for multi-chambered and modular dispensing systems that enable personalized, flexible product offerings, minimize SKU proliferation, and allow consumer choice or regimen tailoring at point of use. These systems meet strong demand for personalized topical therapeutics, improve dosing accuracy, and support advanced treatment protocols (e.g., stepwise administration) that competitive products do not offer at scale. In such embodiments, these packaging solutions prevent ingredient incompatibility before use, reduce degradation risks, and support tailored therapy protocols (e.g., mixing APIs or penetration enhancers immediately before application). They also enable “on-demand” regimen adjustment and facilitate companion device integration (app, diagnostic, etc.) for smart health applications.

In specific embodiments, the invention comprises a topical hair regrowth kit equipped with multi-chambered or modular dispensing packaging that allows separation and customizable mixing of actives, penetration enhancers, and conditioning agents at the point of use.

In specific embodiments, the kit features a dual-cartridge bottle that dispenses minoxidil and finasteride from separate reservoirs, with user-selectable mixing and application prior to scalp administration.

In specific embodiments, a triple-chambered dispenser allows precise dosing ratios of finasteride (0.3%), minoxidil (8%), and dutasteride (0.3%), with instructions for mixing and sequential scalp application tailored per user or physician guidance.

### Device-Assisted Delivery and Energy-Activated Release Systems

Specific embodiments of the present invention provide for integration of microneedling, phototherapy (LLLT), ultrasound patches, and/or energy-activated release enables synergy between topical products and physical devices, providing substantial differentiation in efficacy and supporting premium, bundled product lines. These modalities match emerging clinical and consumer trends around combined therapy for superior follicular delivery. In such embodiments, synergistic application of device-assisted techniques (microneedling, LLLT, ultrasound, or microcurrent) amplifies follicular API uptake, enables lower dosing, and reduces systemic exposure. Energy-activated release systems offer controlled, “on-demand” dosing synchronized with consumer schedules or clinical protocols.

In specific embodiments, the invention comprises a topical regimen for hair regrowth utilizing device-assisted delivery—such as microneedling, phototherapy, ultrasound, or microcurrent devices—to enhance penetration and clinical outcomes.

In specific embodiments, a kit includes a 0.3 mm microneedling roller for pre-treatment, followed by topical application of a gel with APIs and penetration enhancer, repeated daily.

In specific embodiments, topical minoxidil and finasteride are embedded in a phase-change hydrogel that transitions from liquid to semi-solid above 32° C. and is activated by a self-heating pack during application, with enhanced follicular uptake measured within 30 minutes post-treatment.

### Bioadhesive Matrix Carrier Systems for Prolonged Follicular Delivery

Specific embodiments of the present invention provide for bioadhesive gels and matrices that prolong residence time, minimize runoff, and support sustained API release, enabling once-daily dosing and improved consumer experience. These carriers also differentiate products versus conventional solutions, responding to needs for effective follicular delivery, reduced waste, and higher efficacy. In such embodiments, embedding APIs in bioadhesive (e.g., chitosan, hyaluronic acid) polymers localize actives to scalp/follicles, increases follicular API uptake, and allow for reduced frequency and dosing. Bioadhesion confers “stick-and-stay” functionality, giving products superior retention compared to liquids or foams.

In specific embodiments, the invention comprises a bioadhesive gel or hydrogel carrier system for topical hair regrowth APIs, formulated to adhere to the scalp and release actives in a controlled manner.

In specific embodiments, a chitosan-based gel matrix containing minoxidil (8%) and finasteride (0.3%) provides ≥30% longer retention and delivers ≥60% of API to follicles within 20 minutes post-application.

In specific embodiments, APIs are embedded in a hyaluronic acid-chitosan hydrogel, with scalp adherence for at least 60 minutes, enabling once-daily dosing and enhanced trichoscopy-measured hair density.

### Integration of Regenerative Ingredients: Exosomes or Stem Cell Conditioned Media

Specific embodiments of the present invention provide for incorporation of exosome or stem cell-conditioned media components brings advanced regenerative capabilities and strong market appeal, addressing the premium hair restoration segment with claims on follicle regeneration, stem cell activation, and novel mechanisms of action that are not available in legacy products. In such embodiments, exosome- or stem cell-conditioned media ingredients can induce follicular regeneration, prolong anagen phase, and increase terminal hair density through biological signaling. Their combination with conventional APIs and penetration enhancers enables synergistic, multi-mechanism regrowth.

In specific embodiments, the invention comprises a topical hair regrowth formulation supplemented with exosomes or stem cell-conditioned media at 1-5% w/w to support follicle regeneration when administered to the scalp.

In specific embodiments, minoxidil (8%) and penetration enhancer (e.g., laurocapram) are blended with 2% adipose-derived exosome suspension and applied as a gel, resulting in increased anagen phase follicle counts in 12-week trichoscopy studies.

In specific embodiments, a bioadhesive hydrogel containing 0.5% exosomes and minoxidil (8%) is designed for daily application, confirmed to elevate anagen-to-telogen ratio by >50% compared to controls.

### Sensor-Guided or App-Optimized Personalized Regimen Scheduling

Specific embodiments of the present invention provide for integration of digital sensors, companion apps, and machine learning algorithms for regimen tracking, optimization, and compliance support is highly commercially viable. These features meet demand for smart therapeutics, drive user engagement, and support personalized dosing and formulation that responds to individual scalp conditions or genetic data, giving products a major market edge.

In such embodiments, sensor-guided and app-driven regimens allow for dynamic dose adjustment, adherence tracking, real-time scalp parameter monitoring, and evidence-based recommendations for ingredient ratios, application timing, or treatment modulation based on user biology and feedback.

In specific embodiments, the invention comprises a topical hair regrowth kit integrated with scalp diagnostic sensors and a companion app, enabling regimen scheduling and ingredient selection tailored to individual needs and feedback.

In specific embodiments, a Bluetooth-enabled dispenser collects dosing data and transmits scalp hydration and pH readings to a mobile app, prompting real-time adjustments in ingredient ratios and scheduling based on algorithmic analysis.

In specific embodiments, machine learning algorithms utilizing trichoscopy data and user input recommend daily minoxidil/finasteride/dutasteride ratios, dynamically adjusting treatment to maximize hair density improvement and minimize irritation, tracked through the companion app interface.

### Enumerated Embodiments

Specific enumerated embodiments <1> to <88> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Topical Composition

<Embodiment 1> A topical composition for promoting hair regrowth comprising an active pharmaceutical ingredient comprising at least one of finasteride, minoxidil, dutasteride, and latanoprost, a penetration enhancer, and a solvent system suitable for scalp application, wherein the penetration enhancer is present in an amount effective to increase follicular deposition of the active pharmaceutical ingredient compared to an identical composition lacking the penetration enhancer.

<Embodiment 2> A topical formulation comprising at least one hair regrowth agent and at least one penetration enhancer, wherein the formulation is processed to achieve a mass-median particle diameter (D90) of 8.5 μm or less for the hair regrowth agent, and a Span value of less than 1.7.

<Embodiment 3> A topical composition comprising (a) at least one of finasteride, minoxidil, dutasteride, or latanoprost; (b) retinoic acid; and (c) hydrocortisone, in a physiologically acceptable vehicle, for administration in combination with device-assisted delivery.

<Embodiment 4> A topical composition for hair regrowth, wherein the active pharmaceutical ingredient and penetration enhancer are embedded within a bioadhesive polymer matrix to increase residence time on the scalp compared to a non-bioadhesive system.

<Embodiment 5> A topical composition for treating hair loss, wherein the composition comprises a pharmaceutical agent comprising at least one of finasteride, minoxidil, dutasteride, and latanoprost, and further comprises exosomes or stem cell-conditioned media for follicle regeneration.

<Embodiment 6> A topical composition for promoting hair regrowth comprising an active pharmaceutical ingredient comprising at least one of finasteride, minoxidil, dutasteride, and latanoprost, a penetration enhancer, and a solvent system suitable for scalp application, wherein the penetration enhancer is present in an amount effective to increase follicular deposition of the active pharmaceutical ingredient compared to an identical composition lacking the penetration enhancer; wherein active pharmaceutical ingredients are microencapsulated in a biodegradable polymer material, providing controlled release of the active pharmaceutical ingredient over at least 4 hours post-application.

<Embodiment 7> A topical composition comprising finasteride and minoxidil in combination, a penetration enhancer comprising at least one of Transcutol®, laurocapram, and dimethyl isosorbide, and formulated so the composition achieves at least a 40% increase in follicular API deposition compared to an identical composition lacking the penetration enhancer.

<Embodiment 8> A topical hair regrowth composition, wherein the active ingredient particles have D90 ≤8.5 μm and are delivered in a solvent system further comprising a polymer comprising at least one of chitosan and hyaluronic acid, as a bioadhesive agent.

<Embodiment 9> A topical composition for promoting hair growth, comprising at least one active pharmaceutical ingredient, at least one penetration enhancer, and a pH buffer system configured to adjust the final composition pH to between 5.7 and 6.4 upon administration.

<Embodiment 10> A topical composition, wherein the active pharmaceutical ingredient is loaded in a slow-release hydrogel with a residence time on the scalp of at least 60 minutes, the hydrogel comprising 0.2-0.5% chitosan or hyaluronic acid.

<Embodiment 11> A topical composition for hair regrowth comprising minoxidil, at least one penetration enhancer, and ketoconazole at 1-2 wt %, wherein the formulation reduces scalp irritation and fungal growth.

<Embodiment 12> A topical composition for treating hair loss, comprising a hair regrowth agent, a penetration enhancer, and a prebiotic or probiotic additive comprising at least one of Lactobacillus ferment lysate and inulin, at concentrations of 0.5-5%.

<Embodiment 13> A topical composition comprising minoxidil encapsulated in a phase-change gel that transitions from liquid to semi-solid when exposed to temperatures above 32° C., increasing scalp retention by at least 25% compared to non-phase-change systems.

<Embodiment 14> A topical composition for hair regrowth supplied as a waterless concentrate or freeze-dried powder, reconstituted prior to use, with at least 95% retained active pharmaceutical ingredient potency after 24 months storage at 2-8° C.

<Embodiment 15> A topical composition for hair regrowth comprising minoxidil, a penetration enhancer comprising at least one of Transcutol®, laurocapram, and propylene glycol; wherein the penetration enhancer increases follicular minoxidil deposition by at least 40% as measured by a Franz cell diffusion assay.

<Embodiment 16> A topical composition comprising finasteride, minoxidil, retinoic acid, hydrocortisone, aloe vera, ethyl alcohol, propylene glycol, and a penetration enhancer, for reducing both hair loss and scalp irritation when administered daily.

<Embodiment 17> A topical composition comprising minoxidil and an antioxidant comprising at least one of BHT and EDTA, present in an amount effective to prevent at least 98% degradation of minoxidil under accelerated stability testing conditions.

<Embodiment 18> A topical hair regrowth composition comprising minoxidil, menthol, and SANDALORE™, wherein the sensory modifiers are present in amounts sufficient to enhance user compliance and mood during treatment.

<Embodiment 19> A topical composition for promoting hair regrowth comprising an active pharmaceutical ingredient comprising at least one of finasteride, minoxidil, dutasteride, and latanoprost, a penetration enhancer, and a solvent system suitable for scalp application, wherein the penetration enhancer is present in an amount effective to increase follicular deposition of the active pharmaceutical ingredient compared to an identical composition lacking the penetration enhance; that is formulated without known allergens, parabens, or sulfates, and verified to have a dermatological irritation score ≤1.0/4 on clinical patch testing.

<Embodiment 20> A topical composition for hair regrowth comprising minoxidil, aloe vera, hydrocortisone, and bisabolol, wherein the combination of agents provides at least a 30% reduction in scalp erythema versus minoxidil alone.

<Embodiment 21> A topical composition for promoting hair regrowth comprising an active pharmaceutical ingredient comprising at least one of finasteride, minoxidil, dutasteride, and latanoprost, a penetration enhancer, and a solvent system suitable for scalp application, wherein the penetration enhancer is present in an amount effective to increase follicular deposition of the active pharmaceutical ingredient compared to an identical composition lacking the penetration enhancer; wherein viscosity is maintained at 2,500±250 cP as measured by Brookfield RV DV-III Ultra, spindle #7, 20 rpm, optimized for controlled scalp application and retention.

<Embodiment 22> A topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; and (f) a penetration enhancer comprising at least one of Transcutol® (diethylene glycol monoethyl ether), cetyl alcohol, oleic acid, eucalyptol, polyethylene glycol, octyl salicylate, SiloxysSystem™ gel, laurocapram, dimethyl isosorbide (DMI), urea (carbamide), d-limonene, menthol, α-bisabolol, and Azone® (laurocapram).

<Embodiment 23> A topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; (f) at least one penetration enhancer; and at least one of (1)-(17): (1) co-solvent, (2) additional API, (3) vitamin/nutraceutical natural product, (4) antioxidant/chelator, (5) pH adjuster/buffer, (6) film-former & polymer, (7) conditioning agent, (8) CMC (cell membrane complex) mimic, (9) anti-inflammatory agent, (10) antimicrobial/preservative, (11) humectant, (12) sensory modifier, (13) penetration modulator, (14) stabilizer, (15) color stabilizer, (16) chelating agent, and (17) emulsifier/surfactant.

<Embodiment 24> A topical composition that includes: (a) at least one of finasteride, minoxidil, dutasteride, and latanoprost; (b) retinoic acid; (c) hydrocortisone; (d) aloe vera; (e) at least one of propylene glycol, ethanol, and water; and (f) at least one penetration enhancer. The composition may further include an additional active comprising at least one bimatoprost (0.005-0.03 wt. %), clascoterone (0.5-5 wt. %), cyclosporine (0.05-0.1 wt. %), pyrilutamide (0.1-0.5 wt. %), and any pharmaceutically acceptable salt thereof, individually or in combination, in concentrations chosen for maximal efficacy and minimum irritation, based on published clinical data or new empirical determinations presented herein.

<Embodiment 25> A topical composition that includes: (a) at least one of finasteride, minoxidil, dutasteride, and latanoprost; (b) retinoic acid; (c) at least one of hydrocortisone and aloe vera; (d) at least one of propylene glycol, ethanol, and water; and (e) penetration enhancer comprising at least one of Transcutol® (diethylene glycol monoethyl ether), cetyl alcohol, oleic acid, eucalyptol, polyethylene glycol, octyl salicylate, SiloxysSystem™ gel, laurocapram, dimethyl isosorbide (DMI), urea (carbamide), d-limonene, menthol, α-bisabolol, and Azone® (laurocapram).

<Embodiment 26> A topical composition that includes: (a) at least one of finasteride, minoxidil, dutasteride, and latanoprost; (b) retinoic acid; (c) at least one of hydrocortisone and aloe vera; (d) at least one of propylene glycol, ethanol, and water; (e) at least one penetration enhancer; and at least one of (1)-(17): (1) co-solvent, (2) additional API, (3) vitamin/nutraceutical natural product, (4) antioxidant/chelator, (5) pH adjuster/buffer, (6) film-former & polymer, (7) conditioning agent, (8) CMC (cell membrane complex) mimic, (9) anti-inflammatory agent, (10) antimicrobial/preservative, (11) humectant, (12) sensory modifier, (13) penetration modulator, (14) stabilizer, (15) color stabilizer, (16) chelating agent, and (17) emulsifier/surfactant.

<Embodiment 27> A topical composition that includes: (a) at least one of finasteride, minoxidil, dutasteride, and latanoprost; (b) retinoic acid; (c) at least one of hydrocortisone and aloe vera; (d) at least one of propylene glycol, ethanol, and water; and (f) at least one penetration enhancer. The composition may further include an additional active comprising at least one of bimatoprost, clascoterone, cyclosporine, pyrilutamide, and any pharmaceutically acceptable salt thereof, as well as any combination of the above, in amounts sufficient to promote hair regrowth, reduce irritation, or facilitate follicular penetration, as described throughout this specification.

### Method of Use

<Embodiment 28> A method for promoting hair regrowth in a human afflicted with hair loss or at risk thereof, comprising topically administering the composition of any one of <Embodiment 1> to <Embodiment 27> to the scalp at least once daily for a period sufficient to achieve an increase in hair density.

<Embodiment 29> A method for treating alopecia or androgenetic alopecia in a subject in need thereof, comprising applying to the scalp the composition of any one of <Embodiment 1> to <Embodiment 27> at a therapeutically effective frequency.

<Embodiment 30> A method for increasing the anagen-to-telogen hair ratio in a human, comprising topically administering the composition of any one of <Embodiment 1> to <Embodiment 27> to the scalp over a course of treatment.

<Embodiment 31> A method for reducing hair shedding in a human subject, comprising administering the composition of any one of <Embodiment 1> to <Embodiment 27> to the scalp at least once daily.

<Embodiment 32> A method for preventing hair loss in a subject at risk thereof, comprising topical administration of the composition of any one of <Embodiment 1> to <Embodiment 27> to the scalp following a predetermined regimen.

<Embodiment 33> A method for increasing hair shaft diameter in a human, comprising topical application of the composition of any one of <Embodiment 1> to <Embodiment 27> to the affected scalp area, repeated over at least 12 weeks.

<Embodiment 34> A method for increasing total scalp hair count, comprising administering the composition of any one of <Embodiment 1> to <Embodiment 27> to the scalp daily for a period of effective treatment duration.

<Embodiment 35> A method for treating female pattern hair loss, comprising topically administering the composition of any one of <Embodiment 1> to <Embodiment 27> to the scalp of a female patient according to a scheduled protocol.

<Embodiment 36> A method for improving scalp coverage, comprising administering the composition of any one of <Embodiment 1> to <Embodiment 27> topically to the scalp of a human subject suffering from reduced hair density.

<Embodiment 37> A method for prolonging the anagen phase of hair follicles in a human, comprising topical administration of the composition of any one of <Embodiment 1> to <Embodiment 27> in an effective amount to the scalp.

<Embodiment 38> A method for promoting hair regrowth in a human subject wherein the composition of any one of <Embodiment 1> to <Embodiment 27> is administered topically to the scalp in combination with microneedling at a needle length of 0.3 mm, once per week for at least 12 consecutive weeks.

<Embodiment 39> A method for increasing follicular deposition of active pharmaceutical ingredients in a human, comprising topically administering the composition of any one of <Embodiment 1> to <Embodiment 27> to the scalp, wherein said composition has a D90 particle size of ≤8.5 μm and is applied following a stepwise follicle-priming protocol with topical caffeine.

<Embodiment 40> A method for promoting hair regrowth in a human subject using a regimen comprising daily application of the composition of any one of <Embodiment 1> to <Embodiment 27>, wherein said regimen further includes monitoring scalp pH using a sensor to adjust administration timing.

<Embodiment 41> A method for reducing scalp DHT levels by at least 60% in a subject with androgenetic alopecia, comprising daily topical application of the composition of any one of <Embodiment 1> to <Embodiment 27> and co-administration of oral finasteride.

<Embodiment 42> A method for increasing anagen phase follicle counts by at least 50% within 12 weeks in a human patient, comprising topical administration of the composition of any one of <Embodiment 1> to <Embodiment 27>, said composition containing 1-5% exosome suspension.

<Embodiment 43> A method for promoting hair regrowth in a subject, wherein the composition of any one of <Embodiment 1> to <Embodiment 27> is applied topically and a companion app is used to schedule and log each administration.

<Embodiment 44> A method for treating hair loss in a subject with WNT10A gene polymorphism, comprising daily administration of the composition of any one of <Embodiment 1> to <Embodiment 27> to the subject's scalp.

<Embodiment 45> A method for promoting hair regrowth in a human subject, wherein the composition of any one of <Embodiment 1> to <Embodiment 27> is administered as a phase-change gel, the gel transitioning from liquid to semi-solid above 32° C. on skin contact.

<Embodiment 46> A method for treating hair loss and simultaneously reducing scalp erythema by at least 30% in a human, comprising topical administration of the composition of any one of <Embodiment 1> to <Embodiment 27> vcontaining both aloe vera and bisabolol.

<Embodiment 47> A method for increasing scalp hair density by at least 25% in a human subject with female pattern hair loss, comprising daily topical administration of the composition of any one of <Embodiment 1> to <Embodiment 27> and tracking progress using trichoscopy.

<Embodiment 48> A method for enhancing follicular penetration and hair regrowth in a human subject, comprising daily application of the composition of any one of <Embodiment 1> to <Embodiment 27> to the scalp, wherein the composition contains at least one penetration enhancer effective to increase follicular deposition by at least 40% compared to a control lacking penetration enhancer.

<Embodiment 49> A method for treating androgenetic alopecia in a male subject, comprising administering the composition of any one of <Embodiment 1> to <Embodiment 27> to the subject's scalp daily for at least 24 weeks.

<Embodiment 50> A method for promoting hair regrowth and scalp health in a human subject, comprising topically administering the composition of any one of <Embodiment 1> to <Embodiment 27> to the scalp, wherein the composition includes at least one antimicrobial or anti-inflammatory ingredient.

<Embodiment 51> A method for maintaining chemical stability of active pharmaceutical ingredients during treatment of hair loss, comprising topically administering the composition of any one of <Embodiment 1> to <Embodiment 27> containing an antioxidant effective to reduce API degradation by at least 98% under accelerated stability conditions.

<Embodiment 52> A method for increasing hair shaft diameter and tensile strength in a human subject, comprising daily topical administration of the composition of any one of <Embodiment 1> to <Embodiment 27> to the affected scalp area.

<Embodiment 53> A method for promoting hair regrowth in a human subject, wherein the composition of any one of <Embodiment 1> to <Embodiment 27> is administered topically and is formulated as a bioadhesive hydrogel to increase residence time on the scalp.

<Embodiment 54> A method for reducing epidermal retention time of active pharmaceutical ingredients in a subject, comprising topical administration of the composition of any one of <Embodiment 1> to <Embodiment 27>, which includes at least one penetration enhancer effective to reduce epidermal retention time to <15 minutes.

<Embodiment 55> A method for promoting hair density in a human subject, comprising daily administration of the composition of any one of <Embodiment 1> to <Embodiment 27> for at least 6 months, wherein the composition increases hair count by at least 45 hairs/cm².

<Embodiment 56> A method for improving user compliance with a hair regrowth regimen, comprising administering the composition of any one of <Embodiment 1> to <Embodiment 27> topically to a human scalp, wherein said composition contains a sensory modifier comprising at least one of menthol and SANDALORE™

<Embodiment 57> A method for treating hair loss in a subject by administering the composition of any one of <Embodiment 1> to <Embodiment 27> topically to the scalp and measuring patient-reported outcome (PRO) improvements via validated dermatological quality-of-life questionnaires.

<Embodiment 58> A method for promoting terminal hair regeneration in a human subject, comprising topically administering the composition of any one of <Embodiment 1> to

<Embodiment 27> in a regimen synchronized with low-level light therapy (LLLT) of 650-830 nm wavelength, at least twice per week.

### Kit

<Embodiment 59> A kit comprising: (i) the topical composition of any one of <Embodiment 1> to <Embodiment 27>; and (ii) a pharmaceutical-grade container suitable for storage and dispensing of said composition.

<Embodiment 60> A kit comprising: (i) the topical composition of any one of <Embodiment 1> to <Embodiment 27>; and (ii) a calibrated applicator configured to deliver a reproducible aliquot of said composition to the scalp.

<Embodiment 61> A kit comprising: (i) the topical composition of any one of <Embodiment 1> to <Embodiment 27>; (ii) instructions for use describing topical administration of said composition for promoting hair regrowth.

<Embodiment 62> A kit comprising: (i) the topical composition of any one of <Embodiment 1> to <Embodiment 27>; and (ii) a closure comprising a UV-protective coating configured to reduce photodegradation of actives in said composition by at least 40% over six months.

<Embodiment 63> A kit comprising: (i) the topical composition of any one of <Embodiment 1> to <Embodiment 27>; and (ii) a multi-chambered dispenser configured to separately store two or more components prior to application.

<Embodiment 64> A kit comprising: (i) the topical composition of any one of <Embodiment 1> to <Embodiment 27>; and (ii) a dropper calibrated to deliver 1.0 mL+0.05 mL of said composition per actuation.

<Embodiment 65> A kit comprising: (i) the topical composition of any one of <Embodiment 1> to <Embodiment 27>; and (ii) supplementary instructions including scalp diagnostic guidance for optimizing dosage scheduling based on user feedback.

<Embodiment 66> A kit comprising: (i) the topical composition of any one of <Embodiment 1> to <Embodiment 27>; and (ii) an additional item comprising at least one of a microneedling device, a low-level light therapy apparatus, and a scalp sensor.

<Embodiment 67> A kit comprising: (i) the topical composition of any one of <Embodiment 1> to <Embodiment 27>; and (ii) electronic compliance monitoring means configured to record and report user dosing history during administration.

<Embodiment 68> A kit comprising: (i) the topical composition of any one of <Embodiment 1> to <Embodiment 27>; and (ii) external packaging comprising at least one of single-use ampoules, multi-dose vials, or freeze-dried capsules supplied for reconstitution prior to use.

<Embodiment 69> A kit comprising: (i) the topical composition of any one of <Embodiment 1> to <Embodiment 27>, wherein the composition is formulated as a bioadhesive hydrogel containing chitosan (0.2-0.5% w/w); and (ii) a microneedling roller device comprising 192 needles of 0.3 mm for pre-application follicle priming.

<Embodiment 70> A kit comprising: (i) the topical composition of any one of <Embodiment 1> to <Embodiment 27>; (ii) a dual-chamber dispenser configured to separate a follicle-priming agent and a regrowth phase composition, with instructions for sequential scalp administration.

<Embodiment 71> A kit comprising: (i) the topical composition of any one of <Embodiment 1> to <Embodiment 27> with a pH-modifying buffer system; and (ii) a colorimetric strip integrated with the dispenser, configured to indicate scalp pH and prompt formulation adjustments.

<Embodiment 72> A kit comprising: (i) the topical composition of any one of <Embodiment 1> to <Embodiment 27> comprising exosomes (1-5% w/w); and (ii) a companion app configured for regimen scheduling and progress tracking via trichoscopy image analysis.

<Embodiment 73> A kit comprising: (i) the topical composition of any one of <Embodiment 1> to <Embodiment 27>; (ii) a phase-change hydrogel dispenser configured to activate API release at scalp temperatures above 32° C.

<Embodiment 74> A kit comprising: (i) the topical composition of any one of <Embodiment 1> to <Embodiment 27> wherein actives are encapsulated in PLGA microspheres; (ii) a slow-release applicator configured to deliver sustained dosing over at least four hours.

<Embodiment 75> A kit comprising: (i) the topical composition of any one of <Embodiment 1> to <Embodiment 27>, wherein said composition includes an antimicrobial agent comprising at least one of zinc pyrithione and ketoconazole; (ii) a dispenser incorporating an anti-contamination barrier.

<Embodiment 76> A kit comprising: (i) the topical composition of any one of <Embodiment 1> to <Embodiment 27>; (ii) a multi-cartridge bottle configured to allow user customization of finasteride, minoxidil, and dutasteride concentrations immediately prior to application.

<Embodiment 77> A kit comprising: (i) the topical composition of any one of <Embodiment 1> to <Embodiment 27>; (ii) a wearable scalp hydration sensor, and (iii) instructions for use detailing dosing adjustments based on real-time sensor readings.

<Embodiment 78> A kit comprising: (i) the topical composition of any one of <Embodiment 1> to <Embodiment 27> formulated as a freeze-dried powder in single-use capsules; (ii) a reconstitution vial containing USP purified water; and (iii) a calibrated mixing tool.

<Embodiment 79> A kit comprising: (i) the topical composition of any one of <Embodiment 1> to <Embodiment 27>; (ii) an amber glass bottle formatted for 40-75 mL capacity; and (iii) a dropper device with UV-blocking cap and dosage marks.

<Embodiment 80> A kit comprising: (i) the topical composition of any one of <Embodiment 1> to <Embodiment 27>; (ii) a patch delivery system incorporating bioadhesive hydrogel and integrated microarray needles preloaded with active agent.

<Embodiment 81> A kit comprising: (i) the topical composition of any one of <Embodiment 1> to <Embodiment 27>; (ii) a modular dispenser enabling point-of-use selection and mixing of at least one penetration enhancer strength.

<Embodiment 82> A kit comprising: (i) the topical composition of any one of <Embodiment 1> to <Embodiment 27>; (ii) a companion genetic screening tool comprising buccal swab and instructions for in-home testing; and (iii) app-based recommendations for regimen personalization.

<Embodiment 83> A kit comprising: (i) the topical composition of any one of <Embodiment 1> to <Embodiment 27> including menthol and SANDALORE™; and (ii) a color-matched keratin fiber powder for immediate cosmetic coverage, supplied in at least five human hair shades.

<Embodiment 84> A kit comprising: (i) the topical composition of any one of <Embodiment 1> to <Embodiment 27>; (ii) stepwise regimen instructions detailing the use of follicle-priming composition followed by main API application; and (iii) a time-release scalp barrier restoration lotion supplied for post-treatment.

<Embodiment 85> A kit comprising: (i) the topical composition of any one of <Embodiment 1> to <Embodiment 27>; (ii) a Bluetooth-enabled dispenser transmitting dosing data and scalp metrics to a mobile app for treatment optimization.

<Embodiment 86> A kit comprising: (i) the topical composition of any one of <Embodiment 1> to <Embodiment 27>; (ii) an applicator comprising both dropper and spray functions; and (iii) packaging including multi-lingual instructions for different global markets.

<Embodiment 87> A kit comprising: (i) the topical composition of any one of <Embodiment 1> to <Embodiment 27>; (ii) an electronic guidance system configured to prompt regimen scheduling, dose logging, and adverse reaction alerts during topical administration.

<Embodiment 88> A kit comprising: (i) the topical composition of any one of <Embodiment 1> to <Embodiment 27>; (ii) a multi-dose applicator configured to separately dispense said composition and supplementary scalp conditioning agent, with user instructions for cyclical administration based on scalp health status.

### EXAMPLES

All ingredients are combined under inert (N₂) atmosphere at <25° C., using a high-shear mixer (2000 rpm), added in order of solubility (e.g., minoxidil/finasteride/retinoic acid in alcohol first; then hydrocortisone; all excipients added at <30° C.), to optimize dissolution and minimize degradation.

### Production Example 1: Topical Finasteride & Minoxidil

| Ingredient | Function |
|---|---|
| Finasteride (0.3 wt. %) | Active ingredient. Stops hair loss and promotes growth by |
| | blocking Dihydrotestosterone (DHT) |
| Minoxidil (8.0 wt. %) | Active ingredient. Regrows hair and prolongs the growth |
| | (anagen) phase of hair follicles |
| Retinoic acid (0.001 wt. %) | Active ingredient. Promotes denser hair regrowth |
| Hydrocortisone (1.0 wt. %) | Active ingredient. Prevents irritation and redness |
| Aloe Vera | Inactive ingredient. Helps calm irritation that may be caused |
| | by active ingredients. Also helps to reduce inflammation. |
| Propylene Glycol | Inactive ingredient. Alcohol base that retains moisture and acts |
| | as a carrier (solvent) for the active ingredients above. |
| Ethyl Alcohol | Inactive ingredient. Naturally-derived plant alcohol that acts as |
| | a carrier (solvent) for the active ingredients above. |

### Production Example 2: Topical Dutasteride & Minoxidil

| Ingredient | Function |
|---|---|
| Dutasteride (0.3 wt. %) | Active ingredient. Stops hair loss and regrows hair by blocking |
| | Dihydrotestosterone (DHT), a common factor in hair loss. |
| | Dutasteride works by inhibiting both type 1 and type 2 5-alpha |
| | reductase, which converts Testosterone to DHT. |
| Minoxidil (8.0 wt. %) | Active ingredient. Regrows hair and prolongs the growth |
| | (anagen) phase of hair follicles |
| Retinoic acid (0.001 wt. %) | Active ingredient. Promotes denser hair regrowth |
| Hydrocortisone (1.0 wt. %) | Active ingredient. Prevents irritation and redness |
| Aloe Vera | Inactive ingredient. Helps calm irritation that may be caused |
| | by active ingredients. Also helps to reduce inflammation. |
| Propylene Glycol | Inactive ingredient. Alcohol base that retains moisture and acts |
| | as a carrier (solvent) for the active ingredients above. |
| Ethyl Alcohol | Inactive ingredient. Naturally-derived plant alcohol that acts as |
| | a carrier (solvent) for the active ingredients above. |

### Production Example 3: Topical Finasteride, Dutasternde & Minoxidil

| Ingredient | Function |
|---|---|
| Finasteride (0.1 wt. %) | Active ingredient. Stops hair loss and promotes growth by |
| | blocking Dihydrotestosterone (DHT) |
| Dutasteride (0.1 wt. %) | Active ingredient. Stops hair loss and regrows hair by blocking |
| | Dihydrotestosterone (DHT), a common factor in hair loss. |
| | Dutasteride works by inhibiting both type 1 and type 2 5-alpha |
| | reductase, which converts Testosterone to DHT. |
| Minoxidil (8.0 wt. %) | Active ingredient. Regrows hair and prolongs the growth |
| | (anagen) phase of hair follicles |
| Retinoic acid (0.001 wt. %) | Active ingredient. Promotes denser hair regrowth |
| Hydrocortisone (1.0 wt. %) | Active ingredient. Prevents irritation and redness |
| Aloe Vera | Inactive ingredient. Helps calm irritation that may be caused |
| | by active ingredients. Also helps to reduce inflammation. |
| Propylene Glycol | Inactive ingredient. Alcohol base that retains moisture and acts |
| | as a carrier (solvent) for the active ingredients above. |
| Ethyl Alcohol | Inactive ingredient. Naturally-derived plant alcohol that acts as |
| | a carrier (solvent) for the active ingredients above. |

### Production Example 4: Topical Minoxidil

| Ingredient | Function |
|---|---|
| Minoxidil (8.0 wt. %) | Active ingredient. Regrows hair and prolongs the growth |
| | (anagen) phase of hair follicles |
| Retinoic acid (0.001 wt. %) | Active ingredient. Promotes denser hair regrowth |
| Hydrocortisone (1.0 wt. %) | Active ingredient. Prevents irritation and redness |
| Aloe Vera | Inactive ingredient. Helps calm irritation that may be caused |
| | by active ingredients. Also helps to reduce inflammation. |
| Propylene Glycol | Inactive ingredient. Alcohol base that retains moisture and acts |
| | as a carrier (solvent) for the active ingredients above. |
| Ethyl Alcohol | Inactive ingredient. Naturally-derived plant alcohol that acts as |
| | a carrier (solvent) for the active ingredients above. |

### Production Example 5: Topical Finasteride & Minoxidil (Aqueous)

| Ingredient | Function |
|---|---|
| Finasteride (0.25 wt. %) | Active ingredient. Stops hair loss and promotes growth by |
| | blocking Dihydrotestosterone (DHT) |
| Minoxidil (8.0 wt. %) | Active ingredient. Regrows hair and prolongs the growth |
| | (anagen) phase of hair follicles |
| Retinoic acid (0.001 wt. %) | Active ingredient. Promotes denser hair regrowth |
| Hydrocortisone (1.0 wt. %) | Active ingredient. Prevents irritation and redness |
| Aloe Vera | Inactive ingredient. Helps calm irritation that may be caused |
| | by active ingredients. Also helps to reduce inflammation. |
| Propylene Glycol | Inactive ingredient. Alcohol base that retains moisture and acts |
| | as a carrier (solvent) for the active ingredients above. |
| Ethyl Alcohol | Inactive ingredient. Naturally-derived plant alcohol that acts as |
| | a carrier (solvent) for the active ingredients above. |

### Production Example 6: Topical Latanoprost, Finasteride & Minoxidil

| Ingredient | Function |
|---|---|
| Latanoprost (0.005 wt. %) | Active ingredient. Increases hair density and stimulates healthy |
| | hair follicle activity. |
| Finasteride (0.3 wt. %) | Active ingredient. Stops hair loss and promotes growth by |
| | blocking Dihydrotestosterone (DHT) |
| Minoxidil (8.0 wt. %) | Active ingredient. Regrows hair and prolongs the growth |
| | (anagen) phase of hair follicles |
| Retinoic acid (0.001 wt. %) | Active ingredient. Promotes denser hair regrowth |
| Hydrocortisone (1.0 wt. %) | Active ingredient. Prevents irritation and redness |
| Aloe Vera | Inactive ingredient. Helps calm irritation that may be caused |
| | by active ingredients. Also helps to reduce inflammation. |
| Propylene Glycol | Inactive ingredient. Alcohol base that retains moisture and acts |
| | as a carrier for the active ingredients above. |
| Ethyl Alcohol | Inactive ingredient. Naturally-derived plant alcohol that acts as |
| | a carrier (solvent) for the active ingredients above. |

### Production Example 7: Topical Finasteride and Minoxidil

| Ingredient | Function |
|---|---|
| Finasteride (0.1-2.5%) | DHT blocker; inhibits 5α-reductase locally |
| Minoxidil (3-5%) | Vasodilator; stimulates hair regrowth |
| Ethanol, Propylene glycol | Solvents; enhance penetration and stability |
| Purified water | Solvent/diluent |

### Production Example 8: Topical Finasteride and Minoxidil

| Ingredient | Function |
|---|---|
| Finasteride (2.5%) | DHT blocker; inhibits 5α-reductase locally |
| Minoxidil (3%) | Vasodilator; stimulates hair regrowth |
| SiloxysSystem ™ Gel | Silicone-based gel; slow-release, enhances scalp penetration |
| Ethanol, Propylene glycol | Solvents; enhance penetration and stability |
| Purified water | Solvent/diluent |

### Production Example 9: Topical Minoxidil

| Ingredient | Function |
|---|---|
| Minoxidil (5%) | Vasodilator; stimulates hair regrowth |
| Ethanol, Propylene glycol | Solvents; enhance penetration and stability |
| Purified water | Solvent/diluent |

### Production Example 10: Tropical Minoxidil

| Ingredient | Function |
|---|---|
| Minoxidil (2% or 5%) | Vasodilator; stimulates hair regrowth |
| Ethanol, Propylene glycol | Solvents; enhance penetration and stability |
| Purified water | Solvent/diluent |

### Production Example 11: Tropical Saw Palmetto, Biotin, and Marine Collagen

| Ingredient | Function |
|---|---|
| Saw palmetto | Inhibits 5α-reductase; reduces DHT; supports follicle health |
| Biotin | Supports keratin production; improves hair strength |
| Marine collagen | Provides amino acids for hair structure; supports follicle health |
| Ashwagandha | Adaptogen; reduces stress-related hair loss |
| Tocotrienols | Antioxidant; protects follicles from oxidative stress |
| Zinc | Cofactor for hair growth enzymes; regulates DHT and immune |
| | function |
| Vitamin C | Antioxidant; supports collagen synthesis and scalp health |
| Flax seed | Source of omega-3 fatty acids; anti-inflammatory; supports |
| | scalp health |
| Other | Additional support for hair and scalp health |
| botanicals/micronutrients | |

### Production Example 12: Topical Marine Collagen

| Ingredient | Function |
|---|---|
| Marine collagen | Provides amino acids for hair structure; supports follicle health |
| Vitamin C | Antioxidant; supports collagen synthesis and scalp health |
| Zinc | Cofactor for hair growth enzymes; regulates DHT and immune |
| | function |
| Flax seed | Source of omega-3 fatty acids; anti-inflammatory; supports |
| | scalp health |

### Production Example 13: Topical SANDALORE™

| Ingredient | Function |
|---|---|
| SANDALORE ™ (3-methyl- | Olfactory receptor agonist; prolongs anagen phase; reduces |
| 5-(2,2,3-trimethylcyclopent- | hair shedding |
| 3-en-1-yl)pentan-2-ol) 1% | |
| Caffeine | Stimulates follicle proliferation; counteracts DHT; improves |
| | microcirculation |
| Lauric acid | Antimicrobial; reduces scalp inflammation; supports follicle |
| | health |
| Niacinamide (Vitamin B3) | Improves scalp circulation; strengthens hair shaft; anti- |
| | inflammatory |

### Production Example 14: Topical Minoxidil

| Ingredient | Function |
|---|---|
| Minoxidil (5% w/v) | Active. Potassium channel opener; stimulates hair follicles to |
| | promote regrowth |
| Ethyl Alcohol | Solvent. Enhances solubility and penetration; helps preserve |
| | formulation |
| Propylene Glycol | Humectant/solvent. Retains moisture; aids in dissolving |
| | minoxidil |
| Purified Water | Solvent/vehicle. Adjusts viscosity and volume |

### Production Example 15: Topical Minoxidil 5% Foam (Alcohol/Propylene Glycol-Free)

| Ingredient | Function |
|---|---|
| Minoxidil (5.0 wt. %) | Active ingredient. Stimulates hair follicles; promotes regrowth |
| Polysorbate 20 (1.0%) | Surfactant; stabilizes foam |
| Cetyl alcohol (3.0%) | Emollient; supports foam structure; moisturizes skin |
| Stearyl alcohol (2.5%) | Emollient; supports foam structure; moisturizes skin |
| Glycerin (5.0%) | Humectant; retains moisture; enhances skin compatibility |
| Purified water (qs) | Vehicle for dissolution; adjusts viscosity/volume |
| Fragrance (0.1%) | Sensory modifier; improves user experience |
| Preservative (e.g., | Antimicrobial; ensures product stability |
| phenoxyethanol, 0.5%) | |

### Production Example 16: Topical Minoxidil 5%+Finasteride 0.25% Foam

| Ingredient | Function |
|---|---|
| Minoxidil (5.0 wt. %) | Active ingredient. Stimulates hair follicles; promotes regrowth |
| Finasteride (0.25 wt. %) | Active ingredient. DHT blocker; inhibits 5α-reductase locally |
| Polysorbate 20 (1.0%) | Surfactant; stabilizes foam |
| Cetyl alcohol (3.0%) | Emollient; supports foam structure; moisturizes skin |
| Stearyl alcohol (2.5%) | Emollient; supports foam structure; moisturizes skin |
| Glycerin (5.0%) | Humectant; retains moisture; enhances skin compatibility |
| Purified water (qs) | Vehicle for dissolution; adjusts viscosity/volume |
| Fragrance (0.1%) | Sensory modifier; improves user experience |
| Preservative (e.g., | Antimicrobial; ensures product stability |
| phenoxyethanol, 0.5%) | |

### Production Example 17: Topical Minoxidil 5%+Ketoconazole 1% Solution

| Ingredient | Function |
|---|---|
| Minoxidil (5.0 wt. %) | Active ingredient. Stimulates hair follicles; promotes regrowth |
| Ketoconazole (1.0%) | Active ingredient. Antifungal; reduces DHT; controls scalp |
| | microbiome |
| Ethyl Alcohol (30%) | Solvent; enhances penetration and product stability |
| Propylene Glycol (10%) | Solvent; humectant; improves API delivery |
| Purified water (qs) | Solvent/vehicle; adjusts viscosity and volume |
| Preservative (0.5%) | Ensures formulation stability |

### Production Example 18: Topical Minoxidil 5%+Saw Palmetto 3% Solution

| Ingredient | Function |
|---|---|
| Minoxidil (5.0 wt. %) | Active ingredient. Stimulates hair follicles; promotes regrowth |
| Saw Palmetto extract (3%) | Botanical DHT blocker; supports follicle health |
| Ethyl Alcohol (30%) | Solvent; enhances penetration and product stability |
| Propylene Glycol (10%) | Solvent; humectant; improves API delivery |
| Purified water (qs) | Vehicle; adjusts viscosity and volume |
| Preservative (0.5%) | Ensures formulation stability |

### Production Example 19: Topical Multi-Peptide Serum for Hair Density (Botanical-Free, Cosmetic Adjuvant)

| Ingredient | Function |
|---|---|
| Acetyl tetrapeptide-3 | Peptide. Stimulates follicle matrix |
| (0.01%) | proteins; density |
| Biotinoyl tripeptide-1 | Peptide. Enhances follicle anchoring |
| (0.02%) | |
| Myristoyl pentapeptide-17 | Peptide. Prolongs anagen phase |
| (0.005%) | |
| Panthenol (1.0%) | Provitamin B5; strengthens and moisturizes |
| | hair |
| Hyaluronic acid (0.5%) | Humectant; improves scalp hydration |
| Glycerin (5.0%) | Humectant; retains moisture |
| Propanediol (2.0%) | Solvent; skin friendly |
| Phenoxyethanol (0.5%) | Preservative |
| Purified water (qs) | Solvent; adjusts viscosity and volume |

### Production Example 20: Topical Rosemary Oil Serum

| Ingredient | Function |
|---|---|
| Rosemary oil (2.6%) | Botanical active; stimulates hair follicles; |
| | anti-inflammatory |
| Jojoba oil (5.0%) | Skin conditioning agent; supports hair health |
| Squalane (2.0%) | Conditioning agent; occlusive |
| Vitamin E (0.2%) | Antioxidant; stabilizes product |
| Polysorbate 20 (1.0%) | Surfactant; solubilizes rosemary oil |
| Phenoxyethanol (0.5%) | Preservative |
| Purified water (qs) | Vehicle/diluent |

### Production Example 21: Topical Redensyl Peptide Serum

| Ingredient | Function |
|---|---|
| Redensyl (5%) | Synthetic stem cell stimulator; activates ORSc |
| | stem cells and prolongs the anagen phase |
| Aqua (Purified Water) | Solvent/vehicle |
| Glycerin (3%) | Humectant; retains moisture |
| Dihydroquercetin- | Antioxidant component of Redensyl; promotes |
| glucoside (1%) | hair follicle cell activity |
| EGCG-glucoside (1%) | Antioxidant; supports follicle health |
| Glycine (2%) | Amino acid; provides nutrients to follicles |
| Zinc chloride (0.5%) | Follicle nutrition and health |
| Sodium benzoate (0.5%) | Preservative; ensures formulation stability |
| Phenoxyethanol (0.5%) | Preservative |
| Parfum (0.1%) | Sensory modifier; improves user experience |

### Production Example 22. Caffeine Shampoo

| Ingredient | Function |
|---|---|
| Aqua (Purified Water) | Solvent/vehicle |
| Sodium Laureth Sulfate | Surfactant; cleansing agent |
| (10%) | |
| Cocamidopropyl Betaine | Surfactant; improves foam and mildness |
| (5%) | |
| Caffeine (1-2%) | Active ingredient; stimulates hair |
| | follicle metabolism; supports growth |
| Panthenol (1%) | Provitamin B5; strengthens and |
| | moisturizes hair |
| PEG-7 Glyceryl Cocoate | Emollient; improves conditioning |
| (1%) | |
| Citric Acid (0.5%) | pH adjuster; maintains product stability |
| Sodium benzoate (0.5%) | Preservative; ensures product stability |
| Polyquaternium-10 (0.2%) | Film former; imparts hair smoothness |

### Production Example 23: Pure Rosemary Oil Topical Solution

| Ingredient | Function |
|---|---|
| Rosemary oil (2.6%) | Botanical active; stimulates hair follicles; |
| | anti-inflammatory |
| Jojoba oil (5%) | Conditioning agent; supports scalp health |
| Squalane (2%) | Conditioning agent; occlusive; supports |
| | barrier function |
| Polysorbate 20 (1%) | Emulsifier; solubilizes rosemary oil |
| Phenoxyethanol (0.5%) | Preservative |
| Aqua (Purified Water) (qs) | Vehicle/diluent |

### Production Example 24: Capixyl/Baicapil Peptide Complex Serum

| Ingredient | Function |
|---|---|
| Capixyl (Butylene Glycol, | Peptide/botanical complex; stimulates |
| Acetyl Tetrapeptide-3, | ECM proteins and prolongs anagen phase |
| Trifolium pratense extract; | |
| 5%) | |
| Baicapil (5%) | Botanical/peptide complex; stimulates hair |
| | growth, delays catagen phase |
| Glycerin (3%) | Humectant; retains moisture |
| Aqua (Purified Water) | Solvent/vehicle |
| Betaine (1%) | Osmoprotectant; moisturizes scalp |
| Phenoxyethanol (0.5%) | Preservative |
| Sodium benzoate (0.5%) | Preservative |

### Production Example 25: Topical Growth Factor & EGF Serum

| Ingredient | Function |
|---|---|
| Recombinant human EGF | Growth factor; stimulates follicle cell |
| (0.05%) | proliferation |
| bFGF (0.05%) | Growth factor; promotes hair follicle |
| | regeneration |
| KGF (0.01%) | Growth factor; enhances follicle vitality |
| Hyaluronic acid (0.5%) | Humectant; scalp hydration |
| Glycerin (3%) | Humectant; retains moisture |
| Aqua (Purified Water) (qs) | Solvent/vehicle |
| Sodium benzoate (0.5%) | Preservative |

### Production Example 26: Trioxidil-Based Serum

| Ingredient | Function |
|---|---|
| Trioxidil proprietary | Multi-ingredient complex; supports follicle |
| blend (5%) | health and microcirculation |
| Minoxidil (2%) [if | Potassium channel opener; stimulates hair |
| applicable] | regrowth |
| Biotin (0.05%) | Supports keratin production; strengthens hair |
| Aqua (Purified Water) | Solvent/vehicle |
| Glycerin (3%) | Humectant; retains moisture |
| Phenoxyethanol (0.5%) | Preservative |
| Sodium benzoate (0.5%) | Preservative |

### Production Example 27: Topical Caffeine-Only Solution

| Ingredient | Function |
|---|---|
| Caffeine (3%) | Active ingredient; stimulates follicle |
| | metabolism and local microcirculation |
| Ethanol (20%) | Solvent; enhances penetration |
| Propylene glycol (10%) | Solvent/humectant; supports API delivery |
| Aqua (Purified Water) (qs) | Vehicle/diluent |
| Panthenol (0.5%) | Pro-vitamin B5; supports hair shaft |
| | hydration |
| Sodium benzoate (0.2%) | Preservative |

### Production Example 28: Topical Peptide+Minoxidil+Redensyl Serum

| Ingredient | Function |
|---|---|
| Minoxidil (5%) | Vasodilator; stimulates hair regrowth |
| Redensyl (3%) | Stem cell stimulator; activates follicle |
| | progenitor cells |
| Biotinoyl tripeptide-1 | Peptide; enhances follicle anchoring |
| (0.01%) | |
| Myristoyl pentapeptide-17 | Peptide; prolongs the anagen phase |
| (0.005%) | |
| Glycerin (3%) | Humectant; retains moisture |
| Aqua (Purified Water) (qs) | Vehicle/diluent |
| Phenoxyethanol (0.5%) | Preservative |

### Production Example 29: Leave-In Conditioner with DHT Blockers

| Ingredient | Function |
|---|---|
| Saw palmetto | Botanical DHT blocker; supports follicle |
| extract (3%) | health |
| Niacinamide (2%) | Vitamin B3; improves scalp circulation; |
| | barrier function |
| Panthenol (1%) | Pro-vitamin B5; strengthens and moisturizes |
| | hair |
| Caffeine (1%) | Stimulates follicle metabolism |
| Polyquaternium- | Film former; leaves-in hair conditioning |
| 10 (0.2%) | |
| Aqua (Purified | Vehicle/diluent |
| Water) (qs) | |
| Phenoxyethanol | Preservative |
| (0.5%) | |

### Production Example 30: Shampoo with Redensyl and Biotin

| Ingredient | Function |
|---|---|
| Sodium Laureth | Primary surfactant; cleanses hair |
| Sulfate (10%) | |
| Cocoamidopropyl | Secondary surfactant; improves foam and |
| betaine (5%) | mildness |
| Redensyl (1%) | Synthetic stem cell stimulator for hair |
| | regrowth |
| Biotin (0.1%) | Supports keratin production; strengthens |
| | hair |
| Panthenol (1%) | Pro-vitamin B5; moisturizes and strengthens |
| | hair |
| Glycerin (2%) | Humectant; retains moisture |
| Citric acid (0.3%) | pH adjuster; maintains stability |
| Polyquaternium-10 (0.2%) | Film former; adds smoothness |
| Sodium benzoate (0.5%) | Preservative; ensures formulation stability |
| Aqua (Purified Water) (qs) | Solvent/vehicle |

### Example 1: Topical Formulation

Any one or more of the above Production Examples 1-10 and 14 can be modified to replace at least a portion of the solvent (ethyl alcohol, alcohol, water, propylene glycol) with any one or more of. (1) penetration enhancer, (2) co-solvent, (3) additional API, (4) vitamin/nutraceutical natural product, (5) antioxidant/chelator, (6) pH adjuster/buffer, (7) film-former & polymer, (8) conditioning agent, (9) CMC (cell membrane complex) mimic, (10) anti-inflammatory agent, (11) antimicrobial/preservative, (12) humectant, (13) sensory modifier, (14) penetration modulator, (15) stabilizer, (16) color stabilizer, (17) chelating agent, and (18) emulsifier/surfactant, added to any one or more of the above Production Examples 1-10 and 14 is shown.

Likewise, any one or more of the above Production Examples 11-13 can be modified to add any one or more of. (1) penetration enhancer, (2) co-solvent, (3) additional API, (4) vitamin/nutraceutical natural product, (5) antioxidant/chelator, (6) pH adjuster/buffer, (7) film-former & polymer, (8) conditioning agent, (9) CMC (cell membrane complex) mimic, (10) anti-inflammatory agent, (11) antimicrobial/preservative, (12) humectant, (13) sensory modifier, (14) penetration modulator, (15) stabilizer, (16) color stabilizer, (17) chelating agent, and (18) emulsifier/surfactant, added to any one or more of the above Production Examples 11-13 is shown.

Example 1: Topical Formulations

| | Unique | |
|---|---|---|
| | Ingredient | Amount/ |
| | Identifier | Range |
| Additional Substance(s) | (UNII) | (% w/w) |
| 1. Penetration enhancer: | | |
| Transcutol ® (diethylene | A1A1I8X02B | ≤10% |
| glycol monoethyl ether) | | |
| Cetyl alcohol | 936JST6JCN | 1-10% |
| Oleic acid | 2UMI9U37CP | ≥6% |
| Eucalyptol | RV6J6604TK | 1-5% |
| Polyethylene glycol | Q662QK8M3B | 5-15% |
| Octyl salicylate | 4W5IH7FLNY | 1-5% |
| SiloxysSystem ™ | UNII | 1-8% |
| Gel (manufacturer | 5V2I96E4WJ | |
| proprietary blend) | | |
| Laurocapram | 1F3X9DRV9X | 0.1-3% |
| Dimethyl Isosorbide (DMI) | SA6A6V432S | 5-15% |
| Urea (Carbamide) | 8W8T17847W | 5-10% |
| d-Limonene | 9MC3I34447 | 1-5% |
| Menthol | L7T10EIP3A | 0.1-1.2% |
| α-Bisabolol | 36HQN158VC | 0.1-2% |
| Azone ® (Laurocapram) | 1F3X9DRV9X | 0.1-3% |
| 2. Co-solvent: | | |
| Rosemary oil | 8LGU7VM393 | ≤2.6% |
| Peppermint oil | AV092KU4JH | 1-3% |
| Lavender oil | ZBP1YXW0H8 | 3-5% |
| Thyme oil | 2UK410MY6B | 1-2% |
| Cedarwood oil | PAD4FN7P2G | 1-3% |
| | (Eastern | |
| | red cedar), | |
| | ZX5QRE4U60 | |
| | (Atlas cedar) | |
| Lemongrass oil | XE7K568ILO | 5-15% |
| | (general) | |
| Lemongrass oil | 5BIA40E9ED | 5-15% |
| | (West Indian) | |
| Clary sage oil | 87L0D4U3M0 | 1-3% |
| Tea tree oil | VIF565UC2G | 1-5% |
| Ylang-ylang oil | 8YOY78GNNX | 0.5-2% |
| Jojoba oil | 724GKU717M | 5-10% |
| Pumpkin seed oil | 6E5QR5USSP | 2-5% |
| Cannabidiol (CBD) oil | 19GBJ60SN5 | 0.1-1% |
| Clove oil | 8U1V72KE9E | 1-5% |
| Soya lecithin | 1DI6B4I9SH | 1-5% |
| Ethoxydiglycol | A1A1I8X02B | 1-10% |
| Isopropyl alcohol | ND2M416302 | 10-30% |
| 3. Additional API: | | |
| Clobetasol propionate | 0X9XU3S2H9 | 0.05% |
| Betamethasone | LMR652FZVK | 0.05-0.1% |
| Fluocinolone acetonide | 0CD5FD6S2M | 0.01-0.025% |
| Fluocinonide | 2W4A77YPAN | 0.05% |
| Anthralin | U8CJK0JH5M | 0.1-1% |
| Diphenylcyclopropenone | I7G14NW5EC | 0.001-2% |
| (DPCP) | | |
| Squaric acid dibutylester | 4RTO57VG65 | 0.001-2% |
| (SADBE) | | |
| Clascoterone | 5V2I96E4WJ | 0.5-5% |
| Pyrilutamide (KX-826) | XN7MM8XG2M | 0.1-0.5% |
| ET-02 (Eirion Therapeutics) | N/A | 1-5% |
| Breezula ® (Clascoterone 5%) | 5V2I96E4WJ | 1-5% |
| PP405 (Pelage Pharmaceuticals) | N/A | 1-5% |
| Cyclosporine | 83HN0GTJ6D | 0.05-0.1% |
| Ketoconazole | R9400W927I | 1-2% |
| Bimatoprost | O5C5V00OXU | 0.005-0.03% |
| SCD-153 (4-MI prodrug) | N/A | 1-5% |
| Capsaicin | S07O44R1ZM | 0.025-0.1% |
| Isoflavones | N/A | 1-7.5% |
| Cetirizine | XUZ4Z382L0 | 0.1-1% |
| Flutamide | G7G0G0Y5P9 | 0.1-1% |
| 4. Vitamin / nutraceutical / | | |
| natural product: | | |
| Caffeine | 3G6A5W338E | 0.1-3% |
| Saw palmetto | J7WWH9M8QS | 1-5% |
| Biotin | 6SO6U10H04 | 0.001-0.1% |
| Niacin (Vitamin B3) | 2679MF687A | 2-5% |
| Green tea extract | 2JQ7048N98 | 2-5% |
| Procyanidin B-2 | F7V2S3P5R8 | 1-2% |
| Capsaicin | S07O44R1ZM | 0.025-0.1% |
| Melatonin | JL5DK93RCL | 0.01-0.1% |
| Zinc | J41CSQ7QDS | 0.1-2% |
| Marine collagen | 5LFD6G6X6H | 1-10% |
| Hyaluronic acid | 7QXD1P34BO | 0.1-2% |
| Tocotrienols (Vitamin E) | R0ZB2556P8 | 0.5-2% |
| Glycyrrhizin (licorice) | 6FO62043WK | 0.1-1% |
| Aloe Vera | ZY81Z83H1X | 0.5-2% |
| 5. Antioxidant/Chelator: | | |
| Butylated Hydroxytoluene | VU05PDE4YW | 0.01-0.1% |
| (BHT) | | |
| EDTA (Disodium EDTA) | 7FLD91C86K | 0.01-0.5% |
| Ascorbic Acid | PQ6CK8PD0R | 0.1-10% |
| (Vitamin C) | | |
| Alpha-Tocopherol | R0ZB2556P8 | 0.1-1% |
| (Vitamin E) | | |
| Tocotrienols | O03Y90O6QN | 0.1-1% |
| (Vitamin E family) | | |
| Rosemary CO₂ Extract | 8LGU7VM393 | ≤2.6% |
| Sodium Phytate | 8T9K2Z539X | 0.01-0.5% |
| (Phytic Acid salt) | | |
| Citric Acid | 2968PHW8QP | 0.05-1% |
| Sodium Gluconate | R6Q379206O | 0.1-1% |
| Beta-Carotene | 01YAE03M7J | 0.01-0.1% |
| Lycopene | 01M8A3O4M9 | 0.01-0.05% |
| Caffeine | 3G6A5W338E | 0.1-3% |
| Green Tea Extract (EGCG) | 2JQ7048N98 | 2-5% |
| Niacinamide (Vitamin B3) | 25X5118RD4 | 1-5% |
| Glycyrrhizin (Licorice) | 6FO62043WK | 0.1-1% |
| 6. pH Adjuster/Buffer: | | |
| Citric Acid | 2968PHW8QP | 0.05-2% |
| (anhydrous/monohydrate) | | |
| Lactic Acid | 33X04XA5AT | 0.1-2% |
| Acetic Acid (glacial) | Q40Q9N063P | 0.01-1% |
| Phosphoric Acid | 1VQO26JZZ5 | 0.01-1% |
| Hydrochloric Acid | QTT17582CB | q.s. (as needed) |
| Tartaric Acid | W4888I119H | 0.01-1% |
| Sodium Hydroxide | 55X04QC32I | q.s. (as needed) |
| Potassium Hydroxide | 019I9Y1549 | q.s. (as needed) |
| Sodium Bicarbonate | 8MDF5V39QO | 0.1-1% |
| Sodium Carbonate | 451W47IQ8X | 0.1-1% |
| Sodium Phosphate Monobasic | 04QHX5986D | 0.1-2% |
| Sodium Phosphate Dibasic | 87R18W38CQ | 0.1-2% |
| Tris (TRIS, Tromethamine) | 023C2WHX2V | 0.1-1% |
| HEPES | J3J8Q68W8K | 0.1-1% |
| BIS-TRIS | 4K8W6N8A7W | 0.1-1% |
| Glycylglycine | 8U2X8U5I6G | 0.1-1% |
| Ammonium Hydroxide | NH2PNP02JR | q.s. (as needed) |
| Monoethanolamine | 5KV86114PT | q.s. (as needed) |
| Diethanolamine | AZI9I9U3LH | q.s. (as needed) |
| Tromethamine (TRIS) | 023C2WHX2V | 0.1-1% |
| Sodium Borate | 91MBZ8H3QO | 0.1-1% |
| 7. Film-former & Polymer: | | |
| Polyquaternium-10 | Q81F4E4N0D | 0.2-2% |
| PVP (Polyvinylpyrrolidone) | KX7Q0DTJ1C | 0.5-5% |
| Zinc Pyrithione | R596H2102T | ≤2% |
| Hydroxyethylcellulose | X6Q6TXF6UM | 0.1-1% |
| 8. Conditioning Agent: | | |
| Ceramide NP | 67HFT40120 | 0.01-0.2% |
| Panthenol | 82RDE2NM6R | 0.1-5% |
| Hydrolyzed keratin | 6U5W9VH7GF | 0.01-5% |
| 9. CMC (Cell Membrane | | |
| Complex) Mimic: | | |
| Ceramide | 67HFT40120 | 0.01-0.2% |
| Cholesterol | 97C5T2UQ7J | 0.005-3% |
| Fatty acids (e.g., | 9KJL21T0QJ | 0.1-5% |
| Linoleic acid) | | |
| 10. Anti-inflammatory | | |
| Agent: | | |
| Allantoin ® | 344S277G0Z | 0.1-2% |
| Bisabolol | 36HQN158VC | 0.1-1% |
| Glycyrrhetinic acid | 6FO62043WK | 0.01-2% |
| 11. Antimicrobial/ | | |
| Preservative: | | |
| Phenoxyethanol | I6W7S6Y37P | 0.5-1% |
| Sodium benzoate | OJ245FE5EU | 0.2-1% |
| Benzyl alcohol | LKG8494WBH | 0.5-1.0% |
| Caprylyl glycol | 8WQ5659F5T | 0.5-1.0% |
| Potassium sorbate | 1VPU26JZZ4 | 0.025-0.5% |
| 12. Humectant: | | |
| Glycerin | PDC6A3C0OX | 2-10% |
| Sodium PCA | K72T3FS567 | 0.2-2.5% |
| Urea | 8W8T17847W | 0.5-10% |
| 13. Sensory Modifier: | | |
| Menthol | L7T10EIP3A | 0.1-1.2% |
| Fragrance blends | N/A | 0.01-1% |
| (manufacturer proprietary) | | |
| 14. Penetration Modulator: | | |
| Cyclodextrins | J6K4F9V3BA | 0.1-2% |
| Liposomes | N/A | 0.5-5% |
| (manufacturer proprietary) | | |
| 15. Stabilizer: | | |
| Trehalose | B8WCK70T7I | 0.5-5% |
| 16. Color Stabilizer: | | |
| Sodium metabisulfite | 4VON4I66K8 | 0.01-0.1% |
| Ascorbyl palmitate | 8CG0I00Z2E | 0.01-0.5% |
| 17. Chelating Agent: | | |
| EDTA | 7FLD91C86K | 0.01-0.5% |
| Sodium phytate | 8T9K2Z539X | 0.01-0.5% |
| 18. Emulsifier/Surfactant: | | |
| Polysorbate 20 | 7T1F30V5YH | 0.1-2% |
| Cetearyl alcohol | 329ZB9U5LL | 1-5% |
| Polysorbate 80 | 6OZP39ZG8H | 0.1-2% |
| Carbomer | 8LVP2ISJ0T | 0.1-1% |

### Example 2(a): Administration Protocol

1. Apply 1 mL formulation (topical composition of Example 1) to clean scalp, 1-2 times daily.

2. Repeat daily ×6 months.

### Example 2(b): Administration Protocol, Microneedling

Use 0.3 mm roller with 192 needles (5 passes) prior to step 1 of Example 2(a).

### Example 2(c): Administration Protocol, Microneedling

Use 0.3 mm roller with 192 needles (5 passes) after step 1 of Example 2(a).

### Example 2(d): Administration Protocol, Low-level Light Therapy

Use low-level light therapy (LLLT) device in combination with the administration protocol of any one of Examples 1-2(c). Key technical specifications of the device include:

Wavelength: 650-830 nm red/infrared spectrum, with 650 nm most clinically proven for follicle stimulation.

Energy Density: ≥3 J/cm² per session.

Light Source: True laser diodes (not LEDs) for coherent, deeper scalp penetration.

Pulse Wave Emission: Rapid on/off cycling enhances efficacy vs. continuous waves.

Coverage: ≥300 diodes arranged for full scalp targeting.

Treatment Frequency: 10-30 minutes daily/every other day, balancing efficacy and compliance.

### Example 2(e): Administration Protocol, Oral Administration of Active Ingredient(s)

The administration protocol of any one of Examples 1-2(d) is carried out, with the co-administration of any one or more of:

Finasteride: 1 mg orally once daily for male pattern hair loss.

Minoxidil: 1.25-5 mg orally once daily for men (common starting dose: 1.25 mg; up to 5 mg based on response and tolerance). For women, lower doses (0.5-1.25 mg daily).

Dutasteride: 0.5 mg orally once daily.

### Example 3(a): Analytical Methods for Measuring Specification Parameters of Hair Regrowth Formulations

| Measurable Parameter | Analytical Method (Standard Protocol) |
|---|---|
| Particle Size (D10/D50/D90) | USP <429> (laser diffraction) or USP <430> (dynamic light |
| | scattering): Measure using Malvern Mastersizer 3000 (wet |
| | dispersion) or Shimadzu SALD-2300 (undiluted emulsions |
| | per Application Note 01-00231-EN) |
| Span Value | Calculate via (D90 − D10)/D50 using USP <429> data. |
| | Validate with ISO 13320 for dry/wet comparison |
| Viscosity | USP <911> (Brookfield RV DV-III Ultra, spindle #7, 20 rpm) |
| | or VROC technology (ASTM D2196 for thixotropic index) |
| Drying Time | GB/T 1728-79 (filter paper method: 10 μL/cm² application) |
| | or ASTM D5909-20 (squalene resistance test for oxidative- |
| | drying systems) |
| Chemical Stability | ICH Q1A(R2): Accelerated testing (40° C./75% RH × 6 |
| | months), quantify API degradation via HPLC |
| Transepidermal Water Loss | ISO 24444: Measure with Vapometer ® (forearm baseline vs. |
| | 30-min post-application) |
| pH | USP <791>: Calibrate with pH 4.0/7.0 buffers, measure using |
| | Mettler Toledo SevenExcellence ™ probe. |
| Anagen/Telogen Ratio | Trichoscopy (70x videodermoscopy): Classify ≥100 follicles |
| | via root morphology (Saitoh's method) |
| Content Uniformity | USP <905>: Test 10 units via HPLC (RSD ≤6% for APIs) |
| Yield Stress | ASTM D6572: Controlled-stress rheometer (TA Instruments |
| | AR-G2, 25° C., 40 mm plate) |
| Thixotropic Index | ASTM D2196: Calculate as viscosity at 10 rpm/viscosity at |
| | 100 rpm (Brookfield RV DV-III Ultra) |
| Penetration Enhancer | Franz cell assay (OECD 428): Compare follicular deposition |
| Efficiency | ratios (test vs. control) at 8 h using HPLC |
| Antioxidant Efficacy | ICH Q1A(R2): Accelerated stability testing (40° C./75% RH) |
| | with HPLC quantification of API degradation over 6 months |
| Undiluted PSD | SALD-2300 Laser Diffraction: Measure emulsions without |
| | dilution per Shimadzu Application Note 01-00231-EN. |
| Evaporation Rate | TGA/DSC (ISO 11358-1): Measure weight loss at 32° C. over |
| | 240 min |
| Osmolality | USP <785>: Measure via freezing-point depression |
| | (Advanced Instruments 3250 Single-Sample Osmometer). |
| Particulate Matter | USP <788>: Light obscuration particle count for particles ≥10 |
| | μm and ≥25 μm |
| Epidermal Retention Time | Tape stripping + HPLC/LC-MS |
| User Treatment Compliance | Digital logs/app monitoring |
| Content Uniformity | Dose validation, HPLC |
| (Application) | |
| Residence Time (Matrix | Imaging/drug tracer study |
| Systems) | |
| Photostability | ICH Q1B, HPLC |
| Companion Device/Sensor | Device readout, ISO-compliant protocols |
| Outputs | |
| Scalp Microbiome Diversity | 16S rRNA sequencing, diversity index |
| Inflammatory Biomarker | Tape-strip ELISA |
| Levels | |
| Cost-Efficiency/Application | Dose volume, gravimetric analysis |
| Number | |
| Content Uniformity (Dose) | HPLC of dispensed volumes |
| Scalp Microbiome Diversity | 16S rRNA sequencing, diversity index |

### Example 3(b): Analytical Methods for Measuring Clinical Endpoints/Outcomes

| Measurable Parameter | Clinical Outcome Protocol |
|---|---|
| Hair Density | ISO 29800: 2023 (phototrichogram) or TrichoScan ® HD (70x |
| | magnification, ≥100 hairs/cm²) |
| Terminal Hair Count (THC) | Trichoscopy (70x videodermoscopy): Count ≥100 terminal |
| | hairs (≥30 μm diameter) per 4 cm² |
| Vellus Hair Count (VHC) | Trichoscopy (70x videodermoscopy): Count ≥100 vellus hairs |
| | (<30 μm diameter) per 4 cm² |
| Follicular Deposition | OECD 428 Franz cell assay: Porcine ear skin, HPLC analysis |
| | at 4 h/8 h/24 h. Compare test vs. control ratios |
| DHT Reduction | ARG80838 ELISA Kit: Serum analysis via competitive |
| | binding (450 nm absorbance, 60-min protocol) |
| Anagen Phase Duration | Trichogram with root microscopy: Classify ≥50 plucked hairs |
| | via Saitoh's method (J Dermatol Sci. 2007) |
| Hair Thickness/Diameter | ISO 29800: 2023 via TrichoScan ® HD (70x magnification, ≥100 |
| | hairs analyzed) |
| Scalp Keratin Levels | Bradford assay (ISO 20776-1): Quantify soluble keratin from |
| | scalp tape strips using Bio-Rad Protein Assay |
| Hair Length/Growth Rate | Phototrichogram (ISO 29800: 2023): Track elongation of |
| | marked hairs over 14 days using CASLite Nova |
| Terminal-to-Vellus Hair Ratio | Calculate as THC/VHC using ISO 29800: 2023 trichoscopy |
| (T/V Ratio) | data |
| Hair Regrowth/New Hair | Phototrichogram (ISO 29800: 2023): Track new hair |
| Formation | emergence in bald areas over 12 weeks |
| Hair Tensile Strength | Testronix tensile hair tester (TX TST C): Measure force-to- |
| | break (N) at 5 mm/min extension rate |
| Norwood-Hamilton/Ludwig | Clinical assessment by board-certified dermatologist per AAD |
| Classification | guidelines |
| Scalp Coverage | Imaging analysis, planimetry |
| Immediate Cosmetic Coverag | Photography, panel ratings, image analysis |
| User Treatment Compliance | Digital logs/app monitoring |
| Patient-Reported Outcomes | DLQI, Hairdex questionnaires |
| (PROs) | |
| Hair Shine/Luster | Glossmeter, panel scoring |
| Scalp Sensitivity/Pruritus | VAS, patch testing, questionnaires |
| Regrowth Pattern Mapping | Trichoscopy mapping/image stitching |
| Immediate Cosmetic | Image analysis, panel scoring |
| Coverage | |
| Scalp Microbiome Diversity | 16S rRNA sequencing, diversity index |
| Trichorrhexis/Tinea Capitis | Microscopy, fungal culture |
| Dx | |

All analytical instruments in Examples 3(a) and 3(b) should be calibrated using standard reference particles or chemical standards prior to each series of analyses. Method validation must include linearity, accuracy, precision, and specificity for all APIs and excipients present, particularly in multi-API compositions with potential chromatographic interference.

1. A topical composition comprising:
(a) active pharmaceutical ingredient comprising at least one of finasteride, minoxidil, dutasteride, and latanoprost;
(b) retinoic acid;
(c) at least one of hydrocortisone and aloe vera, in amounts effective to reduce irritation;
(d) solvent comprising at least one of propylene glycol, ethanol, and water;
(e) 0.1-5 wt. % bioadhesive polymer, to prolong scalp residence time and maintain hair follicle contact;
(f) penetration enhancer, for follicular targeting, comprising at least one of Transcutol®, cetyl alcohol, oleic acid, eucalyptol, polyethylene glycol, octyl salicylate, SiloxysSystem™ gel, laurocapram, dimethyl isosorbide, urea, d-limonene, menthol, and α-bisabolol, Azone®;
(g) at least one of an antioxidant, chelator, pH adjuster, film-former/polymer, humectant, sensory modifier, penetration modulator, stabilizer, chelating agent, emulsifier/surfactant, vitamin/nutraceutical natural product, and antimicrobial/anti-inflammatory agent.

2. The topical composition of claim 1, wherein the composition provides at least 40% greater follicular API deposition and at least 30% less irritation compared to a composition otherwise identical but lacking the penetration enhancer, as measured by standard ex vivo follicular diffusion and irritation assays.

3. The topical composition of claim 1, wherein the penetration enhancer increases follicular API deposition by ≥40% compared to a formulation without the penetration enhancer, measured by Franz cell assay.

4. The topical composition of claim 1, further comprising at least one of:
additional API,
vitamin/nutraceutical natural product,
antioxidant/chelator,
pH adjuster,
film-former or polymer,
humectant,
sensory modifier,
penetration modulator,
stabilizer,
chelating agent, and
emulsifier/surfactant,
each present in an amount and combination sufficient to enhance scalp tolerability, bioadhesion, release control, antimicrobial activity, and active stability.

5. The topical composition of claim 1,
formulated as a bioadhesive hydrogel, smart-responsive polymer matrix, slow-release gel, nanoparticle/liposome suspension, or patch; or
delivered via microneedle array for localized follicular deposition;
wherein the active pharmaceutical ingredient is present as particles having D90 ≤8.5 μm and a Span value ≤1.7.

6. The topical composition of claim 1, wherein the composition is provided in a kit comprising:
(i) a multi-chambered, modular, or sequential dispensing dispenser or applicator, that permits customizable mixing and/or sequencing of actives, penetration enhancer, co-solvents, or adjunctives at the point of use;
(ii) at least one of scalp diagnostic feedback system, calibrated dropper, wearable device, companion app, and user-tailored modular components for regimen personalization;
(iii) at least one of a calibrated dropper delivering 1.0 mL±0.05 mL per actuation, an amber pharmaceutical-grade glass bottle, and a closure with a UV-protective coating configured to reduce photodegradation of actives by ≥40% over six months.

7. The topical composition of claim 1, wherein the penetration enhancer increases follicular deposition of the active ingredient(s) by ≥40% compared to formulations without the penetration enhancer, as measured by in vitro Franz cell diffusion assay using porcine ear skin.

8. The topical composition of claim 1, wherein the penetration enhancer reduces epidermal retention time of the active ingredient(s) to ≤15 minutes while maintaining ≥60% follicular penetration efficiency, and/or achieve ≥30% reduction in transepidermal water loss (TEWL) within 30 minutes post-application.

9. The topical composition of claim 1, that retains at least ≥70% potency after 12 months at 25° C./60% RH, with chemical/integrity stability confirmed by ICH Q1A(R2) protocols.

10. The topical composition of claim 1, further comprising exosomes or stem cell-conditioned media (1-5% w/w) supporting follicle regeneration, wherein such regenerative ingredients are blended with actives and penetration enhancer for synergistic effect.

11. The topical composition of claim 1, further comprising an antioxidant system comprising at least one of BHT, EDTA, tocotrienols, and ascorbic acid, present at a level sufficient to reduce or prevent ≥98% active pharmaceutical ingredient degradation on accelerated stability testing.

12. The topical composition of claim 1, further comprising a pH-modifying buffer system capable of dynamically adjusting formulation to pH 5.7-6.4 at the time of administration.

13. The topical composition of claim 1, further comprising an antimicrobial or anti-inflammatory agent at least one of zinc pyrithione, ketoconazole, and bisabolol, present in amounts effective for reducing scalp irritation, supporting microbiome balance, and/or providing antifungal protection.

14. A method of treating alopecia, hair loss, or promoting hair growth, comprising topically administering the composition to the scalp of a human in need thereof, wherein topical administration is performed with or without device-assisted delivery, such as microneedling, microcurrent activation, ultrasound, or LLLT;
the topical composition comprising:
(a) active pharmaceutical ingredient comprising at least one of finasteride, minoxidil, dutasteride, and latanoprost;
(b) retinoic acid;
(c) at least one of hydrocortisone and aloe vera, in amounts effective to reduce irritation;
(d) solvent comprising at least one of propylene glycol, ethanol, and water;
(e) 0.1-5 wt. % bioadhesive polymer, to prolong scalp residence time and maintain hair follicle contact;
(f) penetration enhancer, for follicular targeting, comprising at least one of Transcutol®, cetyl alcohol, oleic acid, eucalyptol, polyethylene glycol, octyl salicylate, SiloxysSystem™ gel, laurocapram, dimethyl isosorbide, urea, d-limonene, menthol, and α-bisabolol, Azone®;
(g) at least one of an antioxidant, chelator, pH adjuster, film-former/polymer, humectant, sensory modifier, penetration modulator, stabilizer, chelating agent, emulsifier/surfactant, vitamin/nutraceutical natural product, and antimicrobial/anti-inflammatory agent.

15. The method of claim 14, wherein the subject is characterized by androgenetic alopecia, alopecia areata, or female pattern hair loss, and the regimen may be adjusted based on scalp feedback, sensor data, or digital guidance.

16. The method of claim 14, further comprising at least one of:
microneedling (0.1-1.5 mm),
low-level light therapy (650-830 nm),
sensor-guided dosing,
app-based regimen scheduling,
co-administration of oral 5α-reductase inhibitor or topical antifungal agent, and
the use of a self-heating or phase-change carrier system to enhance API penetration above scalp temperature (≥32° C.).

17. The method of claim 14, achieving ≥25% greater follicular deposition and ≥30% reduction in local irritation relative to ethanol-based active pharmaceutical ingredient formulas, as measured by ISO or clinical protocols, with content uniformity and real-time device validation as per USP/ISO standards.

18. The method of claim 14, retaining at least 90% API potency after 12 months at 25° C./60% RH, determined by ICH Q1A(R2) chemical stability testing.

19. The method of claim 14, producing a ≥20% increase in scalp hair density at 24 weeks versus untreated controls, measured by trichoscopy.

20. The method of claim 14, that stops or slows hair loss, and promotes growth by blocking dihydrotestosterone (DHT).

21. The method of claim 14, that regrows hair and prolongs the growth (anagen) phase of hair follicles.

22. The method of claim 14, that promotes denser hair regrowth.

23. The method of claim 14, that inhibits both type-1 and type-2 5-alpha reductase, which otherwise converts testosterone to DHT.

24. The method of claim 14, that increases hair density and stimulates healthy hair follicle activity.

25. The method of claim 14, wherein regimen adjustment is guided by scalp sensor feedback, user digital input, or machine learning, optimizing dose, ingredient selection, or formulation based on real-time trichoscopy or scalp condition data.

26. The method of claim 14, wherein topical administration is performed with simultaneous or sequential device-assisted delivery, such as ultrasound or microcurrent activation, enhancing API penetration by at least 25% compared to passive application.

27. The method of claim 14, wherein the regimen comprises stepwise follicle priming with caffeine, retinoic acid, or glycolic acid, followed by main API administration for improved efficacy.

28. A kit comprising:
(i) a pharmaceutical-grade glass bottle,
(ii) a closure with a UV-protective coating,
(iii) a calibrated dropper system delivering 1.0 mL±0.05 mL per actuation,
(iv) optionally a companion at least one of an app, diagnostic sensor, and multi-chambered modular delivery system;
wherein the pharmaceutical-grade glass bottle contains a topical composition comprising:
(a) active pharmaceutical ingredient comprising at least one of finasteride, minoxidil, dutasteride, and latanoprost;
(b) retinoic acid;
(c) at least one of hydrocortisone and aloe vera, in amounts effective to reduce irritation;
(d) solvent comprising at least one of propylene glycol, ethanol, and water;
(e) 0.1-5 wt. % bioadhesive polymer, to prolong scalp residence time and maintain hair follicle contact;
(f) penetration enhancer, for follicular targeting, comprising at least one of Transcutol®, cetyl alcohol, oleic acid, eucalyptol, polyethylene glycol, octyl salicylate, SiloxysSystem™ gel, laurocapram, dimethyl isosorbide, urea, d-limonene, menthol, and α-bisabolol, Azone®;
(g) at least one of an antioxidant, chelator, pH adjuster, film-former/polymer, humectant, sensory modifier, penetration modulator, stabilizer, chelating agent, emulsifier/surfactant, vitamin/nutraceutical natural product, and antimicrobial/anti-inflammatory agent.

29. The kit of claim 28, wherein the closure reduces API photodegradation by ≥40% over 6 months.

30. The kit of claim 28, wherein the pharmaceutical-grade glass bottle comprises pharmaceutical grade amber glass.
